(19) United States
(12) Patent Application Publication
(10) Pub. No.: US 2010/0216762 A1
(43) Pub. Date: Aug. 26, 2010

(54) Agonists and Antagonists of the S1P5 Receptor, and Methods of Use Thereof

(75) Inventors: Christopher M. Harris, Shrewsbury, MA (US); Adrian D. Hobson, Shrewsbury, MA (US); Noel S. Wilson, Kenosha, WI (US)

Correspondence Address: ABBOTT BIORESEARCH, 100 RESEARCH DRIVE, WORCESTER, MA, 01605-4314, US

(73) Assignee: ABBOTT LABORATORIES, Abbott Park, IL (US)

(21) Appl. No.: 12/703,615

(22) Filed: Feb. 10, 2010

Related U.S. Application Data
(60) Provisional application No. 61/207,301, filed on Feb. 10, 2009.

(51) Int. Cl. A61K 31/397; C07D 205/04; C07D 209/82; C07D 401/02; A61P 25/00; A61P 25/30; A61P 25/28; A61P 29/00

(52) U.S. Cl. 514/210.17; 548/953; 548/444; 546/268.1; 514/210.18

(57) ABSTRACT

Disclosed are compounds that are agonists or antagonists of the S1P₅ receptor, compositions comprising said compounds, and methods of using said compounds and compositions. In certain embodiments, said compounds are 1-benzylazetidine-3-carboxylic acid derivatives. In certain embodiments, said methods relate to the treatment of neuropatic pain and/or a neurodegenerative disorder. In certain embodiments, said compounds may be used in combination with a second therapeutic agent.

### RELATED APPLICATION

This application claims priority to and the benefit of U.S. Provisional Application No. 61/207,301, filed Feb. 10, 2009.

### BACKGROUND

Sphingosine-1-phosphate (S1P) is part of the sphingomyelin biosynthetic pathway and is known to affect multiple biological processes. S1P is formed through phosphorylation of sphingosine by sphingosine kinases (SK1 and SK2), and it is degraded through cleavage by sphingosine lyase to form palmitaldehyde and phosphoethanolamine or through dephosphorylation by phospholipid phosphatases. S1P is present at high levels (about 500 nM) in serum, and it is found in most tissues. S1P can be synthesized in a wide variety of cells in response to several stimuli, which include cytokines, growth factors and G protein-coupled receptor (GPCR) ligands. The GPCRs that bind S1P (currently known as the S1P receptors S1P₁₋₅), couple through pertusis toxin sensitive (Gi) pathways as well as pertusis toxin insensitive pathways to stimulate a variety of processes. The individual receptors of the S1P family are both tissue and response specific and, therefore, are attractive as therapeutic targets.

S1P evokes many responses from cells and tissues. In particular, S1P has been shown to be an agonist at all five GPCRs, S1P₁ (Edg-1), S1P₂ (Edg-5), S1P₃ (Edg-3), S1P₄ (Edg-6) and S1P₅ (Edg-8). The action of S1P at the S1P receptors has been linked to resistance to apoptosis, changes in cellular morphology, cell migration, growth, differentiation, cell division, angiogenesis, oligodendrocyte differentiation and survival, modulation of axon potentials, and modulation of the immune system via alterations of lymphocyte trafficking. Therefore, S1P receptors are therapeutic targets for the treatment of, for example, neoplastic diseases, diseases of the central and peripheral nervous system, autoimmune disorders and tissue rejection in transplantation. These receptors also share 50-55% amino acid identity with three other lysophospholipid receptors, LPA1, LPA2, and LPA3, of the structurally related lysophosphatidic acid (LPA).

GPCRs are excellent drug targets with numerous examples of marketed drugs across multiple disease areas. GPCRs are cell-surface receptors that bind hormones on the extracellular surface of the cell and transduce a signal across the cellular membrane to the inside of the cell. The internal signal is amplified through interaction with G proteins, which in turn interact with various second messenger pathways. This transduction pathway is manifested in downstream cellular responses that include cytoskeletal changes, cell motility, proliferation, apoptosis, secretion and regulation of protein expression, to name a few. S1P receptors make good drug targets because individual receptors are expressed in different tissues and signal through different pathways, making the individual receptors both tissue and response specific. Tissue specificity of the S1P receptors is desirable because development of an agonist or antagonist selective for one receptor localizes the cellular response to tissues containing that receptor, limiting unwanted side effects. Response specificity of the S1P receptors is also of importance because it allows for the development of agonists or antagonists that initiate or suppress certain cellular responses without affecting other responses. For example, the response specificity of the S1P receptors could allow for an S1P mimetic that initiates platelet aggregation without affecting cell morphology.

The physiologic implications of stimulating individual S1P receptors are largely unknown due in part to a lack of receptor type selective ligands. Isolation and characterization of S1P analogs that have potent agonist or antagonist activity for S1P receptors have been limited.

S1P₁ for example is widely expressed, and the knockout causes embryonic lethality due to large vessel rupture. Adoptive cell transfer experiments using lymphocytes from S1P₁ knockout mice have shown that S1P₁ deficient lymphocytes sequester to secondary lymph organs. Conversely, T cells overexpressing S1P₁ partition preferentially into the blood compartment rather than secondary lymph organs. These experiments provide evidence that S1P₁ is the main sphingosine receptor involved in lymphocyte homing and trafficking to secondary lymphoid compartments.

The S1P₂ receptor is expressed in many types of smooth muscle tissue, as well as on mast cells, macrophages, dendritic cells, eosinophils, and endothelial cells. S1P stimulation of S1P₂ induces airway smooth muscle contractility and potentiates the airway response to cholinergic stimulation (Kume, H., et al. (2007) J Pharm Exp Ther 320, 766-773). A requirement for S1P₂ signaling has been shown for IgE receptor-mediated mast cell degranulation (Jolly, P. S. et al. (2004) J. Exp. Med. 199, 959-970). The S1P-induced increase in paracellular permeability of endothelial cell cultures is S1P₂-dependent, and S1P₂ antagonism blocks vascular permeability in hydrogen peroxide-challenged perfused lung (Sanchez, T., et al. (2007) Arterioscler Thromb Vasc Biol 27, 1312-1318). Antagonism of the S1P₂ receptor also induced hypertension (US 2006/0148844 A1). In addition, S1P₂ has been shown to be involved in the pathologic neovascularization following ischemia-driven retinopathy (Skoura, A. et al. (2007) J. Clin. Invest. 117, 2506-2516).

The S1P₃ receptor is expressed in heart, lung, kidney, and the immune system. S1P₃ stimulation induces contraction of smooth muscle in many tissues, potentially overlapping with S1P₂ function. In addition, S1P₃ modulates airway resistance, barrier integrity, and the bradycardia associated with non-selective S1P receptor agonists. (Sanna, M. G., et al. (2004) J. Biol. Chem. 279, 13839-13848; Gon, Y (2005) Proc Natl. Acad. Sci., 102, 9270-9275).

Of the S1P receptor family, S1P₄ has the most restrictive expression profile, being expressed solely in the immune system. Highest expression of S1P₄ is on neutrophils, NK cells, B cells, T cells, and monocytes. The physiological function of S1P₄ is poorly understood.

The S1P₅ receptor is expressed in the central nervous system, predominantly on oligodendrocytes and neurons, and in the immune system, mainly on natural killer cells, T cells, and neutrophils. S1P₅ regulates the migration of oligodendrocyte precursor cells and transiently induces their process retraction. In addition, S1P₅ stimulation promotes survival of mature olidodendrocytes (Jaillard, C., et al. (2005) J. Neuroscience 25, 1459-1469, Novgorodov, A. S., et al. (2007) FASEB J 21, 1503-1541). In vivo migration of natural killer cells under homeostatic or inflammatory conditions requires the S1P₅ receptor (Walzer, T., et al. (2007) Nature Immunology 8, 1337-1344).

Sphingolipids are essential plasma-membrane lipids that are concentrated in lipid rafts or cholesterol-enriched membrane microdomains. These lipids can be rapidly metabolized following stimulation of various plasma-membrane receptors through the activation of an enzymatic cascade that converts sphingolipids, such as sphingomyelin or complex glycosphingolipids, to ceramide and subsequently to sphingosine. Two sphingosine kinases (SK1 and SK2) then phosphorylate sphingosine to sphingosine-1-phosphate (S1P). Ceramide and S1P regulate opposing biological processes; ceramide results in oxidative stress, is pro-apoptotic, and induces cell death, whereas as S1P stimulates cell survival and proliferation (Rivera, et al. (2008) Nat. Rev. Immun. 8, 753-763; Cuvillier, O. et al. (1996) Nature 381, 800-803).

Altered sphingolipid metabolism is strongly implicated in neurodegenerative and cognitive diseases. A comparison of gene expression profiles in normal and Alzheimer's Disease (AD) brains indicated that genes responsible for S1P degradation were strongly upregulated, including the phosphatidic acid phosphatase PPAP2A and S1P lyase genes, while genes for S1P production were unchanged. Also, the majority of genes involved in de novo ceramide synthesis were upregulated and their expression levels correlated with disease severity (Katsel, P. et al. (2007) Neurochem. Res. 32, 845-856). Gene expression data are predictive of actual changes in enzyme and lipid levels. Compared to normal, AD brains are characterized by higher levels of ceramide, sphingosine, and cholesterol as well as decreases in sphingomyelin and S1P. Changes in lipid levels also correlate with disease severity for these patients (Cutler, R. G. et al. (2004) Proc. Nat. Acad. Sci. 101, 2070-2075; He, X. et al. (2010) Neurobiol. Aging 31, 398-408). The same changes in sphingolipids and cholesterol have been reported in brain tissue from patients suffering from HIV dementia and amyotrophic lateral sclerosis, suggesting high ceramide and low S1P may be hallmarks of neurodenerative diseases (Cutler, R. G. et al (2002) Ann. Neurol. 52, 448-457; Haughey, N. J. et al. (2004) Ann. Neurol. 55, 257-267; Cutler, R. G. et al. (2010) Neurology 63, 626-630). Modulating the activity of one or more S1P receptors in the central nervous system may be a therapeutic method for neurodegenerative or cognitive diseases by shifting the ceramide/S1P balance toward S1P effects and away from ceramide-mediated cell death.

Soluble β-amyloid (Aβ) oligomers are considered the proximate effectors of the synaptic injury and neuronal death occurring in the early stages of AD. Aβ induces increased ceramide levels and oxidative stress in neuronal cultures, leading to apoptosis and cell death. S1P is a potent neuroprotective factor against this Aβ-induced damage, consistent with its role in opposing the effects of ceramide ((Cutler, R. G. et al. (2004) Proc. Nat. Acad. Sci. 101, 2070-2075; Malaplate-Armand, C. et al. (2006) Neurobiol. Dis. 23, 178-189). Aβ is also pro-inflammatory, inducing the migration of monocytes to sites of injury, and the S1P₁, S1P₃, S1P₄, S1P₅ receptor agonist FTY720 inhibits that migration. Aβ induces the expression of the S1P receptors S1P₂ and S1P₅, but not S1P₁, S1P₃, or S1P₄, (Kaneider, N. C. et al. (2004) FASEB J 10.1096/fj.03-1050fje). The profiles of S1P receptors acted upon by FTY720 and those expressed by monocytes suggests these effects are mediated by the S1P₅ receptor.

Additional studies suggest a role for S1P in modulating pain signals. S1P modulates action potentials in capsaicin-sensitive sensory neurons (Zhang, Y. H. et al. (2006) J Physiol. 575, 101-113; Zhang, Y. H. et al. (2006) J. Neurophyiol. 96, 1042-1052). S1P levels are decreased in the cerebral spinal fluid in acute and inflammatory pain models, and reducing S1P levels through deletion of the SK1 gene exacerbated paw withdrawal latency in the formalin model. Intrathecal S1P inhibits cAMP, a key second messenger of spinal nociceptive processing, and abolishes cAMP-dependent phosphorylation of NMDA receptors in the spinal cord, a mechanism of central sensitization to pain (Coste, O. et al. (2008) J. Biol. Chem. 283, 32442-32451). The S1P₁, S1P₃, S1P₄, S1P₅ receptor agonist FTY720 is anti-nociceptive in the formalin model under conditions that do not cause S1P₁-mediated immunosuppression, and FTY720 reduced nociceptive behavior in the spared-nerve injury model of neuropathic pain (Coste, O. et al. (2008) J. Cell. Mol. Med. 12, 995-1004). The lack of activity for the S1P₁-selective agonist SEW2871 in the formalin model and the high CNS expression of S1P₅ suggest this receptor as the one that mediates S1P effects in pain.

Potent and selective agents that are agonists or antagonists of the individual receptors of the S1P receptor family are needed to address unmet medical needs associated with agonism or antagonism of the individual receptors of the S1P receptor family. More specifically, agonists or antagonists of S1P₅ will be beneficial for the treatment of cognitive disorders, neurodegenerative diseases, and pain. In particular, S1P₅-selective ligands would be beneficial for these diseases by not causing the immune suppression resulting from S1P₁ modulation.

### SUMMARY

The invention relates to in part to compounds that are agonists or antagonists of the individual receptors of the S1P receptor family, compositions comprising such compounds, and methods of using such compounds and compositions.

Another aspect of the invention relates to a pharmaceutical composition comprising a compound of the invention, or pharmaceutically acceptable salt or prodrug thereof, and one or more pharmaceutically acceptable carriers, alone or in combination with another therapeutic agent. Such pharmaceutical compositions of the invention can be administered in accordance with a method of the invention, typically as part of a therapeutic regimen for treatment or prevention of conditions and disorders related to individual receptors of the S1P receptor family.

Another aspect of the invention relates to a method of treating a neurodegenerative disorder or neuropathic pain comprising administering to a subject in need thereof a therapeutically effective amount of one or more compounds or pharmaceutical compositions of the invention. In certain embodiments, said neurodegenerative disorder is selected from the group consisting of neurodegenerative diseases selected from Alzheimer's disease, Huntington's disease, Parkinson's disease, Amyotrophic Lateral Sclerosis, asphyxia, acute thromboembolic stroke, focal and global ischemia and transient cerebral ischemic attacks. In certain embodiments, the compound or pharmaceutical composition comprises a compound of formula II, or a pharmaceutically acceptable salt, biologically active metabolite, solvate, hydrate, prodrug, enantiomer or stereoisomer thereof.

### DETAILED DESCRIPTION

One aspect the invention provides a method for agonizing or antagonizing S1P₅ in a human subject suffering from a disorder in which modulation of S1P₅ activity is beneficial, comprising administering to the human subject a compound of the invention such that S1P₅ activity in the human subject is altered and treatment is achieved.

For example, a compound of the invention, or a pharmaceutically acceptable salt, biologically active metabolite, solvate, hydrate, prodrug, enantiomer or stereoisomer thereof, or pharmaceutical compositions containing a therapeutically effective amount thereof, is useful in the treatment of a disorder selected from the group comprising Alzheimer's disease, arthritis, rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, infertility, female infertility, ovarian failure, premature ovarian failure, fibrotic lung disease, chronic wound healing, cryptogenic fibrosing alveolitis, post-inflammatory interstitial lung disease, fibrosis, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multisystem disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occlusive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynaud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute pain, age-associated memory impairment (AAMI), anxiety attention deficit disorder, attention deficit disorder in general, attention deficit hyperactivity disorder (ADHD), bipolar disorder, cancer pain, central neuropathic pain syndromes, central post-stroke pain, chemotherapy-induced neuropathy, cognitive deficits and dysfunction in psychiatric disorders, cognitive deficits associated with aging and neurodegeneration, cognitive deficits associated with diabetes, cognitive deficits of schizophrenia, complex regional pain syndrome, declines in cognitive function in Alzheimer's and associated dementias, deficits in attention, dementia, dementia associated with Down's syndrome, dementia associated with Lewy bodies, depression in Cushing's syndrome, diminished CNS function associated with traumatic brain injury, diseases with deficits of memory, dizziness, drug abuse, epilepsy, HIV sensory neuropathy, Huntington's disease, hyperalgesia including neuropathic pain, inflammation and inflammatory disorders, inflammatory hyperalgesia, inflammatory pain, insulin resistance syndrome, jet lag, lack of circulation, learning, major depressive disorder, medullary thyroid carcinoma, Meniere's disease, metabolic syndrome, mild cognitive impairment, mood alteration, motion sickness, multiple sclerosis pain, narcolepsy, need for new blood vessel growth associated with vascularization of skin grafts and lack of circulation, need for new blood vessel growth associated with wound healing, neuropathic pain, neuropathy, neuropathy secondary to tumor infiltration, non-inflammatory pain, obesity, obsessive compulsive disorder, painful diabetic neuropathy, panic disorder, Parkinson disease pain, pathological sleepiness, phantom limb pain, Pick's Disease, polycystic ovary syndrome, post traumatic stress disorder, post-herpetic neuralgia, post-mastectomy pain, post-surgical pain, psychotic depression, schizoaffective disorder, seizures, senile dementia, sepsis syndrome, sleep disorders, smoking cessation, spinal cord injury pain, steroid-induced acute psychosis, sub-categories of neuropathic pain including peripheral neuropathic pain syndromes, substance abuse including alcohol abuse, Syndrome X, Tourette's syndrome, treatment resistant depression, trigeminal neuralgia, type II diabetes, vertigo, and vestibular disorders.

As noted above, one aspect of the invention relates to the use of a compound of the invention in the treatment of neuropathic pain. Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain, pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has disS1Pated, commonly lasting for years, significantly decreasing a patient's quality of life. The symptoms of neuropathic pain are difficult to treat, as they are often heterogeneous even between patients with the same disease. They include spontaneous pain, which can be continuous, and paroxysmal or abnormal evoked pain, such as hyperalgesia (increased sensitivity to a noxious stimulus) and allodynia (sensitivity to a normally innocuous stimulus).

### SUMMARY OF THE INVENTION

One embodiment of the invention relates to a compound represented by Formula (I)

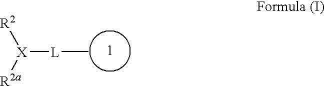

or a pharmaceutically acceptable salt, biologically active metabolite, solvate, hydrate, prodrug, enantiomer or stereoisomer thereof; wherein,
Ring 1 is optionally substituted aryl or optionally substituted heteroaryl;
L is —N(R^{a})—, —O— or C(R^{a})₂; wherein
   R^{a} is independently H or optionally substituted alkyl;
X is N when L is C(R^{a})₂ or
X is CR^{a}; when L is —N— or —O—;
R² and R^{2a} are independently hydrogen, optionally substituted alkyl, optionally substituted alkenyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹; wherein
   W is O or S; and
   R¹¹ is —OR, —N(R)₂ or —SR; wherein
      R is independently hydrogen, optionally substituted alkyl or haloalkyl; or
when X is N or C, R² and R^{2a} together with the carbon or nitrogen atom to which they are attached form an optionally substituted cycloalkyl, optionally substituted azetidine, optionally substituted pyrrolidine, optionally substituted piperidine or optionally substituted octahydrocyclopenta[c]pyrrolyl ring, provided that the azetidine ring formed by R² and R^{2a} together with the carbon or nitrogen atom to which they are attached is not substituted by
   one or more phenyl;
   phenyl and OH;
   phenyl and —N(H)C(CH₃)₃;
   —CH₂—O-optionally substituted pyridinyl;
   —NH-optionally substituted quinazolinyl;
   —O-optionally substituted pyridinyl;
   —O—Si(CH₃)₂—C(CH₃)₃;
   —C(OH)(4-(trifluoromethoxy)phenyl)(4-methoxyphenyl);
   —C(OH)(4-(trifluoromethoxy)phenyl)(4-methoxyphenyl) and oxo;
   —NH-isoquinolinyl;
   optionally substituted alkyl and optionally substituted dioxolanyl;
   oxo and —O-alkenyl;
   oxo, two F and optionally substituted phenyl;
   optionally substituted alkenyl and —O—C(O)-optionally substituted phenyl;
provided that when Ring 1 is optionally substituted phenyl, L is CH₂ X is N or C, and R² and R^{2a} together with the carbon or nitrogen atom to which they are attached form an optionally substituted cycloalkyl, or optionally substituted azetidine, Ring 1 is not substituted by
   —CH═N—OCH₂CH₃;
   —Cl and —NH₂;
   —C(═O)CH₂CH₂-optionally substituted oxazolyl;
   —NH—C(O)-alkenyl-optionally substituted pyridinyl;
   —NO₂ and COOH—O-alkyl-optionally substituted oxazolyl;
   —O—CH₂-optionally substituted benzofuranyl;
   —O—CH₂-optionally substituted phenyl;
   —O—CH₂-optionally substituted pyrazolyl;
   —O—CH₂-optionally substituted thienyl;
   —O-optionally substituted (C₈)alkyl;
   —O-optionally substituted (C₈)alkyl and halo;
   —(C₆-C₁₂)alkyl wherein one or more carbons is optionally replaced by a nonperoxide oxygen;
   —(C₆-C₁₂)alkenyl wherein one or more carbons is optionally replaced by a nonperoxide oxygen;
   -pyrimidinyl substituted with oxo and —CF₂CF₃;
   -optionally substituted 1,2,4 oxadiazole;
   -optionally substituted thiazolo[5,4-b]pyridine;
   -optionally substituted phenyl-CH₂—C(O)-optionally substituted pyrazolyl;
   -optionally substituted phenyl-CH₂—C(O)-optionally substituted thiazolyl;
   -optionally substituted phenyl-NH—C(O)-optionally substituted pyrazolyl;
   -optionally substituted phenyl-NH—C(O)-optionally substituted tetrazolyl;
   -optionally substituted phenyl-NH—C(O)-optionally substituted triazolyl;
   -optionally substituted pyridinyl-CH₂—C(O)-optionally substituted pyrazolyl;
   -optionally substituted pyridinyl-CH₂—C(O)-optionally substituted thiazolyl;
   -optionally substituted pyridinyl-NH—C(O)-optionally substituted pyrazolyl;
   -optionally substituted pyridinyl-NH—C(O)-optionally substituted tetrazoyl;
   -optionally substituted pyridinyl-NH—C(O)-optionally substituted triazolyl;
   -optionally substituted pyrimidinyl-CH₂—C(O)-optionally substituted pyrazolyl;
   -optionally substituted pyrimidinyl-NH—C(O)-optionally substituted pyrazolyl;
   -optionally substituted pyrimidinyl-NH—C(O)-optionally substituted triazolyl;
   -optionally substituted phenyl-CH₂—C(O)-optionally substituted triazolyl;
provided that when Ring 1 is optionally substituted isoxazolyl or optionally substituted oxazolyl, Ring 1 is not substituted by
   -optionally substituted phenyl-optionally substituted bicycle[2.2.1]heptanyl;
   -optionally substituted phenyl-optionally substituted alkyl-optionally substituted phenyl;
provided that when Ring 1 is optionally substituted pyridinyl, Ring 1 is not substituted by
   —C(O)—NH-optionally substituted phenyl;
   —O-optionally substituted phenyl; and
provided that when Ring 1 is optionally substituted phenyl or naphthyl, L is CH₂ and NR² and NR^{2a} form an optionally substituted pyrrolidine ring, the pyrrolidine ring is not substituted by
   —C(═O)(OH);
   —F and —C(═O)(OH);
   —OH and —C(═O)(OH);
   —P(═O)(OH)(OH);
   —OH and —P(═O)(OH)(OH);
   —CH₂C(═O)(OH); or
   tetrazolyl.

Another embodiment of the invention relates to a compound according to any of the foregoing embodiments wherein Ring 1 is optionally substituted benzofuranyl, optionally substituted benzimidazolyl, optionally substituted dibenzofuranyl, optionally substituted benzothiazolyl, optionally substituted benzothienyl, 9H-carbazolyl, optionally substituted cinnolinyl, optionally substituted fluorenyl, optionally substituted furanyl, optionally substituted imidazolyl, optionally substituted indazolyl, optionally substituted indenyl, optionally substituted indolizinyl, optionally substituted indolyl, optionally substituted isoindolyl, optionally substituted 3H-indolyl, optionally substituted isothiazolyl, optionally substituted isoxazolyl, optionally substituted naphthyridinyl, optionally substituted naphthalenyl, optionally substituted oxadiazolyl, optionally substituted oxazolyl, optionally substituted phthalazinyl, optionally substituted pteridinyl, optionally substituted purinyl, optionally substituted phenyl, optionally substituted pyrazolyl, optionally substituted pyridazinyl, optionally substituted pyridinyl, optionally substituted pyrimidinyl, optionally substituted pyrrolyl, optionally substituted quinazolinyl, optionally substituted quinoxalinyl, optionally substituted quinolizinyl, optionally substituted quinolinyl, optionally substituted isoquinolinyl, optionally substituted tetrazolyl, optionally substituted thienyl, or optionally substituted triazolyl.

Another embodiment of the invention relates to according to any of the foregoing embodiments wherein -L-X(R²)(R^{2a}) form

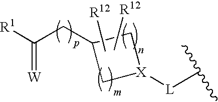

wherein
R¹ is hydrogen, hydroxy, optionally substituted alkyl, optionally substituted alkoxy, haloalkoxy or haloalkyl, —(CH₂), —O—P(═O)(OR⁷)(OR⁷), —(CH₂)ₓ—P(═O)(OR⁷)(OR⁷), —(CH₂)ₓ—P(═O)(OR⁷)(R⁷), —CH═CH—P—(═O)(OR⁷)(OR⁷);
   wherein R⁷ is hydrogen, optionally substituted alkyl or optionally substituted phenyl; and
   x is 0 or 1;
R^{a} is hydrogen, optionally substituted alkyl or haloalkyl;
R¹² is independently hydrogen, hydroxy, optionally substituted alkyl, halo, or —(CH₂)ₚC(═W)R¹¹;
m is 1, 2 or 3;
n is 0, 1 or 2 and
p is 0 or 1.

Another embodiment of the invention relates to a compound according to any of the foregoing embodiments wherein the compound is
1-((1-(phenylsulfonyl)-1H-indol-3-yl)methyl)azetidine-3-carboxylic acid;
1-(1-(9H-carbazol-2-yl)ethyl)azetidine-3-carboxylic acid;
1-(dibenzo[b,d]furan-3-ylmethyl)azetidine-3-carboxylic acid;
1-((5-(phenylethynyl)thiophen-2-yl)methyl)azetidine-3-carboxylic acid;
1-((2-(4-methoxybenzoyl)benzofuran-5-yl)methyl)azetidine-3-carboxylic acid;
1-((5-(4-bromophenyl)isoxazol-3-yl)methyl)azetidine-3-carboxylic acid;
1-((6-(3,4-dichlorophenyl)pyridin-3-yl)methyl)azetidine-3-carboxylic acid;
1-((6-(4-(trifluoromethyl)phenyl)pyridin-3-yl)methyl)azetidine-3-carboxylic acid;
1-((6-(benzyloxy)pyridin-3-yl)methyl)azetidine-3-carboxylic acid;
1-((6-(3,4-dichlorobenzyloxy)pyridin-3-yl)methyl)azetidine-3-carboxylic acid;
1-((5-(4-methoxyphenyl)thiophen-2-yl)methyl)azetidine-3-carboxylic acid;
1-((5-(4-chlorophenyl)thiophen-2-yl)methyl)azetidine-3-carboxylic acid;
1-((5-(4-fluorophenyl)thiophen-2-yl)methyl)azetidine-3-carboxylic acid;
1-((5-(4-(trifluoromethyl)phenyl)thiophen-2-yl)methyl)azetidine-3-carboxylic acid;

1-((5-(4-fluorophenyl)thiophen-2-yl)methyl)azetidine-3-carboxylic acid;
1-((5-o-tolylthiophen-2-yl)methyl)azetidine-3-carboxylic acid;
1-((5-m-tolylthiophen-2-yl)methyl)azetidine-3-carboxylic acid;
1-((5-p-tolylthiophen-2-yl)methyl)azetidine-3-carboxylic acid;
1-((5-(3-(trifluoromethyl)phenyl)thiophen-2-yl)methyl)azetidine-3-carboxylic acid;
1-((5-(3,4-dimethoxyphenyl)thiophen-2-yl)methyl)azetidine-3-carboxylic acid;
1-((5-phenylthiophen-2-yl)methyl)azetidine-3-carboxylic acid;
1-((3′,4′-dichlorobiphenyl-4-yl)methyl)azetidine-3-carboxylic acid;
1-((4′-ethylbiphenyl-4-yl)methyl)azetidine-3-carboxylic acid;
1-((2′-methoxybiphenyl-4-yl)methyl)azetidine-3-carboxylic acid;
1-((2′-chlorobiphenyl-4-yl)methyl)azetidine-3-carboxylic acid; or
1-((2′-methylbiphenyl-4-yl)methyl)azetidine-3-carboxylic acid.

Another embodiment of the invention relates to a compound according to any of the foregoing embodiments wherein the compound is

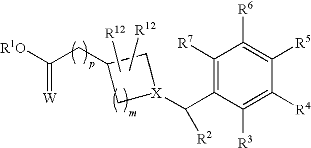

wherein
R³, R⁴, R⁶, and R⁷ are independently selected from the group consisting of optionally substituted alkenyl, optionally substituted alkoxy, optionally substituted alkoxycarbonyl, optionally substituted alkoxysulfonyl, optionally substituted alkyl, optionally substituted alkylcarbonyl, optionally substituted alkylcarbonyloxy, optionally substituted alkylsulfonyl, optionally substituted alkylthio, optionally substituted alkynyl, optionally substituted aryl, optionally substituted aryloxy, amido, optionally substituted amino, carboxy, cyano, formyl, halo, haloalkoxy, haloalkyl, hydrogen, hydroxyl, hydroxyalkyl, mercapto, nitro, silyl and silyloxy;

R⁵ is optionally substituted aryl, optionally substituted arylalkyl, optionally substituted arylalkylcarbonyl, optionally substituted 2-thiazolyl, optionally substituted arylalkoxy, optionally substituted arylalkylthio, optionally substituted arylcarbonyloxy, optionally substituted arylcarbonylalkoxy, optionally substituted aryloxycarbonyl, optionally substituted arylalkenyl, optionally substituted alkyl, optionally substituted alkylcarbonyl, optionally substituted alkenyl, optionally substituted alkynyl, optionally substituted alkenyloxy, optionally substituted aryloxy, optionally substituted alkoxy, optionally substituted alkoxycarbonyl, haloalkoxy, optionally substituted cycloalkoxy, optionally substituted alkenyloxy, optionally substituted arylalkynyl, optionally substituted benzo[d][1,3]dioxolyl, optionally substituted cycloalkyl, optionally substituted cycloalkyloxy, optionally substituted heterarylalkyl, optionally substituted heteroaryl or optionally substituted heteroarylalkyloxy.

Another embodiment of the invention relates to a compound according to any of the foregoing embodiments wherein R⁵ is halogen, optionally substituted alkyl, optionally substituted alkynyl, optionally substituted alkoxy, optionally substituted alkenyloxy, optionally substituted alkyloxycarbonyl, optionally substituted benzo[d][1,3]dioxolyl, optionally substituted benzyl, optionally substituted benzylcarbonyl, optionally substituted benzylthio, optionally substituted benzyloxy, optionally substituted cycloalkyloxy, optionally substituted naphthyl, optionally substituted aryl, optionally substituted arylalkenyl, optionally substituted arylcarbonyloxy, optionally substituted arylalkyl, optionally substituted aryloxy, optionally substituted pyridinyl, optionally substituted thiazolyl, optionally substituted thienyl, or optionally substituted thienylalkoxy.

Another embodiment of the invention relates to a compound according to any of the foregoing embodiments wherein R⁵ is halogen, optionally substituted alkyl, optionally substituted alkynyl, optionally substituted alkoxy, optionally substituted alkenyloxy, optionally substituted alkyloxycarbonyl, optionally substituted benzo[d][1,3]dioxolyl, optionally substituted benzyl, optionally substituted benzylcarbonyl, optionally substituted benzylthio, optionally substituted benzyloxy, optionally substituted cycloalkyloxy, optionally substituted naphthyl, optionally substituted phenyl, optionally substituted phenylalkenyl, optionally substituted phenylcarbonyloxy, optionally substituted phenylethyl, optionally substituted phenyoxy, optionally substituted pyridinyl, optionally substituted thiazolyl, optionally substituted thienyl, or optionally substituted thienylalkoxy.

Another embodiment of the invention relates to a compound according to any of the foregoing embodiments wherein R⁵ is optionally substituted by one or more substituents independently selected from the group consisting of —C(O)-optionally substituted alkyl, —C(O)-optionally substituted alkoxy, —C(O)-optionally substituted phenyl, —O-optionally substituted cycloalkyl, optionally substituted alkoxy, optionally substituted alkyl, halo, CF₃, cyano, nitro, oxo, optionally substituted phenyl, or trimethylsilylalkynyl.

Another embodiment of the invention relates to a compound according to any of the foregoing embodiments wherein X is N.

Another embodiment of the invention relates to a compound according to any of the foregoing embodiments wherein the compound is
1-((4′-methylbiphenyl-4-yl)methyl)azetidine-3-carboxylic acid;
1-(4-(2-chlorobenzyloxy)-3-methoxybenzyl)azetidine-3-carboxylic acid;
1-(4-(2-chlorobenzyloxy)-3-ethoxybenzyl)azetidine-3-carboxylic acid;
1-(4-(4-chlorobenzyloxy)-3-ethoxybenzyl)azetidine-3-carboxylic acid;
1-(4-(2-methylbenzyloxy)benzyl)azetidine-3-carboxylic acid;
1-(4-(4-fluorobenzyloxy)-3-methoxybenzyl)azetidine-3-carboxylic acid;
1-(4-(benzyloxy)-3-ethoxybenzyl)azetidine-3-carboxylic acid;
1-(1-(biphenyl-4-yl)ethyl)azetidine-3-carboxylic acid;
1-(1-(4′-methylbiphenyl-4-yl)ethyl)azetidine-3-carboxylic acid;
1-(1-(4′-chlorobiphenyl-4-yl)ethyl)azetidine-3-carboxylic acid;
1-(1-(3′-methoxybiphenyl-4-yl)ethyl)azetidine-3-carboxylic acid;
1-(1-(3′-(trifluoromethyl)biphenyl-4-yl)ethyl)azetidine-3-carboxylic acid;
1-(4-(benzylthio)-3-nitrobenzyl)azetidine-3-carboxylic acid;
1-(4-(4-fluorobenzyloxy)benzyl)azetidine-3-carboxylic acid;
1-(4-(hex-1-ynyl)benzyl)azetidine-3-carboxylic acid;
1-(4-pentylbenzyl)azetidine-3-carboxylic acid;
1-(1-(4-(2-chloro-6-fluorobenzyloxy)benzyl)azetidine-3-carboxylic acid;
1-(1-(4-(benzyloxy)-3-chlorobenzyl)azetidine-3-carboxylic acid;
1-(4-(2-chlorobenzyloxy)benzyl)azetidine-3-carboxylic acid;
1-(1-(4-(4-(methoxycarbonyl)benzyloxy)benzyl)azetidine-3-carboxylic acid;
1-(1-(4-(3-fluorobenzyloxy)benzyl)azetidine-3-carboxylic acid;
1-(1-(4-(2,4-dichlorobenzyloxy)benzyl)azetidine-3-carboxylic acid;
1-(1-(4-(2-methylbenzyloxy)benzyl)azetidine-3-carboxylic acid;
1-(4-hexylbenzyl)azetidine-3-carboxylic acid;
1-(4-((trimethylsilyl)ethynyl)benzyl)azetidine-3-carboxylic acid;
1-(4-(benzyloxy)-2-methylbenzyl)azetidine-3-carboxylic acid;
1-(4-(benzyloxy)-3,5-dimethylbenzyl)azetidine-3-carboxylic acid;
1-(4-(4-bromobenzyloxy)benzyl)azetidine-3-carboxylic acid;
1-(4-(2-chloro-6-fluorobenzyloxy)benzyl)azetidine-3-carboxylic acid;
1-(4-(benzyloxy)-3-chlorobenzyl)azetidine-3-carboxylic acid;
1-(4-(3-(methoxycarbonyl)benzyloxy)benzyl)azetidine-3-carboxylic acid;
1-(4-(4-chlorobenzyloxy)-3-methoxybenzyl)azetidine-3-carboxylic acid;
1-(4-(2-chlorobenzyloxy)-3-methoxybenzyl)azetidine-3-carboxylic acid;
1-(3-methoxy-4-(4-methylbenzyloxy)benzyl)azetidine-3-carboxylic acid;
1-(4-(2-chlorobenzyloxy)-3-ethoxybenzyl)azetidine-3-carboxylic acid;
1-(4-(4-chlorobenzyloxy)benzyl)azetidine-3-carboxylic acid;
1-(4-(2-chloro-benzyloxy)benzyl)azetidine-3-carboxylic acid;
1-(4-(2-chloro-4-fluorobenzyloxy)benzyl)azetidine-3-carboxylic acid;
1-(4-(3-methylbenzyloxy)benzyl)azetidine-3-carboxylic acid;
1-(4-(3-(trifluoromethyl)benzyloxy)benzyl)azetidine-3-carboxylic acid;
1-(4-(3-methoxybenzyloxy)benzyl)azetidine-3-carboxylic acid;
1-(4-(3-bromobenzyloxy)benzyl)azetidine-3-carboxylic acid;
1-(4-(4-chlorobenzyloxy)-3-ethoxybenzyl)azetidine-3-carboxylic acid;
1-(4-(4-nitrobenzoyloxy)benzyl)azetidine-3-carboxylic acid;
1-(4-(4-fluorobenzoyloxy)-3-methoxybenzyl)azetidine-3-carboxylic acid;
1-(4-(2,4-dichlorobenzyloxy)-3-methoxybenzyl)azetidine-3-carboxylic acid;
1-(4-(benzyloxy)-3,5-dibromobenzyl)azetidine-3-carboxylic acid;
1-(4-(benzyloxy)-3-bromo-5-methoxybenzyl)azetidine-3-carboxylic acid;
1-(4-(benzyloxy)-3,5-dimethoxybenzyl)azetidine-3-carboxylic acid;
1-(4-(2-fluorobenzyloxy)benzyl)azetidine-3-carboxylic acid;
1-(4-(3-fluorobenzyloxy)benzyl)azetidine-3-carboxylic acid;
1-(4-(2,4-dichlorobenzyloxy)benzyl)azetidine-3-carboxylic acid;
1-(4-(2-methylbenzyloxy)benzyl)azetidine-3-carboxylic acid;
1-(4-(4-fluorobenzyloxy)-3-methoxybenzyl)azetidine-3-carboxylic acid;
1-(4-(2,4,6-trimethylbenzyloxy)benzyl)azetidine-3-carboxylic acid;
1-(4-(2-methoxy-2-oxo-1-phenylethoxy)benzyl)azetidine-3-carboxylic acid;
1-(4-(2-(methoxycarbonyl)-6-nitrobenzyloxy)benzyl)azetidine-3-carboxylic acid;
1-(4-(4-fluorobenzyloxy)-3-nitrobenzyl)azetidine-3-carboxylic acid;
1-(4-(3,4-dichlorobenzyloxy)-3-nitrobenzyl)azetidine-3-carboxylic acid;
1-(4-(benzyloxy)-3-ethoxybenzyl)azetidine-3-carboxylic acid;
1-(4-(3,4,5-trimethoxybenzoyloxy)benzyl)azetidine-3-carboxylic acid;
1-(4-(4-methylbenzyloxy)benzyl)azetidine-3-carboxylic acid;
1-(4-(3-chlorobenzyloxy)benzyl)azetidine-3-carboxylic acid;
1-(4-butoxybenzyl)azetidine-3-carboxylic acid;
1-(4-(pentyloxy)benzyl)azetidine-3-carboxylic acid;
1-(4-(isopentyloxy)benzyl)azetidine-3-carboxylic acid;
1-(4-pentylbenzyl)azetidine-3-carboxylic acid;
1-(4-(4-chlorophenoxy)benzyl)azetidine-3-carboxylic acid;
1-(4-butoxy-3-nitrobenzyl)azetidine-3-carboxylic acid;
1-(4-(2,4-dichlorophenoxy)benzyl)azetidine-3-carboxylic acid;
1-(4-(4-methoxyphenoxy)benzyl)azetidine-3-carboxylic acid;
1-(4-(4-bromophenoxy)benzyl)azetidine-3-carboxylic acid;
1-(4-(3-chlorophenoxy)benzyl)azetidine-3-carboxylic acid;
1-(4-(3,4-dimethylphenoxy)benzyl)azetidine-3-carboxylic acid;
1-(4-(4-tert-butylphenoxy)-3-nitrobenzyl)azetidine-3-carboxylic acid;
1-(4-(4-chloro-2-nitrophenoxy)benzyl)azetidine-3-carboxylic acid;
1-(4-(4-fluorophenoxy)-3-nitrobenzyl)azetidine-3-carboxylic acid;
1-(3-nitro-4-(3-(trifluoromethyl)phenoxy)benzyl)azetidine-3-carboxylic acid;
1-(3-nitro-4-(p-tolyloxy)benzyl)azetidine-3-carboxylic acid;
1-(4-(2,4-difluorophenoxy)-3-nitrobenzyl)azetidine-3-carboxylic acid;
1-(4-cyclopentyloxy)-3-methoxybenzyl)azetidine-3-carboxylic acid;
1-(4-(cyclopentyloxy)benzyl)azetidine-3-carboxylic acid;
1-(4-butoxy-3-methoxybenzyl)azetidine-3-carboxylic acid;
1-(4-(hexyloxy)benzyl)piperidine-4-carboxylic acid;
(S)-2-(1-(4-(hexyloxy)benzyl)pyrrolidin-2-yl)acetic acid;
(R)-1-(4-(hexyloxy)benzyl)pyrrolidine-3-carboxylic acid;
(R)-1-(4-(hexyloxy)benzyl)piperidine-3-carboxylic acid;
(S)-1-(4-(hexyloxy)benzyl)piperidine-3-carboxylic acid;
1-(4-(hexyloxy)benzyl)-3-methylpiperidine-4-carboxylic acid;
1-(4-(hexyloxy)benzyl)pyrrolidine-3-carboxylic acid;
(3R,4S)-1-(4-(hexyloxy)benzyl)pyrrolidine-3,4-dicarboxylic acid;
1-(4-phenoxybenzyl)azetidine-3-carboxylic acid;
1-(4-(benzyloxy)benzyl)azetidine-3-carboxylic acid;
1-(4-propoxybenzyl)azetidine-3-carboxylic acid;
1-(4-butoxybenzyl)azetidine-3-carboxylic acid;
1-(4-(4-tent-butylthiazol-2-yl)benzyl)azetidine-3-carboxylic acid;
1-(4-(benzyloxy)-3-methoxybenzyl)azetidine-3-carboxylic acid;
(E)-1-(4-styrylbenzyl)azetidine-3-carboxylic acid;
1-(4-(hexyloxy)benzyl)azetidine-3-carboxylic acid;
1-(4-butylbenzyl)azetidine-3-carboxylic acid;
1-(4-(allyloxy)benzyl)azetidine-3-carboxylic acid;
1-((2-fluorobiphenyl-4-yl)methyl)azetidine-3-carboxylic acid;
1-(4-(thiophen-2-yl)benzyl)azetidine-3-carboxylic acid;
1-((biphenyl-4-yl)methyl)azetidine-3-carboxylic acid;
1-(3,4-bis(benzyloxy)benzyl)azetidine-3-carboxylic acid;
1-(4-(benzyloxy)-2-methoxybenzyl)azetidine-3-carboxylic acid;
1-(4-isobutylbenzyl)azetidine-3-carboxylic acid;
1-((3′,4′-dichlorobiphenyl-4-yl)methyl)azetidine-3-carboxylic acid;
1-(4-(pentyloxy)benzyl)azetidine-3-carboxylic acid;
1-(3-ethoxy-4-(heptyloxy)benzyl)azetidine-3-carboxylic acid;
1-(4-(isopentyloxy)benzyl)azetidine-3-carboxylic acid;
1-(4-(2-(3,4-dimethylphenyl)-2-oxoethoxy)benzyl)azetidine-3-carboxylic acid;
1-(3-methoxy-4-(pentyloxy)benzyl)azetidine-3-carboxylic acid;
1-(4-butoxy-3-ethoxybenzyl)azetidine-3-carboxylic acid;
1-(3-bromo-5-methoxy-4-propoxybenzyl)azetidine-3-carboxylic acid;
1-(3-chloro-5-methoxy-4-propoxybenzyl)azetidine-3-carboxylic acid;
1-(4-isobutoxy-3-ethoxybenzyl)azetidine-3-carboxylic acid;
1-(4-(isopentyloxy)-3-methoxybenzyl)azetidine-3-carboxylic acid;
1-(4-(3-fluoropropoxy)benzyl)azetidine-3-carboxylic acid;
1-(4-((2-cyanothiophen-3-yl)methoxy)benzyl)azetidine-3-carboxylic acid;
1-((4′-ethylbiphenyl-4-yl)methyl)azetidine-3-carboxylic acid;
1-((2′-methoxybiphenyl-4-yl)methyl)azetidine-3-carboxylic acid;
1-((3′,5′-dichlorobiphenyl-4-yl)methyl)azetidine-3-carboxylic acid;
1-((3′-chlorobiphenyl-4-yl)methyl)azetidine-3-carboxylic acid;
1-((3′,4′-dimethylbiphenyl-4-yl)methyl)azetidine-3-carboxylic acid;
1-((3′-methylbiphenyl-4-yl)methyl)azetidine-3-carboxylic acid;
1-(4-(3,4-dichlorobenzyloxy)benzyl)azetidine-3-carboxylic acid;
1-(4-(4-chlorophenoxy)benzyl)azetidine-3-carboxylic acid;
1-(3′-(trifluoromethyl)biphenyl-4-yl)methyl)azetidine-3-carboxylic acid;
1-(4-(naphthalen-1-yl)benzyl)azetidine-3-carboxylic acid;
1-(4-(3,4-dichlorobenzyloxy)benzyl)-3-methylpiperidine-4-carboxylic acid;
1-(4-(3,4-dichlorobenzyloxy)benzyl)azetidine-3-carboxylic acid;
1-(4-(hexyloxycarbonyl)benzyl)azetidine-3-carboxylic acid;
1-(4-(hexyloxy)benzyl)-4-methylpyrrolidine-3-carboxylic acid;
1-(4-(3,4-dichlorobenzyloxy)-3-nitrobenzyl)azetidine-3-carboxylic acid;
1-(4-(hexyloxy)-3-methoxybenzyl)azetidine-3-carboxylic acid;
1-(4-(2-phenylacetyl)benzyl)azetidine-3-carboxylic acid;
1-(4-phenethylbenzyl)azetidine-3-carboxylic acid;
1-(4-(2-(3-(trifluoromethyl)phenyl)acetyl)benzyl)azetidine-3-carboxylic acid;
1-(4-(benzyloxy)-3-fluorobenzyl)azetidine-3-carboxylic acid;
1-(4-(benzyloxy)-2-chlorobenzyl)azetidine-3-carboxylic acid;
1-(4-(benzyloxy)-2-fluorobenzyl)azetidine-3-carboxylic acid;
1-(4-(benzyloxy)-3-chlorobenzyl)azetidine-3-carboxylic acid;
1-(3-fluoro-4-(3-(trifluoromethyl)benzyloxy)benzyl)azetidine-3-carboxylic acid;
1-(2-chloro-4-(3-(trifluoromethyl)benzyloxy)benzyl)azetidine-3-carboxylic acid;
1-(2-fluoro-4-(3-(trifluoromethyl)benzyloxy)benzyl)azetidine-3-carboxylic acid;
1-(3-chloro-4-(3-(trifluoromethyl)benzyloxy)benzyl)azetidine-3-carboxylic acid;
1-(4-(3,4-dichlorobenzyloxy)-3-fluorobenzyl)azetidine-3-carboxylic acid;
1-(4-(3,4-dichlorobenzyloxy)-2-fluorobenzyl)azetidine-3-carboxylic acid;
1-(4-(3,4-dichlorobenzyloxy)-2-methylbenzyl)azetidine-3-carboxylic acid;
1-(4-(benzyloxy)-2-methylbenzyl)azetidine-3-carboxylic acid;
1-(2-methyl-4-(3-(trifluoromethyl)benzyloxy)benzyl)azetidine-3-carboxylic acid;
1-(4-(1-phenylethoxy)benzyl)azetidine-3-carboxylic acid;
(R)-1-(4-(1-phenylethoxy)benzyl)azetidine-3-carboxylic acid;
(S)-1-(4-(1-phenylethoxy)benzyl)azetidine-3-carboxylic acid;
1-(4-(2-phenylacetyl)benzyl)pyrrolidine-3-carboxylic acid;
1-(4-(2-phenylacetyl)benzyl)pyrrolidine-3-carboxylic acid;
1-(4-(2-(3,4-dichlorophenyl)acetyl)benzyl)azetidine-3-carboxylic acid;
1-(4-(2-(3,4-dichlorophenyl)acetyl)phenyl)pyrrolidine-3-carboxylic acid;
1-(4-hexanoylbenzyl)azetidine-3-carboxylic acid;
1-(4-hexanoylbenzyl)pyrrolidine-3-carboxylic acid;
(1R,3S)-3-((6-hexanoylpyridin-3-yl)methylamino)cyclopentanecarboxylic acid;
1-(4-heptanoylbenzyl)azetidine-3-carboxylic acid;
1-(4-heptanoylbenzyl)pyrrolidine-3-carboxylic acid;
3-(4-heptanoylbenzyl)cyclopentanecarboxylic acid;
1-(4-(3,3-dimethylbut-1-ynyl)benzyl)azetidine-3-carboxylic acid;
1-(2-fluoro-4-(3-(trifluoromethyl)benzyloxy)benzyl)piperidine-4-carboxylic acid;
1-(2-fluoro-4-(3-(trifluoromethyl)benzyloxy)benzyl)piperidine-3-carboxylic acid;
3-(4-(benzyloxy)phenylamino)cyclopentanecarboxylic acid;
1-(1-(4-(benzyloxy)phenyl)ethyl)azetidine-3-carboxylic acid;
1-(4-((trimethylsilyl)ethynyl)benzyl)piperidine-4-carboxylic acid;
1-(4-((trimethylsilyl)ethynyl)benzyl)pyrrolidine-3-carboxylic acid;
1-(4-((trimethylsilyl)ethynyl)benzyl)piperidine-3-carboxylic acid;
4,4-dimethyl-1-(4-((trimethylsilyl)ethynyl)benzyl)pyrrolidine-3-carboxylic acid;
4-methyl-1-(4-((trimethylsilyl)ethynyl)benzyl)pyrrolidine-3-carboxylic acid;
1-(3′,5′-bis(trifluoromethyl)biphenyl-4-yl)methyl)azetidine-3-carboxylic acid;
1-(4-(5-(trifluoromethyl)pyridin-2-yl)benzyl)azetidine-3-carboxylic acid;
1-(4-(5-cyanopyridin-2-yl)benzyl)azetidine-3-carboxylic acid;
1-(4-(4-cyanopyridin-2-yl)benzyl)azetidine-3-carboxylic acid;
1-(4-(3-nitropyridin-2-yl)benzyl)azetidine-3-carboxylic acid;
1-(4-(benzo[d][1,3]dioxol-5-yl)benzyl)azetidine-3-carboxylic acid;
1-(4-chloro-3-fluorobenzyl)azetidine-3-carboxylic acid;

1-((9-methyl-9H-carbazol-2-yl)methyl)azetidine-3-carboxylic acid;
1-((3′-methoxybiphenyl-4-yl)methyl)azetidine-3-carboxylic acid;
1-(4′-(trifluoromethyl)biphenyl-4-yl)methyl)azetidine-3-carboxylic acid;
1-((9H-fluoren-3-yl)methyl)azetidine-3-carboxylic acid;
1-((2-fluorobiphenyl-4-yl)methyl)azetidine-3-carboxylic acid; or
1-(4-(phenylethynyl)benzyl)azetidine-3-carboxylic acid.

Another embodiment of the invention relates to a compound according to any of the foregoing embodiments wherein the compound is a compound of Formula (II)

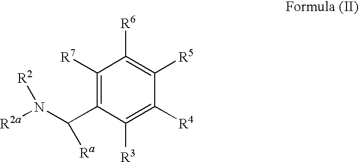

wherein

R³, R⁴, R⁶, and R⁷ are independently selected from the group consisting of optionally substituted alkenyl, optionally substituted alkoxy, optionally substituted alkoxycarbonyl, optionally substituted alkoxysulfonyl, optionally substituted alkyl, optionally substituted alkylcarbonyl, optionally substituted alkylcarbonyloxy, optionally substituted alkylsulfonyl, optionally substituted alkylthio, optionally substituted alkynyl, optionally substituted aryl, optionally substituted aryloxy, amido, optionally substituted amino, carboxy, cyano, formyl, halo, haloalkoxy, haloalkyl, hydrogen, hydroxyl, hydroxyalkyl, mercapto, nitro, silyl and silyloxy;

R⁵ is optionally substituted aryl, optionally substituted arylalkyl, optionally substituted arylalkylcarbonyl, optionally substituted 2-thiazolyl, optionally substituted arylalkoxy, optionally substituted arylalkylthio, optionally substituted arylcarbonyloxy, optionally substituted arylcarbonylalkoxy, optionally substituted aryloxycarbonyl, optionally substituted arylalkenyl, optionally substituted arylalkyl, optionally substituted alkyl, optionally substituted alkylcarbonyl, optionally substituted alkenyl, optionally substituted alkynyl, optionally substituted alkenyloxy, optionally substituted aryloxy, optionally substituted aryloxycarbonyl, optionally substituted alkoxy, optionally substituted alkoxycarbonyl, haloalkoxy, optionally substituted cycloalkoxy, optionally substituted alkenyloxy, optionally substituted arylalkynyl, optionally substituted cycloalkyl, optionally substituted cycloalkyloxy, optionally substituted heterarylalkyl or optionally substituted heteroaryl.

Another embodiment of the invention relates to a compound according to any of the foregoing embodiments wherein R² and R^{2a} are independently hydrogen, optionally substituted alkyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹.

Another embodiment of the invention relates to a compound according to any of the foregoing embodiments wherein R^{a} is hydrogen or optionally substituted alkyl.

Another embodiment of the invention relates to a compound according to any of the foregoing embodiments wherein R^{2a} is hydrogen, optionally substituted alkyl, optionally substituted alkoxyalkyl, optionally substituted cycloalkyl, optionally substituted cyclohexenyl, optionally substituted bridged cycloalkyl, or tetrahydrofuranyl.

Another embodiment of the invention relates to a compound according to any of the foregoing embodiments wherein the compound is
1-(3-(4-(hexyloxy)benzylamino)propyl)pyrrolidin-2-one;
(S)-2-(4-(hexyloxy)benzylamino)-3-methylbutan-1-ol;
(R)-2-(4-(hexyloxy)benzylamino)-3-methylbutan-1-ol;
(S)-1-(4-(hexyloxy)benzylamino)propan-2-ol;
(R)-2-(4-(hexyloxy)benzylamino)-3-methylbutan-1-ol;
(2R,3S)-3-(4-(hexyloxy)benzylamino)bicyclo[2.2.1]hept-5-ene-2-carboxylic acid;
(2S,3R)-3-(4-(hexyloxy)benzylamino)bicyclo[2.2.1]heptane-2-carboxylic acid;
(1R,6S)-6-(4-(hexyloxy)benzylamino)cyclohex-3-enecarboxylic acid;
(R)—N-(4-(hexyloxy)benzyl)-1-methoxypropan-2-amine;
3-((4-(hexyloxy)benzyl)(isopropyl)amino)propanoic acid;
(S)—N-(4-(hexyloxy)benzyl)tetrahydrofuran-3-amine;
N-(4-(hexyloxy)benzyl)-1-methoxybutan-2-amine;
2-(4-(hexyloxy)benzylamino)cycloheptanecarboxylic acid;
1-(4-(hexyloxy)benzylamino)-2-methylpropan-2-ol;
2-(4-(hexyloxy)benzylamino)cyclopentanecarboxylic acid;
(S)-2-(2-fluoro-4-(3-(trifluoromethyl)benzyloxy)benzylamino)-3-methylbutan-1-ol;
(R)—N-(2-fluoro-4-(3-(trifluoromethyl)benzyloxy)benzyl)tetrahydrofuran-3-amine;
(S)—N-(2-fluoro-4-(3-(trifluoromethyl)benzyloxy)benzyl)tetrahydrofuran-3-amine;
1-(2-fluoro-4-(3-(trifluoromethyl)benzyloxy)benzylamino)-2-methylpropan-2-ol;
(1-(2-fluoro-4-(3-(trifluoromethyl)benzyloxy)benzylamino)cyclopropyl)methanol;
1-(2-fluoro-4-(3-(trifluoromethyl)benzyloxy)benzylamino)cyclopropane carboxylic acid;
2-(2-fluoro-4-(3-(trifluoromethyl)benzyloxy)benzylamino)-2-methylpropanoic acid;
3-(2-fluoro-4-(3-(trifluoromethyl)benzyloxy)benzylamino)-2-methylpropanoic acid;
2-(2-fluoro-4-(3-(trifluoromethyl)benzyloxy)benzylamino)methyl)butan-1-ol;
N-(2-fluoro-4-(3-(trifluoromethyl)benzyloxy)benzyl)-3-methoxy-2-methylpropan-1-amine;
(R)-2-(2-fluoro-4-(3-(trifluoromethyl)benzyloxy)benzylamino)-3-methylbutan-1-ol;
(S)-1-(2-fluoro-4-(3-(trifluoromethyl)benzyloxy)benzylamino)propan-2-ol;
(R)-3-(2-fluoro-4-(3-(trifluoromethyl)benzyloxy)benzylamino)propane-1,2-diol;
(S)-3-(2-fluoro-4-(3-(trifluoromethyl)benzyloxy)benzylamino)propane-1,2-diol;
2-(2-fluoro-4-(3-(trifluoromethyl)benzyloxy)benzylamino)propane-1,3-diol;
2-(2-fluoro-4-(3-(trifluoromethyl)benzyloxy)benzylamino)-2-methylpropan-1-ol;
3-(2-fluoro-4-(3-(trifluoromethyl)benzyloxy)benzylamino)propane-1,2-diol;
(S)-1-(2-fluoro-4-(3-(trifluoromethyl)benzyloxy)benzylamino)propan-2-ol;
(S)-2-(2-fluoro-4-(3-(trifluoromethyl)benzyloxy)benzylamino)butan-1-ol;
1-(2-fluoro-4-(3-(trifluoromethyl)benzyloxy)benzyl)-4-methylpyrrolidine-3-carboxylic acid;
(R)-3-(4-((trimethylsilyl)ethynyl)benzylamino)propane-1,2-diol;
4-(4-((trimethylsilyl)ethynyl)benzylamino)butanoic acid;
(R)-2-(4-((trimethylsilyl)ethynyl)benzylamino)butanoic acid;
2-methyl-2-(4-((trimethylsilyl)ethynyl)benzylamino)propanoic acid;
2-methyl-3-(4-((trimethylsilyl)ethynyl)benzylamino)propanoic acid;
2-(4-((trimethylsilyl)ethynyl)benzylamino)propane-1,3-diol;
(S)-3-(4-((trimethylsilyl)ethynyl)benzylamino)propane-1,2-diol;
(R)-2-(4-((trimethylsilyl)ethynyl)benzylamino)propanoic acid;
(S)-2-hydroxy-3-(4-((trimethylsilyl)ethynyl)benzylamino)propanoic acid;
(S)-2-(4-((trimethylsilyl)ethynyl)benzylamino)butanoic acid;
2-(4-((trimethylsilyl)ethynyl)benzylamino)acetic acid;
3-(ethyl(4-((trimethylsilyl)ethynyl)benzyl)amino)propanoic acid;
(S)-2-(4-((trimethylsilyl)ethynyl)benzylamino)propanoic acid; or
(1R,3S)-3-(5-pentylpyrimidin-2-ylamino)cyclopentanecarboxylic acid.

Another embodiment of the invention relates to a compound according to any of the foregoing embodiments wherein the compound is 2-(2-fluoro-4-(3-(trifluoromethyl)benzyloxy)benzyl)octahydro cyclopenta[c]pyrrole-3a-carboxylic acid.

Another embodiment of the invention relates to a compound according to any of the foregoing embodiments wherein the compound is selective for the S1P₅ receptor and does not cause lymphopenia or immunosuppression at therapeutically relevant amounts of drug.

Another embodiment of the invention relates to a method for treating or preventing conditions, disorders or deficits modulated by S1P₅ in treating or preventing a condition or disorder selected from a neurodegenerative disorder, attention deficit disorder, attention deficit hyperactivity disorder (ADHD), substance abuse including alcohol abuse, bipolar disorder, mild cognitive impairment, age-associated memory impairment (AAMI), senile dementia, AIDS dementia, Pick's Disease, dementia associated with Lewy bodies, dementia associated with Down's syndrome, schizophrenia, schizoaffective disorder, smoking cessation, diminished CNS function associated with traumatic brain injury, infertility, lack of circulation, need for new blood vessel growth associated with wound healing, ischemia, sepsis, neurodegeneration, neuropathic pain, inflammation and inflammatory disorders comprising administering a therapeutically effective amount of S1P₅ receptor ligand or a compound of Formula (I), or a pharmaceutically acceptable salt, biologically active metabolite, solvate, hydrate, prodrug, enantiomer or stereoisomer thereof to the patient.

Another embodiment of the invention relates to a method of treating neurodegeneration, comprising the step of administering to a subject in need thereof a therapeutically effective amount of one or more compounds of any one of claims 1-41, or a pharmaceutically acceptable salt, biologically active metabolite, solvate, hydrate, prodrug, enantiomer or stereoisomer thereof.

Another embodiment of the invention relates to the foregoing method wherein said neurodegenerative disorder is selected from the group consisting of neurodegenerative diseases selected from Alzheimer's disease, Huntington's disease, Parkinson's disease, Amyotrophic Lateral Sclerosis, asphyxia, acute thromboembolic stroke, focal and global ischemia, and transient cerebral ischemic attacks.

Another embodiment of the invention relates to a method for use of treating or preventing a condition or disorder characterized by attention or cognitive dysfunction comprising administering a therapeutically effective amount of a S1P₅ ligands to a subject in need thereof in combination with a nicotinic acetylcholine receptor ligand or an acetylcholinesterase inhibitor comprising the step of administering to a subject in need thereof a therapeutically effective amount of one or more compounds of Formula (I),

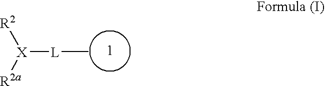

or a pharmaceutically acceptable salt, biologically active metabolite, solvate, hydrate, prodrug, enantiomer or stereoisomer thereof wherein

Ring 1 is optionally substituted aryl or optionally substituted heteroaryl;

L is —N(R^{a})—, —O— or C(R^{a})₂; wherein
R^{a} is independently H or optionally substituted alkyl;

X is N when L is C(R^{a})₂ or

X is CR^{a}; when L is —N— or —O—;

R² and R^{2a} are independently hydrogen, optionally substituted alkyl, optionally substituted alkenyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹; wherein
W is O or S; and
R¹¹ is —OR, —N(R)₂ or —SR; wherein
   R is independently hydrogen, optionally substituted alkyl or haloalkyl; or

when X is N or C, R² and R^{2a} together with the carbon or nitrogen atom to which they are attached form an optionally substituted cycloalkyl, optionally substituted azetidine, optionally substituted pyrrolidine, optionally substituted piperidine or optionally substituted octahydrocyclopenta[c]pyrrolyl ring, provided that the azetidine ring formed by R² and R^{2a} together with the carbon or nitrogen atom to which they are attached is not substituted by
one or more phenyl;
phenyl and OH;
phenyl and —N(H)C(CH₃)₃;
—CH₂—O-optionally substituted pyridinyl;
—NH-optionally substituted quinazolinyl;
—O-optionally substituted pyridinyl;
—O—Si(CH₃)₂—C(CH₃)₃;
—C(OH)(4-(trifluoromethoxy)phenyl)(4-methoxyphenyl);
—C(OH)(4-(trifluoromethoxy)phenyl)(4-methoxyphenyl) and oxo;
—NH-isoquinolinyl;
optionally substituted alkyl and optionally substituted dioxolanyl;
oxo and —O-alkenyl;
oxo, two F and optionally substituted phenyl;
optionally substituted alkenyl and —O—C(O)-optionally substituted phenyl;
provided that when Ring 1 is optionally substituted phenyl, L is CH₂ X is N or C, and R² and R^{2a} together with the carbon or nitrogen atom to which they are attached form an optionally substituted cycloalkyl, or optionally substituted azetidine, Ring 1 is not substituted by
   —CH═N—OCH₂CH₃;
   —Cl and —NH₂;
   —C(═O)CH₂CH₂-optionally substituted oxazolyl;
   —NH—C(O)-alkenyl-optionally substituted pyridinyl;
   —NO₂ and COOH—O-alkyl-optionally substituted oxazolyl;
   —O—CH₂-optionally substituted benzofuranyl;
   —O—CH₂-optionally substituted phenyl;
   —O—CH₂-optionally substituted pyrazolyl;
   —O—CH₂-optionally substituted thienyl;
   —O-optionally substituted (C₈)alkyl;
   —O-optionally substituted (C₈)alkyl and halo;
   —(C₆-C₁₂)alkyl wherein one or more carbons is optionally replaced by a nonperoxide oxygen;
   —(C₆-C₁₂)alkenyl wherein one or more carbons is optionally replaced by a nonperoxide oxygen;
   -pyrimidinyl substituted with oxo and —CF₂CF₃;
   -optionally substituted oxadiazole;
   -optionally substituted thiazolo[5,4-b]pyridine;
   -optionally substituted phenyl-CH₂—C(O)-optionally substituted pyrazolyl;
   -optionally substituted phenyl-CH₂—C(O)-optionally substituted thiazolyl;
   -optionally substituted phenyl-NH—C(O)-optionally substituted pyrazolyl;
   -optionally substituted phenyl-NH—C(O)-optionally substituted tetrazolyl;
   -optionally substituted phenyl-NH—C(O)-optionally substituted triazolyl;
   -optionally substituted pyridinyl-CH₂—C(O)-optionally substituted pyrazolyl;
   -optionally substituted pyridinyl-CH₂—C(O)-optionally substituted thiazolyl;
   -optionally substituted pyridinyl-NH—C(O)-optionally substituted pyrazolyl;
   -optionally substituted pyridinyl-NH—C(O)-optionally substituted tetrazoyl;
   -optionally substituted pyridinyl-NH—C(O)-optionally substituted triazolyl;
   -optionally substituted pyrimidinyl-CH₂—C(O)-optionally substituted pyrazolyl;
   -optionally substituted pyrimidinyl-NH—C(O)-optionally substituted pyrazolyl;
   -optionally substituted pyrimidinyl-NH—C(O)-optionally substituted triazolyl;
   -optionally substituted phenyl-CH₂—C(O)-optionally substituted triazolyl;
provided that when Ring 1 is optionally substituted isoxazolyl or optionally substituted oxazolyl, Ring 1 is not substituted by
   -optionally substituted phenyl-optionally substituted bicycle[2.2.1]heptanyl;
   -optionally substituted phenyl-optionally substituted alkyl-optionally substituted phenyl;
provided that when Ring 1 is optionally substituted pyridinyl, Ring 1 is not substituted by
   —C(O)—NH-optionally substituted phenyl;
   —O-optionally substituted phenyl; and
provided that when Ring 1 is optionally substituted phenyl or naphthyl, L is CH₂ and NR² and NR^{2a} form an optionally substituted pyrrolidine ring, the pyrrolidine ring is not substituted by
   —C(═O)(OH);
   —F and —C(═O)(OH);
   —OH and —C(═O)(OH);
   —P(═O)(OH)(OH);
   —OH and —P(═O)(OH)(OH);
   —CH₂C(═O)(OH); or
   tetrazolyl.

Another embodiment of the invention relates to a method according any of the foregoing embodiments, wherein said neuropathic pain is caused by peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain, pain associated with chronic alcoholism, hypothyroidism, uremia, multiple sclerosis, spinal cord injury, Parkinson's disease, epilepsy, vitamin deficiency, back pain, chronic low back pain, post-operative pain, injury-related pain, pain from spinal cord injury, eye pain, inflammatory pain, bone cancer pain, osteoarthritic pain, neuropathic pain, nociceptive pain, multiple sclerosis pain, post-stroke pain, diabetic neuropathic pain, neuropathic cancer pain, trigeminal neuralgia HIV-related neuropathic pain, phantom limb pain, fibromyalgia, or migraine.

Another embodiment of the invention relates to a method according any of the foregoing embodiments wherein said neurodegenerative disorder is selected from the group consisting of neurodegenerative diseases selected from Alzheimer's disease, age-associated memory impairment, senile dementia, AIDS dementia, Pick's disease, dementia associated with Lewy bodies, dementia associated with Down's syndrome, Huntington's disease, Parkinson's disease, Amyotrophic Lateral Sclerosis, mild cognitive disorders, asphyxia, acute thromboembolic stroke, diminished CNS function associated with traumatic brain injury, focal and global ischemia, and transient cerebral ischemic attacks.

Another embodiment of the invention relates to a method according any of the foregoing embodiments further comprising administering at least one additional therapeutic agent.

Another embodiment of the invention relates to a method for inhibiting lysophosphatidic acid receptors 1, 2 or 3 comprising the step of administering to a subject in need thereof a therapeutically effective amount of one or more compounds of Formula (I),

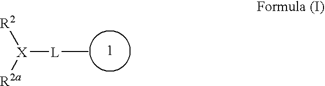

or a pharmaceutically acceptable salt, biologically active metabolite, solvate, hydrate, prodrug, enantiomer or stereoisomer thereof wherein
Ring 1 is optionally substituted aryl or optionally substituted heteroaryl;
L is —N(R^{a})—, —O— or C(R^{a})₂; wherein
   R^{a} is independently H or optionally substituted alkyl;
X is N when L is C(R^{a})₂ or
X is CR^{a}; when L is —N— or —O—;
R² and R^{2a} are independently hydrogen, optionally substituted alkyl, optionally substituted alkenyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹; wherein
   W is O or S; and
   R¹¹ is —OR, —N(R)₂ or —SR; wherein
      R is independently hydrogen, optionally substituted alkyl or haloalkyl; or
when X is N or C, R² and R^{2a} together with the carbon or nitrogen atom to which they are attached form an optionally substituted cycloalkyl, optionally substituted azetidine, optionally substituted pyrrolidine, optionally substituted piperidine or optionally substituted octahydrocyclopenta[c]pyrrolyl ring, provided that the azetidine ring formed by R² and R^{2a} together with the carbon or nitrogen atom to which they are attached is not substituted by
   one or more phenyl;
   phenyl and OH;
   phenyl and —N(H)C(CH₃)₃;
   —CH₂—O-optionally substituted pyridinyl;
   —NH-optionally substituted quinazolinyl;
   —O-optionally substituted pyridinyl;
   —O—Si(CH₃)₂—C(CH₃)₃;
   —C(OH)(4-(trifluoromethoxy)phenyl)(4-methoxyphenyl);
   —C(OH)(4-(trifluoromethoxy)phenyl)(4-methoxyphenyl) and oxo;
   —NH-isoquinolinyl;
   optionally substituted alkyl and optionally substituted dioxolanyl;
   oxo and —O-alkenyl;
   oxo, two F and optionally substituted phenyl;
   optionally substituted alkenyl and —O—C(O)-optionally substituted phenyl;
provided that when Ring 1 is optionally substituted phenyl, L is CH₂ X is N or C, and R² and R^{2a} together with the carbon or nitrogen atom to which they are attached form an optionally substituted cycloalkyl, or optionally substituted azetidine, Ring 1 is not substituted by
   —CH═N—OCH₂CH₃;
   —Cl and —NH₂;
   —C(═O)CH₂CH₂-optionally substituted oxazolyl;
   —NH—C(O)-alkenyl-optionally substituted pyridinyl;
   —NO₂ and COOH—O-alkyl-optionally substituted oxazolyl;
   —O—CH₂-optionally substituted benzofuranyl;
   —O—CH₂-optionally substituted phenyl;
   —O—CH₂-optionally substituted pyrazolyl;
   —O—CH₂-optionally substituted thienyl;
   —O-optionally substituted (C₈)alkyl;
   —O-optionally substituted (C₈)alkyl and halo;
   —(C₆-C₁₂)alkyl wherein one or more carbons is optionally replaced by a nonperoxide oxygen;
   —(C₆-C₁₂)alkenyl wherein one or more carbons is optionally replaced by a nonperoxide oxygen;
   -pyrimidinyl substituted with oxo and —CF₂CF₃;
   -optionally substituted oxadiazole;
   -optionally substituted thiazolo[5,4-b]pyridine;
   -optionally substituted phenyl-CH₂—C(O)-optionally substituted pyrazolyl;
   -optionally substituted phenyl-CH₂—C(O)-optionally substituted thiazolyl;
   -optionally substituted phenyl-NH—C(O)-optionally substituted pyrazolyl;
   -optionally substituted phenyl-NH—C(O)-optionally substituted tetrazolyl;
   -optionally substituted phenyl-NH—C(O)-optionally substituted triazolyl;
   -optionally substituted pyridinyl-CH₂—C(O)-optionally substituted pyrazolyl;
   -optionally substituted pyridinyl-CH₂—C(O)-optionally substituted thiazolyl;
   -optionally substituted pyridinyl-NH—C(O)-optionally substituted pyrazolyl;
   -optionally substituted pyridinyl-NH—C(O)-optionally substituted tetrazoyl;
   -optionally substituted pyridinyl-NH—C(O)-optionally substituted triazolyl;
   -optionally substituted pyrimidinyl-CH₂—C(O)-optionally substituted pyrazolyl;
   -optionally substituted pyrimidinyl-NH—C(O)-optionally substituted pyrazolyl;
   -optionally substituted pyrimidinyl-NH—C(O)-optionally substituted triazolyl;
   -optionally substituted phenyl-CH₂—C(O)-optionally substituted triazolyl;
provided that when Ring 1 is optionally substituted isoxazolyl or optionally substituted oxazolyl, Ring 1 is not substituted by
   -optionally substituted phenyl-optionally substituted bicyclo[2.2.1]heptanyl;
   -optionally substituted phenyl-optionally substituted alkyl-optionally substituted phenyl;
provided that when Ring 1 is optionally substituted pyridinyl, Ring 1 is not substituted by
   —C(O)—NH-optionally substituted phenyl;
   —O-optionally substituted phenyl; and
provided that when Ring 1 is optionally substituted phenyl or naphthyl, L is CH₂ and NR² and NR^{2a} form an optionally substituted pyrrolidine ring, the pyrrolidine ring is not substituted by
   —C(═O)(OH);
   —F and —C(═O)(OH);
   —OH and —C(═O)(OH);
   —P(═O)(OH)(OH);
   —OH and —P(═O)(OH)(OH);
   —CH₂C(═O)(OH); or
   tetrazolyl.

In another embodiment the invention provides a method according to any of the foregoing methods further comprising administering at least one additional therapeutic agent.

In another embodiment the invention provides a method according to any of the foregoing methods wherein the at least one additional therapeutic agent is administered simultaneously with said one or more compounds of any one of claims 1-41, or a pharmaceutically acceptable salt, biologically active metabolite, solvate, hydrate, prodrug, enantiomer or stereoisomer thereof.

In another embodiment the invention provides a method according to any of the foregoing methods wherein the at least one additional therapeutic agent is administered sequentially with said one or more compounds of any one of claims 1-41, or a pharmaceutically acceptable salt, biologically active metabolite, solvate, hydrate, prodrug, enantiomer or stereoisomer thereof.

In another embodiment the invention provides a method according to any of the foregoing methods wherein the at least one additional therapeutic agent is selected from the group consisting of Bromocriptine, Pramipexole, Ropinirole, Amantadine, Levodopa, Selegiline, Benztropine, Sumatriptan, Phenyloin, Carbamazapine, Lamotrigine, Gabapentin, Topiramate, Phenobarbitol, Valproic acid, Diazepam, Lorazepam, Triazolam, Oxazepam, Chlordiazepoxide, Phenobarbitol, Thiopental, Secobarbital, Acetylsalicylic Acid, Celecoxib, Diclofenac Sodium, Misoprostol, Diflunisal, Flurbiprofen, Ibuprofen, Indomethacin, Ketoprofen, Mefenamic Acid, Meloxicam, Naproxen, Naproxen Sodium, Piroxicam, Sulindac, Tiaprofenic Acid, Acetaminophen, Caffeine Citrate, Codeine Monohydrate, Codeine Sulfate Trihydrate, Codeine Phosphate, Fentanyl, Hydromorphone Hydrochloride, Meperidine Hydrochloride, Morphine Hydrochloride, Morphine Sulfate, Oxycodone Hydrochloride, Pentazocine Hydrochloride, Pentazocine Lactate, Floctafenine, Phenobarbital, Primidone, Clonazepam, Phenyloin, Ethosuximide, Methsuximide, Carbamazepine, Divalproex Sodium, Gabapentin, Lamotrigine, Levetiracetam, Topiramate, Valproate Sodium, Valproic Acid, Vigabatrin, Amitriptyline Hydrochloride, Bupropion Hydrochloride (Wellbutrin), Bupropion Hydrochloride (Zyban), Citalopram, Clomipramine Hydrochloride, DeS1Pramine Hydrochloride, Doxepin Hydrochloride, Fluoxetine Hydrochloride, Fluvoxamine Maleate, Imipramine Hydrochloride, Maprotiline Hydrochloride, Mirtazapine, Moclobemide, Nortriptyline Hydrochloride, Paroxetine Hydrochloride, Phenelzine Sulfate, Sertraline, Tranylcypromine Sulfate, Trazodone Hydrochloride, Trimipramine Maleate, Venlafaxine Hydrochloride, Chlorpromazine, Clozapine, Flupenthixol Decanoate, Flupenthixol Dihydrochloride, Fluphenazine Decanoate, Fluphenazine Hydrochloride, Haloperidol, Haloperidol Decanoate, Loxapine Hydrochloride, Loxapine Succinate, Methotrimeprazine, Olanzapine, Pericyazine, Perphenazine, Pimozide, Pipotiazine Palmitate, Prochlorperazine, Quetiapine Fumarate, Risperidone, Thioproperazine Mesylate, Thiothixene, Trifluoperazine Hydrochloride, Dextroamphetamine Sulfate, Methylphenidate Hydrochloride, Modafinil, Alprazolam, Bromazepam, Clobazam, Diazepam, Lorazepam, Nitrazepam, Oxazepam, Temazepam, Triazolam, Hydroxyzine Hydrochloride, Lithium Carbonate, Lithium Citrate, Almotriptan Malate, Naratriptan Hydrochloride, Rizatriptan, Sumatriptan Hemisulfate, Sumatriptan Succinate, Zolmitriptan, Entacapone, Levodopa/Benzerazide, Levodopa/Carbidopa, Pizotyline Hydrogen Malate, Pramipexole Dihydrochloride, Selegiline Hydrochloride, acetylcholine, carbamylcholine, bethanecol, pilocarpine, atropine, scopolamine, quaternary amines (methylatropine), nicotine, hexamethonium, mecamylamine, d-tubocurarine, succinylcholine, endrophonium, neostigmine and pyridostigmine, physostigmine, donepezil, echothiophate, pralidoxime, Dantrolene, Botulinum toxins, Norepinephrine, Epinephrine, phenylepherine, axymetazoline, tetrahydrozoline clonidine, methyldopa, isoproterenol, albuterol, terbutaline, salmeterol, ritodrine, Tyramine, Ephedrine, Pseudoephedrine, Amphetamine, methamphetamine, phenoxybenzamine(haloalkylamine), phentolamine(imidazoline), prozasin, tamsulosin(alpha 1A), propranolol, atenolol and pindolol.

In another embodiment the invention relates to a compound of the foregoing embodiment, wherein R¹ is hydrogen.

In another embodiment the invention relates to a compound of any of the foregoing embodiments, wherein R² is hydrogen.

In another embodiment the invention relates to a compound of any of the foregoing embodiments wherein R² is methyl.

In another embodiment the invention relates to a compound of any of the foregoing embodiments wherein R³, R⁴, R⁶, and R⁷ are independently selected from the group consisting of alkoxy, alkyl, halo, hydrogen and nitro.

In another embodiment the invention relates to a compound of any of the foregoing embodiments, wherein R³, R⁴, R⁶, and R⁷ are independently selected from the group consisting of methoxy, ethoxy, chloro, fluoro, bromo, hydrogen and nitro.

In another embodiment the invention relates to a compound of any of the foregoing embodiments, wherein R³ is hydrogen, fluoro, or methyl.

In another embodiment the invention relates to a compound of any of the foregoing embodiments wherein R³ is hydrogen.

In another embodiment the invention relates to a compound of any of the foregoing embodiments wherein R⁴ is hydrogen, nitro, methoxy, ethoxy, chloro, methyl, bromo, or fluoro.

In another embodiment the invention relates to a compound of any of the foregoing embodiments wherein R⁴ is hydrogen.

In another embodiment the invention relates to a compound of any of the foregoing embodiments wherein R⁶ is hydrogen, methyl, methoxy, or bromo.

In another embodiment the invention relates to a compound of any of the foregoing embodiments wherein R⁶ is hydrogen,

In another embodiment the invention relates to a compound of any of the foregoing embodiments wherein R⁷ is hydrogen.

In another embodiment the invention relates to a compound of any of the foregoing embodiments wherein R⁵ is —C₆(R⁸)₅; and R⁸ is independently selected for each occurrence from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydrogen, hydroxyl, hydroxyalkyl, mercapto, nitro, silyl and silyloxy.

In another embodiment the invention relates to a compound of any of the foregoing embodiments wherein R⁸ is independently selected for each occurrence from the group consisting of hydrogen, alkyl, alkoxy, haloalkyl, or halo.

In another embodiment the invention relates to a compound of any of the foregoing embodiments wherein R⁸ is independently selected for each occurrence from the group consisting of hydrogen, methyl, ethyl, methoxy, trifluoromethyl, bromo or chloro.

In another embodiment the invention relates to a compound of any of the foregoing embodiments wherein R⁵ is phenyl, 4-methylphenyl, 4-chlorophenyl, 3-methoxyphenyl, 3-trifluoromethylphenyl, 3,4-dichlorophenyl, 2-methoxyphenyl, 4-ethylphenyl, 3,5-dichlorophenyl, 3,4-dimethylphenyl, 3-methylphenyl, 4-bromophenyl, or 4-trifluoromethylphenyl.

In another embodiment the invention relates to a compound of any of the foregoing embodiments wherein R⁵ is —XCH₂C₆(R⁸)₅; X is O or S; and R⁸ is independently selected for each occurrence from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydrogen, hydroxyl, hydroxyalkyl, mercapto, nitro, silyl and silyloxy.

In another embodiment the invention relates to a compound of any of the foregoing embodiments wherein X is O.

In another embodiment the invention relates to a compound of any of the foregoing embodiments wherein X is S.

In another embodiment the invention relates to a compound of any of the foregoing embodiments wherein R⁸ is independently selected for each occurrence from the group consisting of hydrogen, halo, alkyl, alkoxy, alkoxycarbonyl, haloalkyl and nitro.

In another embodiment the invention relates to a compound of any of the foregoing embodiments wherein R⁸ is independently selected for each occurrence from the group consisting of hydrogen, chloro, fluoro, bromo, methyl, methoxy, methoxycarbonyl, trifluoromethyl and nitro.

In another embodiment the invention relates to a compound of any of the foregoing embodiments wherein R⁵ is phenylmethoxy, phenylmethylthio, 2-chlorophenylmethoxy, 4-chlorophenylmethoxy, 2-methylphenylmethoxy, 4-fluorophenylmethoxy, 4-(methyoxycarbonyl)-phenylmethoxy, 3-fluorophenylmethoxy, 2,4-dichlorophenyl-methoxy, 6-chloro-2-fluorophenylmethoxy, 2-chloro-4-fluorophenylmethoxy, 3-methylphenylmethoxy, 3-trifluoromethylphenylmethoxy, 3-methyoxyphenylmethoxy, 4-bromophenylmethoxy, 3-bromophenylmethoxy, 3-(methyoxycarbonyl)-phenylmethoxy, 2-fluorophenylmethoxy, 6-(methyoxycarbonyl)phenyl-2-nitrophenylmethoxy, 2,4,6-trimethylphenylmethoxy, 3,4-dichlorophenylmethoxy, 3,4,5-trimethoxyphenyl-methoxy, 3-nitrophenylmethoxy or 3,4-dimethylphenylmethoxy.

In another embodiment the invention relates to a compound of any of the foregoing embodiments wherein R⁵ is alkyl.

In another embodiment the invention relates to a compound of any of the foregoing embodiments wherein R⁵ is C₃-C₆ alkyl.

In another embodiment the invention relates to a compound of any of the foregoing embodiments wherein R⁵ is hexyl, pentyl, butyl, or i-propyl.

In another embodiment the invention relates to a compound of any of the foregoing embodiments wherein R⁵ is or —C(═O)R⁹; and R⁹ is alkyl.

In another embodiment the invention relates to a compound of any of the foregoing embodiments wherein R⁹ is C₄-C₈ alkyl.

In another embodiment the invention relates to a compound of any of the foregoing embodiments wherein R⁹ is pentyl or hexyl.

In another embodiment the invention relates to a compound of any of the foregoing embodiments wherein R⁵ is —C(═O)CH₂C₆(R⁸)₅; and R⁸ is independently selected for each occurrence from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydrogen, hydroxyl, hydroxyalkyl, mercapto, nitro, silyl and silyloxy.

In another embodiment the invention relates to a compound of any of the foregoing embodiments wherein R⁸ is hydrogen or halo.

In another embodiment the invention relates to a compound of any of the foregoing embodiments wherein R⁸ is hydrogen or chloro.

In another embodiment the invention relates to a compound of any of the foregoing embodiments wherein R⁵ is phenylmethylcarbonyl or 3,4-dichlorophenylmethylcarbonyl.

In another embodiment the invention relates to a compound of any of the foregoing embodiments wherein R⁵ is —OC₆(R⁸)₅; and R⁸ is independently selected for each occurrence from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydrogen, hydroxyl, hydroxyalkyl, mercapto, nitro, silyl and silyloxy.

In another embodiment the invention relates to a compound of any of the foregoing embodiments wherein R⁸ is hydrogen, halogen, alkyl, alkoxy, and haloalkyl.

In another embodiment the invention relates to a compound of any of the foregoing embodiments wherein R⁸ is hydrogen, chloro, methyl, methoxy, trifluoromethyl, fluoro, t-butyl, and bromo.

In another embodiment the invention relates to a compound of any of the foregoing embodiments wherein R⁵ is phenyloxy, 4-chlorophenyloxy, 2,4-dichlorophenyloxy, 4-methyoxyphenyloxy, 4-bromophenyloxy, 4-t-butylphenyloxy, 3,4-dimethylphenyloxy, 3, chlorophenyloxy, 2,4-difluorophenyloxy, 3-trifluorophenyloxy, or 4-chlorophenyloxy.

In another embodiment the invention relates to a compound of any of the foregoing embodiments wherein R⁵ is —OR⁹; and R⁹ is alkyl.

In another embodiment the invention relates to a compound of any of the foregoing embodiments wherein R⁹ is C₂-C₈ alkyl.

In another embodiment the invention relates to a compound of any of the foregoing embodiments, wherein R⁹ is heptyl, hexyl, pentyl, i-pentyl, butyl, i-butyl, propyl, or 3-fluoropentyl.

In another embodiment the invention relates to a method for measuring S1P₅ in a sample, comprising the steps of: administering a detectable quantity of an imaging agent according to any one of foregoing embodiments; and detecting the binding of the imaging agent to S1P₅ in the sample.

In another embodiment the invention relates to the foregoing method for measuring S1P₅ in a subject, comprising the steps of: administering a detectable quantity of an imaging agent according to any one of the foregoing embodiments and detecting the binding of the imaging agent to S1P₅ in the subject.

In another embodiment, the invention as embodied in a kit for imaging comprises a radioimaging agent or a fluorescence imaging agent, as described above, in combination with a pharmaceutically acceptable carrier such as human serum albumin. Human serum albumin for use in the kit of the invention may be made in any way, for example, through purification of the protein from human serum or though recombinant expression of a vector containing a gene encoding human serum albumin. Other substances may also be used as carriers in accordance with this embodiment of the invention, for example, detergents, dilute alcohols, carbohydrates, auxiliary molecules, and the like. The kit of the invention may of course also contain such other items as may facilitate its use, such as syringes, instructions, reaction vials, and the like.

In another embodiment the invention relates to, a kit according to the invention contains a radionuclide-labeled or fluorophore-labeled S1P₅ agonist or antagonist, as described herein, in combination with a pharmaceutically-acceptable carrier. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

### Combination Therapy

In one aspect of the invention, a compound of the invention, or a pharmaceutically acceptable salt, biologically active metabolite, solvate, hydrate, prodrug, enantiomer or stereoisomer thereof, can be used alone or in combination with another therapeutic agent to treat such diseases as those described above. It should be understood that the compounds of the invention can be used alone or in combination with an additional agent, e.g., a therapeutic agent, said additional agent being selected by the skilled artisan for its intended purpose. For example, the additional agent can be a therapeutic agent that is art-recognized as being useful to treat the disease or condition being treated by the compound of the present invention. The additional agent also can be an agent that imparts a beneficial attribute to the therapeutic composition e.g., an agent that affects the viscosity of the composition.

The combination therapy contemplated by the invention includes, for example, administration of a compound of the invention, or a pharmaceutically acceptable salt, biologically active metabolite, solvate, hydrate, prodrug, enantiomer or stereoisomer thereof, and additional agent(s) in a single pharmaceutical formulation as well as administration of a compound of the invention, or a pharmaceutically acceptable salt, biologically active metabolite, solvate, hydrate, prodrug, enantiomer or stereoisomer thereof, and additional agent(s) in separate pharmaceutical formulations. In other words, co-administration shall mean the administration of at least two agents to a subject so as to provide the beneficial effects of the combination of both agents. For example, the agents may be administered simultaneously or sequentially over a period of time.

It should further be understood that the combinations included within the invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

In certain embodiments, combinations comprise non-steroidal anti-inflammatory drug(s) also referred to as NSAIDS which include drugs like ibuprofen. Other combinations comprise corticosteroids including prednisolone; the well known side-effects of steroid use can be reduced or even eliminated by tapering the steroid dose required when treating patients in combination with the S1P5 modulators of this invention.

Non-limiting examples of therapeutic agents for rheumatoid arthritis with which a compound of the invention of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. S1P receptor modulators of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

In certain embodiments, combinations of therapeutic agents may interfere at different points in the autoimmune and subsequent inflammatory cascade; examples include TNF antagonists like chimeric, humanized or human TNF antibodies, D2E7 (HUMIRA™), (U.S. Pat. No. 6,090,382; incorporated by reference), CA2 (REMICADE™), CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1RA etc.) may be effective for the same reason. Other combinations include Interleukin 11. Yet other combinations are the other key players of the autoimmune response which may act parallel to, dependent on or in concert with IL-18 function; or IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another combination are non-depleting anti-CD4 inhibitors. Yet other combinations include antagonists of the co-stimulatory pathway CD80 (B7.1) or CD86 (B7.2) including antibodies, soluble receptors or antagonistic ligands.

A compound of the invention of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, colchicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., IRAK, NIK, IKK, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g., IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. In certain embodiments, combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of the invention of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g., IRAK, NIK, IKK, or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g., soluble p55 or p75 TNF receptors, sIL-1RI, sIL-1RII, sIL-6R) and antiinflammatory cytokines (e.g., IL-4, IL-10, IL-11, IL-13 and TGFβ). Examples of therapeutic agents for Crohn's disease with which a compound of the invention can be combined include the following: TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (Lenercept™)) inhibitors and PDE4 inhibitors. A compound of the invention can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors 6-mercaptopurines; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of the invention can be combined include the following: corticosteroids; prednisolone; methylprednisolone; azathioprine; cyclophosphamide; cyclosporine; methotrexate; 4-aminopyridine; tizanidine; interferon-β1a (Avonex®; Biogen); interferon-β1b (Betaseron®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; Copaxone®; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of the invention can be combined with antibodies to cell surface molecules such as CD2, CD3, CD4, CD8, CD19, CD20, CD25, CD28, CD30, CD40, CD45, CD69, CD80, CD86, CD90 or their ligands. A compound of the invention may also be combined with agents such as methotrexate, cyclosporine, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signalling by proinflammatory cytokines such as TNFα or IL-1 (e.g., IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, sIL-1RI, sIL-1RII, sIL-6R) and antiinflammatory cytokines (e.g., IL-4, IL-10, IL-13 and TGFβ).

Examples of therapeutic agents for multiple sclerosis in which a compound of the invention can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of the invention may also be combined with agents, such as alemtuzumab, dronabinol, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Central nervous system medications are used to treat the effects of a wide variety of medical conditions, including Alzheimer's disease, depression, and Parkinson's disease. This category of medication also includes analgesics (pain medications), sedatives, and anticonvulsants. Non-limiting examples of therapeutic agents for the treatment of disorders of the central nervous system with which a compound of the invention of the invention can be combined include the following: Bromocriptine, Pramipexole, Ropinirole, Amantadine, Levodopa, Selegiline, Benztropine, Sumatriptan, Phenyloin, Carbamazapine, Lamotrigine, Gabapentin, Topiramate, Phenobarbitol, Valproic acid, Diazepam, Lorazepam, Triazolam, Oxazepam, Chlordiazepoxide, Phenobarbitol, Thiopental, and Secobarbital.

A compound of the invention may also be combined with nonsteroidal anti-inflammatory agents, such as: Acetylsalicylic Acid, Celecoxib, Diclofenac Sodium, Misoprostol, Diflunisal, Flurbiprofen, Ibuprofen, Indomethacin, Ketoprofen, Mefenamic Acid, Meloxicam, Naproxen, Naproxen Sodium, Piroxicam, Sulindac, Tiaprofenic Acid

A compound of the invention may also be combined with opiate agonists, such as: Acetaminophen, Acetylsalicylic Acid, Caffeine Citrate, Codeine Monohydrate, Codeine Sulfate Trihydrate, Codeine Phosphate, Fentanyl, Hydromorphone Hydrochloride, Meperidine Hydrochloride, Morphine Hydrochloride, Morphine Sulfate and Oxycodone Hydrochloride.

A compound of the invention may also be combined with opiate partial agonists, such as: Pentazocine Hydrochloride and Pentazocine Lactate.

A compound of the invention may also be combined with analgesics and antipyretics, such as: Acetaminophen and Floctafenine.

A compound of the invention may also be combined with anticonvulsants, such as: Phenobarbital, Primidone, Clonazepam, Phenyloin, Ethosuximide, Methsuximide, Carbamazepine, Divalproex Sodium, Gabapentin, Lamotrigine, Levetiracetam, Topiramate, Valproate Sodium, Valproic Acid and Vigabatrin.

A compound of the invention may also be combined with antidepressants, such as: Amitriptyline Hydrochloride, Bupropion Hydrochloride (Wellbutrin), Bupropion Hydrochloride (Zyban), Citalopram, Clomipramine Hydrochloride, DeS1Pramine Hydrochloride, Doxepin Hydrochloride, Fluoxetine Hydrochloride, Fluvoxamine Maleate, Imipramine Hydrochloride, Maprotiline Hydrochloride, Mirtazapine, Moclobemide, Nortriptyline Hydrochloride, Paroxetine Hydrochloride, Phenelzine Sulfate, Sertraline, Tranylcypromine Sulfate, Trazodone Hydrochloride, Trimipramine Maleate, and Venlafaxine Hydrochloride.

A compound of the invention may also be combined with antipsychotic agents, such as: Chlorpromazine, Clozapine, Flupenthixol Decanoate, Flupenthixol Dihydrochloride, Fluphenazine Decanoate, Fluphenazine Hydrochloride, Haloperidol, Haloperidol Decanoate, Loxapine Hydrochloride, Loxapine Succinate, Methotrimeprazine, Olanzapine, Pericyazine, Perphenazine, Pimozide, Pipotiazine Palmitate, Prochlorperazine, Quetiapine Fumarate, Risperidone, Thioproperazine Mesylate, Thiothixene and Trifluoperazine Hydrochloride.

A compound of the invention may also be combined with amphetamines, such as: Dextroamphetamine Sulfate.

A compound of the invention may also be combined with anorexigenic agents and respiratory and cerebral stimulants, such as: Methylphenidate Hydrochloride and Modafinil.

A compound of the invention may also be combined with anxiolytics, sedatives and hypnotics, such as: Alprazolam, Bromazepam, Clobazam, Diazepam, Lorazepam, Nitrazepam, Oxazepam, Temazepam, Triazolam and Hydroxyzine Hydrochloride.

A compound of the invention may also be combined with antimanic agents, such as: Lithium Carbonate and Lithium Citrate.

A compound of the invention may also be combined with selective serotonin agonists, such as: Almotriptan Malate, Naratriptan Hydrochloride, Rizatriptan, Sumatriptan Hemisulfate, Sumatriptan Succinate and Zolmitriptan.

A compound of the invention may also be combined with central nervous system agents, such as: Entacapone, Levodopa/Benzerazide, Levodopa/Carbidopa, Pizotyline Hydrogen Malate, Pramipexole Dihydrochloride and Selegiline Hydrochloride.

The peripheral nervous system includes all nerves not in the brain or spinal cord and connects all parts of the body to the central nervous system. The peripheral (sensory) nervous system receives stimuli, the central nervous system interprets them, and then the peripheral (motor) nervous system initiates responses. A compound of the invention may also be combined with peripheral nervous system agents, such as acetylcholine, carbamylcholine, bethanecol, pilocarpine, atropine, scopolamine, quaternary amines (methylatropine), nicotine, hexamethonium, mecamylamine, d-tubocurarine, succinylcholine, endrophonium, neostigmine and pyridostigmine, physostigmine, donepezil, echothiophate, pralidoxime, Dantrolene, Botulinum toxins, Norepinephrine, Epinephrine, phenylepherine, axymetazoline, tetrahydrozoline clonidine, methyldopa, isoproterenol, albuterol, terbutaline, salmeterol, ritodrine, Tyramine, Ephedrine, Pseudoephedrine, Amphetamine, methamphetamine, phenoxybenzamine(haloalkylamine), phentolamine(imidazoline), prozasin, tamsulosin(alpha 1A), propranolol, atenolol and pindolol.

Non-limiting examples of therapeutic agents for angina with which a compound of the invention of the invention can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil HCl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril and bisoprolol fumarate.

Non-limiting examples of therapeutic agents for ankylosing spondylitis with which a compound of the invention can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, etanercept, D2E7 (U.S. Pat. No. 6,090,382; HUMIRA™) and infliximab.

Non-limiting examples of therapeutic agents for asthma with which a compound of the invention can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone and metaproterenol sulfate.

Non-limiting examples of therapeutic agents for COPD with which a compound of the invention can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast, and roflumilast.

Non-limiting examples of therapeutic agents for HCV with which a compound of the invention can be combined include the following: Interferon-alpha-2a, Interferon-alpha-2b, Interferon-alpha con1, Interferon-alpha-n1, pegylated interferon-alpha-2a, pegylated interferon-alpha-2b, ribavirin, peginterferon alfa-2b+ribavirin, ursodeoxycholic acid, glycyrrhizic acid, thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of the invention can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-gamma-1β.

Non-limiting examples of therapeutic agents for myocardial infarction with which a compound of the invention can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril HCl/mag carb, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of the invention can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, D2E7 (U.S. Pat. No. 6,090,382; HUMIRA™) and sulfasalazine.

Non-limiting examples of therapeutic agents for psoriatic arthritis with which a compound of the invention can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (U.S. Pat. No. 6,090,382; HUMIRA™) and efalizumab.

Non-limiting examples of therapeutic agents for restenosis with which a compound of the invention can be combined include the following: sirolimus, paclitaxel, everolimus, tacrolimus, ABT-578, and acetaminophen.

Non-limiting examples of therapeutic agents for sciatica with which a compound of the invention can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol HCl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene napsylate/apap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, and temazepam.

Examples of therapeutic agents for SLE (Lupus) with which a compound of the invention can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of the invention may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of the invention may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of the invention can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of the invention may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (U.S. Pat. No. 6,090,382; HUMIRA™), CA2 (REMICADE™), and CDP.

### DEFINITIONS

In this invention, the following definitions are applicable:

A “therapeutically effective amount” is an amount of a compound of the invention or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Physiologically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g., (+) or (−)-tartaric acid or mixtures thereof), amino acids (e.g., (+) or (−)-amino acids or mixtures thereof), and the like. These salts can be prepared by methods known to those skilled in the art.

Certain compounds of the invention which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of the invention and their salts may exist in more than one crystal form and the present invention includes each crystal form and mixtures thereof.

Certain compounds of the invention and their salts may also exist in the form of solvates, for example hydrates, and the present invention includes each solvate and mixtures thereof.

Certain compounds of the invention may contain one or more chiral centers, and exist in different optically active forms. When compounds of the invention contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step may be used to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of the invention contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of the invention and mixtures thereof.

Certain compounds of the invention may exist in different tautomeric forms or as different geometric isomers, and the present invention includes each tautomer and/or geometric isomer of compounds of the invention and mixtures thereof.

Certain compounds of the invention may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of the invention and mixtures thereof.

Certain compounds of the invention may exist in zwitterionic form and the present invention includes each zwitterionic form of compounds of the invention and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial. Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —C(O)₂H or a moiety that contains a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of a compound of the invention wherein the free hydrogen of a hydroxyl substituent is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyl-oxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, —P(O)(OH)₂, —P(O)(O(C₁-C₆)alkyl)₂ or glycosyl (the radical resulting from detachment of the hydroxyl of the hemiacetal of a carbohydrate).

For purposes of this invention, the chemical elements are identified in accordance with the Periodic Table of the Elements, CAS version, Handbook of Chemistry and Physics, 67th Ed., 1986-87, inside cover.

The articles “a” and “an” are used herein to refer to one or to more than one (i.e., to at least one) of the grammatical object of the article. By way of example, “an element” means one element or more than one element.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “arylalkoxy” and “heteroalkoxy” as used herein, means an aryl group or heteroaryl group, as defined herein, appended to the parent molecular moiety through an alkoxy group, as defined herein. Representative examples of arylalkoxy include, but are not limited to, 2-chlorophenylmethoxy, 3-trifluoromethylethoxy, and 2,3-methylmethoxy.

The term “arylalkyl” as used herein, means an aryl group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tent-butyl, n-pentyl, isopentyl, neopentyl, and n-hexyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” and “arylcarbonyloxy” as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio. The terms “arylthio,” “alkenylthio” and “arylakylthio,” for example, are likewise defined.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “amino” as used herein, refers to radicals of both unsubstituted and substituted amines appended to the parent molecular moiety through a nitrogen atom. The two groups are each independently hydrogen, alkyl, alkylcarbonyl, alkylsulfonyl, arylcarbonyl, or formyl. Representative examples include, but are not limited to methylamino, acetylamino, and acetylmethylamino.

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicylic aromatic rings. For example, bicyclic aromatic rings containing heteroatoms in a hydrocarbon ring structure are referred to as bicyclic heteroaryl rings.

The term “aryl,” as used herein, means a phenyl group, a naphthyl group, an indenyl group or a naphthalenyl group. The aryl groups of the present invention can be optionally substituted with one, two, three, four, or five substituents independently selected from, for example, alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “arylene,” is art-recognized, and as used herein, pertains to a bidentate moiety obtained by removing two hydrogen atoms of an aryl ring, as defined above.

The term “arylalkyl” or “aralkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkoxy” or “arylalkyloxy” as used herein, means an arylalkyl group, as defined herein, appended to the parent molecular moiety through an oxygen. The term “heteroarylalkoxy” as used herein, means an heteroarylalkyl group, as defined herein, appended to the parent molecular moiety through an oxygen.

The term “arylalkylthio” as used herein, means an arylalkyl group, as defined herein, appended to the parent molecular moiety through an sulfur. The term “heteroarylalkylthio” as used herein, means an heteroarylalkyl group, as defined herein, appended to the parent molecular moiety through an sulfur.

The term “arylalkenyl” as used herein, means an aryl group, as defined herein, appended to the parent molecular moiety through an alkenyl group. A representative example is phenylethylenyl.

The term “arylalkynyl” as used herein, means an aryl group, as defined herein, appended to the parent molecular moiety through an alkynyl group. A representative example is phenylethynyl.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “arylcarbonylalkyl” as used herein, means an arylcarbonyl group, as defined herein, bound to the parent molecule through an alkyl group, as defined herein.

The term “arylcarbonylalkoxy” as used herein, means an arylcarbonylalkyl group, as defined herein, bound to the parent molecule through an oxygen.

The term “aryloxy” as used herein, means an aryl group, as defined herein, appended to the parent molecular moiety through an oxygen. The term “heteroaryloxy” as used herein, means a heteroaryl group, as defined herein, appended to the parent molecular moiety through an oxygen.

The term “carbonyl” as used herein, means a —C(═O)— group.

The term “carboxy” as used herein, means a —CO₂H group.

The term “cycloalkyl” as used herein, means monocyclic or multicyclic (e.g., bicyclic, tricyclic, etc.) hydrocarbons containing from 3 to 12 carbon atoms that is completely saturated or has one or more unsaturated bonds but does not amount to an aromatic group. Examples of a cycloalkyl group include cyclopropyl, cyclobutyl, cyclopentyl, cyclopentenyl, cyclohexyl and cyclohexenyl.

The term “cycloalkoxy” as used herein, means a cycloalkyl group, as defined herein, appended to the parent molecular moiety through an oxygen.

The term “cyano” as used herein, means a —CN group.

The term “formyl” as used herein, means a —C(═O)H group.

The term “halo” or “halogen” means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocyclyl”, as used herein, include non-aromatic, ring systems, including, but not limited to, monocyclic, bicyclic and tricyclic rings, which can be completely saturated or which can contain one or more units of unsaturation, for the avoidance of doubt, the degree of unsaturation does not result in an aromatic ring system) and have 3 to 12 atoms including at least one heteroatom, such as nitrogen, oxygen, or sulfur. For purposes of exemplification, which should not be construed as limiting the scope of this invention, the following are examples of heterocyclic rings: azepinyl, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl. The heterocyclyl groups of the invention are substituted with 0, 1, 2, or 3 substituents independently selected, for example, from alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “heteroaryl” as used herein, include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindolyl, benzo[b]thienyl, benzimidazolyl, benzofuranyl, benzoxazolyl, benzothiazolyl, benzothiadiazolyl, benzotriazolyl, benzoxadiazolyl, chromenyl, cinnolinyl, furanyl, furazanyl, imidazolyl, imidazopyridinyl, imidazo[2,1-b]thiazolyl, imidazo[1,2-a]pyridinyl, indenyl, indolizinyl, indolyl, indolinyl, indazolyl, isoindolinyl, isoxazolyl, isothiazolyl, isoquinolinyl, naphthyridinyl, oxadiazolyl, oxazolyl, phthalazinyl, pteridinyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, quinoxalinyl, triazolyl, [1,2,4]triazolo[1,5-a]pyrimidinyl, thiazolyl, thioindolyl, thiophenyl, tetrahydroindolyl, tetrazolyl, thiadiazolyl, thienyl, thiomorpholinyl, triazolyl or tropanyl. The heteroaryl groups of the invention are substituted with 0, 1, 2, or 3 substituents independently selected from, for example, alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “heteroarylene,” is art-recognized, and as used herein, pertains to a bidentate moiety obtained by removing two hydrogen atoms of a heteroaryl ring, as defined above.

The term “heteroarylalkyl” or “heteroaralkyl” as used herein, means a heteroaryl, as defined herein, appended to the parent molecular moiety through an alkyl group, as defined herein. Representative examples of heteroarylalkyl include, but are not limited to, pyridin-3-ylmethyl and 2-(thien-2-yl)ethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “silyl” as used herein includes hydrocarbyl derivatives of the silyl (H₃Si—) group (i.e., (hydrocarbyl)₃Si—), wherein a hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy” as used herein means a silyl group, as defined herein, is appended to the parent molecule through an oxygen atom.

### Pharmaceutical Compositions

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds can be formulated for parenteral administration by injection, e.g., bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, .beta.-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

Suitable bases for forming pharmaceutically acceptable salts with acidic functional groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

### Dosage

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition). In some cases it is appropriate to determine the IC₅₀ in the presence of 3 to 5% serum albumin since such a determination approximates the binding effects of plasma protein on the compound. Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### Exemplary Formulations

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the following Examples.

Capsules containing an active compound can be prepared. In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

Tablets can be prepared, for example, from the ingredients shown in Table 1 below.

TABLE 1

| | Parts by weight |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tabletting machine to give tablets each containing a unit dose or a part of a unit dose of active compound.

The tablets can be enteric coated in a conventional manner using a solution of 20% cellulose acetate phthalate and 3% diethyl phthalate in ethanol:DCM (1:1).

Suppositories containing an active compound can be prepared. In the preparation of suppositories, for example, 100 parts by weight of active compound can be incorporated in 1300 parts by weight of triglyceride suppository base and the mixture formed into suppositories each containing a therapeutically effective amount of active ingredient.

### General Synthetic Schemes

Compounds of the invention may be prepared using the synthetic transformations illustrated in Schemes I-X. Starting materials are commercially available, may be prepared by the procedures described herein, by literature procedures, or by procedures that would be well known to one skilled in the art of organic chemistry.

### PREPARATIONS AND EXAMPLES

The general synthetic methods used in each General Procedure follow and include an illustration of a compound that was synthesized using the designated General Procedure. Compounds of the present invention were synthesized and their activity assayed as described below. Unless otherwise stated, reagents were purchased from Sigma Aldrich, Acros, Alfa Aesar or the Sigma Aldrich Custom Packaged Reagent service. Reagent/reactant names given are as named on the commercial bottle or as generated by IUPAC conventions, CambridgeSoft® ChemDraw Ultra 9.0.7, or AutoNom 2000. Compounds are names as generated by IUPAC conventions, CambridgeSoft® ChemDraw Ultra 9.0.7, or AutoNom 2000.

Importantly, none of the specific conditions and reagents noted in the following are to be construed as limiting the scope of the invention and are provided for illustrative purposes only.

Abbreviations which have been used in the descriptions of the schemes and the examples that follow are listed in Table 1 below:

TABLE 1
List of Abbreviations

| | |
|---|---|
| APCI | Atmospheric pressure chemical ionization |
| BSA | Bovine serum albumin |
| CuCN | Copper cyanide |
| DAD | Diode array |
| DCM | Dichloromethane |
| DIAD | Diisopropyl azodicarboxylate |
| DAD | Diode array |
| DIAD | Diisopropyl azodicarboxylate |
| DIBAH | Diisobutylaluminum hydride |
| DMA | Dimethylacetamide |
| DMSO | Dimethyl sulfoxide |
| EIC | Extracted ion chromatogram |
| ELSD | Evaporative light scattering detector |
| eq | Equivalent |
| Et₂O | Diethyl ether |
| EtOAc | Ethyl acetate |
| GDP | Guanosine-5′-diphosphate |
| h | Hour(s) |
| H₂SO₄ | Sulfuric acid |
| HCl | Hydrochloric acid |
| HEPES | 4-(2-hydroxyeethyl)-1-piperazineethansulfonic acid |
| HOAc | Acetic acid |
| HPLC | High performance liquid chromatography |
| IBX | 2-Iodoxybenzoic acid |
| MgSO₄ | Magnesium sulfate |
| MeOH | Methanol |
| min | Minute(s) |
| MP-NaCNBH₃ | Sodium cyanoborohydride on solid support |
| NaCN | Sodium cyanide |
| NaHCO₃ | Sodium hydrogen carbonate |
| NaHSO₃ | Sodium hydrogen sulfate |
| Na₂SO₄ | Sodium sulfate |
| NMR | Nuclear magnetic resonance |
| PE | Petroleum ether |
| PPh₃ | Triphenyl phosphine |
| RP | Reverse phase |
| Rₜ | Retention time |
| TLC | Thin layer chromatography |

### Analytical Methods

Analytical data is included within the procedures below, in the illustrations of the general procedures, or in the tables of examples. Unless otherwise stated, all ¹H and ¹³C NMR data were collected on a Varian Mercury Plus 400 MHz or a Bruker AVIII 300 MHz instrument; chemical shifts are quoted in parts per million (ppm). HPLC analytical data are either detailed within the experimental or referenced to the table of LC/MS and HPLC conditions, using the lower case letter in Table 2.

TABLE 2
List of LC/MS and GC/MS Methods

| | Unless indicated otherwise mobile phase A was 10 mM ammonium acetate, mobile |
|---|---|
| Method | phase B was HPLC grade acetonitrile |
| a | Analytical LC/MS was performed on a Waters ZMD mass spectrometer and Alliance |
| | HPLC system running MassLynx 3.4 and Openlynx 3.4 software. The ZMD mass |
| | spectrometer was operated under positive APCI ionization conditions. The HPLC |
| | system comprised a Waters 2795 autosampler sampling from 96-well plates, a |
| | Waters 996 diode-array detector and Sedere Sedex-75 evaporative light scattering |
| | detector. The column used was a Phenomenex Luna Combi-HTS C8(2) 5 μm 100 Å |
| | (2.1 mm × 30 mm). A gradient of 10-100% acetonitrile (A) and 0.1% trifluoroacetic |
| | acid in water (B) was used, at a flow rate of 2.0 mL/min (0-0.1 min 10% A, 0.1-2.6 |
| | min 10-100% A, 2.6-2.9 min 100% A, 2.9-3.0 min 100-10% A. 0.5 min post-run |
| | delay. |
| b | Analytical LC/MS was performed on either a Waters LCT Premier mass |
| | spectrometer and 1525 Binary HPLC Pump running MassLynx 4.0 and OpenLynx |
| | 4.0 software or a Waters Quattro Ultima mass spectrometer and Agilent 1100 HPLC |
| | system controlled by MassLynx 4.0 and OpenLynx 4.0 software. The gradient was |
| | 5-60% B in 1.5 min then 60-95% B to 2.5 min with a hold at 95% B for 1.2 min (1.3 |
| | mL/min flow rate). Mobile phase A was 10 mM ammonium acetate, mobile phase B |
| | was HPLC grade acetonitrile. The column used for the chromatography was a |
| | 4.6 × 50 mm MAC-MOD Halo C8 column (2.7 μm particles). Detection methods are |
| | DAD and ELSD detection as well as positive/negative electrospray ionization.) |
| c | Analytical LC/MS was performed on an Agilent 1200 HPLC/6100 SQ System. |
| | Mobile Phase: A: Water (0.05% TFA) B: Acetonitirle (0.05% TFA); Gradient |
| | Phase: 5%-95% in 1.3 min; Flow rate: 1.6 mL/min; Column: XBridge, 2.5 min; |
| | Oven Temp. 50° C. |
| d | GC/MS was performed on an Agilent 7890A GC 5975C VL MSD with FID |
| | detector. The column is: Agilent HT-5MS, 30 m*250 μm*0.25 μm. Flow rate: 2 |
| | mL/min using He as carrier gas. The operation procedure was: 60° C. for 2 min, 60 to |
| | 250° C. at a rate of 20° C./min, 250° C. for 3 min. MS is EI. |
| e | Chiral HPLC measurement was performed on a Shimadzu LC-20AD mounted the |
| | Daicel chiral column AS-H 4.6 × 205 mm under the following conditions: Mobile |
| | phase: Ethanol:Hexane (25:75) (0.1% TFA). Temperature: 25° C. Flow rate: 1 min. |
| | Signal was detected on 214 nm and 254 nm detector. |
| f | Analytical LC/MS was performed on a Finnigan Navigator mass spectrometer and |
| | Agilent 1100 HPLC system running Xcalibur 1.2, Open-Access 1.3, and custom |
| | login software. The mass spectrometer was operated under positive APCI ionization |
| | conditions. The HPLC system comprised an Agilent Quaternary pump, degasser, |
| | column compartment, autosampler and diode-array detector, with a Sedere Sedex 75 |
| | evaporative light-scattering detector. The column used was a Phenomenex Luna |
| | Combi-HTS C8(2) 5 μm 100 Å (2.1 mm × 30 mm). The gradient was 10-100% |
| | acetonitrile (A) and 0.1% trifluoroacetic acid in water (B), at a flow rate of 2.0 |
| | mL/min (0-0.1 min 10% A, 0.1-2.6 min 10-100% A, 2.6-2.9 min 100% A, 2.9-3.0 |
| | min 100-10% A. 0.5 min post-run delay). |

### General Procedure A: Synthesis of Compounds in Table A

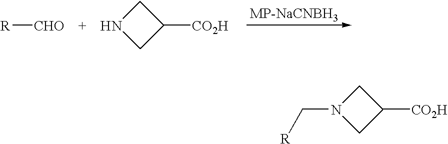

Compounds in Table A were produced as part of a one dimensional array with the only variant being the aldehyde monomer which is given in Table A. The aldehydes were purchased pre-weighed from the Sigma Aldrich Custom Packaged Reagent service.

In a 20 mL vial a solution of an aldehyde monomer (1.2 eq) dissolved in DCM (1.2 mL) was added, followed by the addition of an azetidine-3-carboxylic acid (25 mg, 1 eq) dissolved in DCM (1.0 mL), followed by HOAc (5 eq) dissolved in DCM (0.3 mL), followed by MP-cyanoborohydride resin (Biotage, 2 eq). The mixture was shaken at room temperature for about 5 h. The reaction was checked by LC/MS and concentrated to dryness. The residue was dissolved in 1:1 DMSO:MeOH and purified by preparative HPLC on a Phenomenex Luna C8(2) 5 μm 100 Å AXIA column (30 mm×75 mm) A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE A

| | | | LC/MS | | M + 1 |
|---|---|---|---|---|---|
| | | | Rₜ | Observed | or |
| Ex. # | Starting aldehyde | Structure | (min) | mass | M − 1 |
| A.1 | 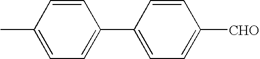 | 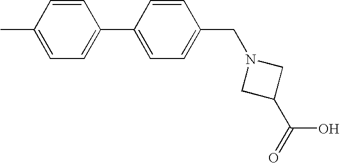 | 1.35 | 282 | M + 1 |
| A.2 | 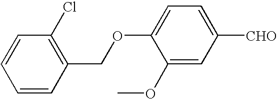 | 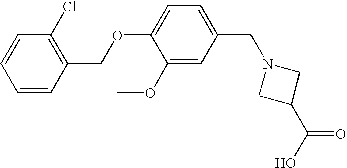 | 1.27 | 360 | M − 1 |
| A.3 | 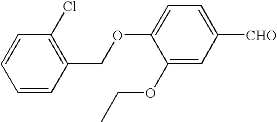 | 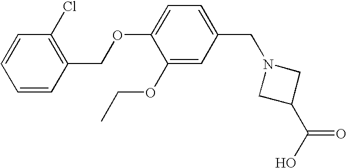 | 1.39 | 374 | M − 1 |
| A.4 | 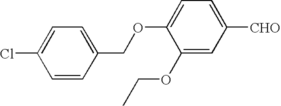 | 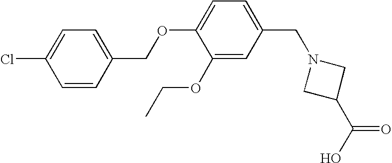 | 1.27 | 374 | M − 1 |
| A.5 | 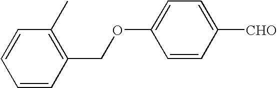 | 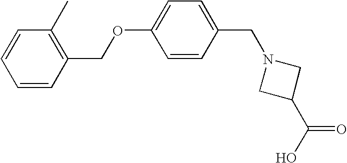 | 1.3 | 310 | M − 1 |
| A.6 | 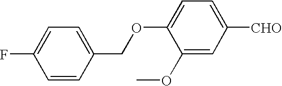 | 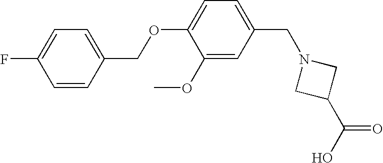 | 1.21 | 344 | M − 1 |
| A.7 | 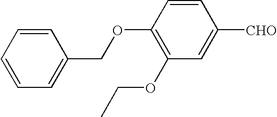 | 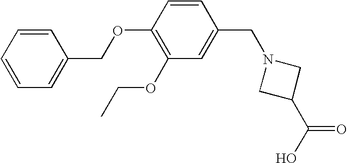 | 1.27 | 340 | M − 1 |
| A.8 | 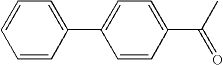 | 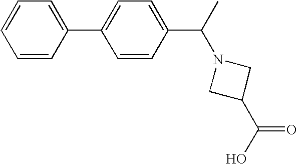 | 1.26 | 280 | M − 1 |
| A.9 | 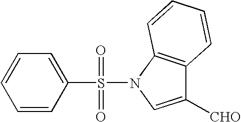 | 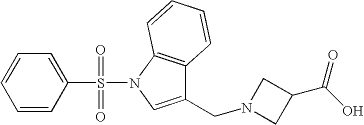 | 1.27 | 369 | M − 1 |
| A.10 | 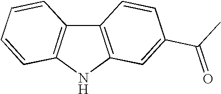 | 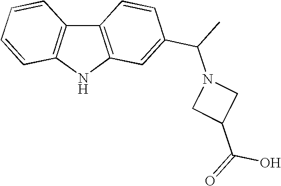 | 1.2 | 293 | M − 1 |
| A.11 | 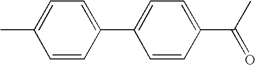 | 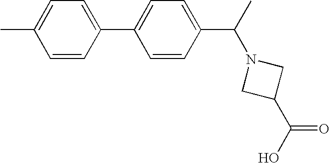 | 1.36 | 294 | M − 1 |
| A.12 | 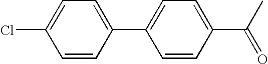 | 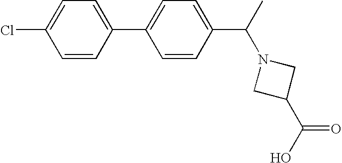 | 1.38 | 314 | M − 1 |
| A.13 | 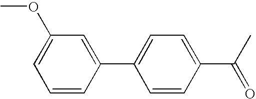 | 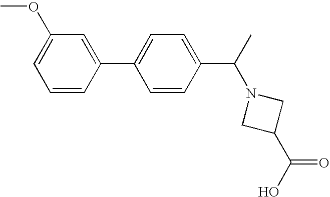 | 1.26 | 310 | M − 1 |
| A.14 | 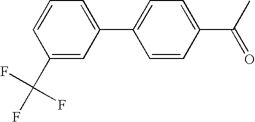 | 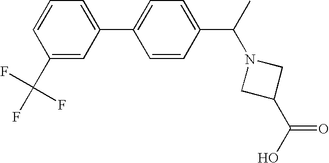 | 1.39 | 348 | M − 1 |
| A.15 | 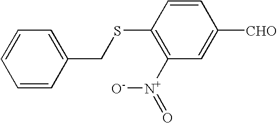 | 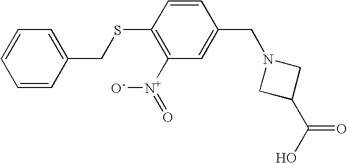 | 1.38 | 357 | M − 1 |

### General Procedure B: Synthesis of Compounds in Table B

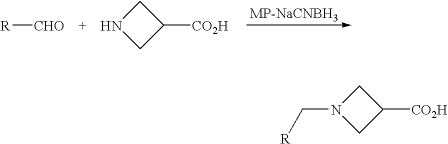

Compounds in Table B were produced as part of a one dimensional array with the only variant being the aldehyde monomer which is given in Table B. The aldehydes were purchased pre-weighed from the Sigma Aldrich Custom Packaged Reagent service.

In a 20 mL vial a solution of an aldehyde monomer (1.2 eq) dissolved in DCM (1.2 mL) was added, followed by the addition of azetidine-3-carboxylic acid (27 mg, 1 eq) dissolved in DCM (1.0 mL), followed by HOAc (5 eq) dissolved in DCM (0.3 mL), followed by MP-cyanoborohydride resin (Biotage, 2 eq). The mixture was shaken at room temperature for about 5 h. The reaction was checked by LC/MS and concentrated to dryness. The residue was dissolved in 1:1 DMSO:MeOH and purified by preparative HPLC on a Phenomenex Luna C8(2) 5 μm 100 Å AXIA column (30 mm×75 mm). A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE B

| | | | LC/MS | | M + 1 |
|---|---|---|---|---|---|
| | | | Rₜ | Observed | or |
| Ex. # | Starting aldehyde | Structure | (min) | mass | M − 1 |
| B.1 | 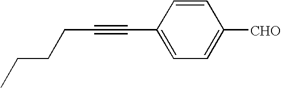 | 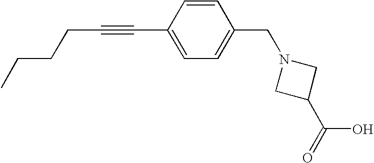 | 1.37 | 272 | M + 1 |
| B.2 | 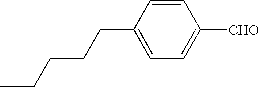 | 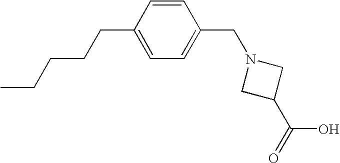 | 1.35 | 262 | M + 1 |
| B.3 | 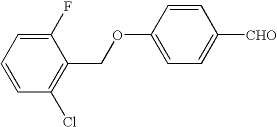 | 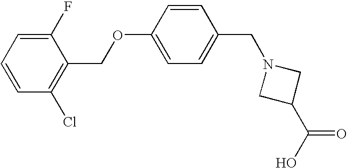 | 1.29 | 348 | M − 1 |
| B.4 | 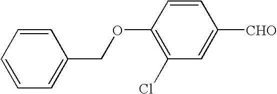 | 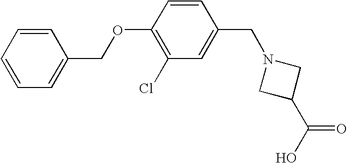 | 1.18 | 330 | M − 1 |
| B.5 | 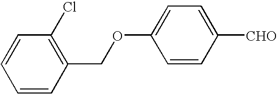 | 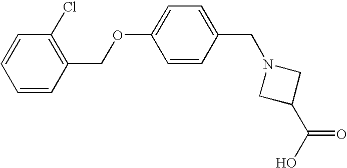 | 1.35 | 329 | M − 1 |
| B.6 | 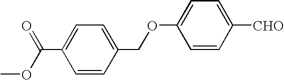 | 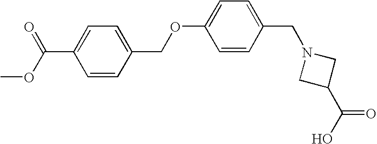 | 1.15 | 354 | M − 1 |
| B.7 | 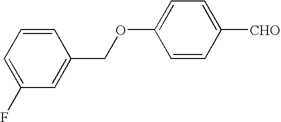 | 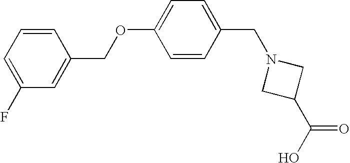 | 1.21 | 314 | M − 1 |
| B.8 | 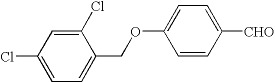 | 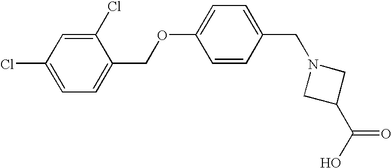 | 1.41 | 364 | M − 1 |
| B.9 | 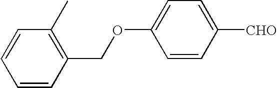 | 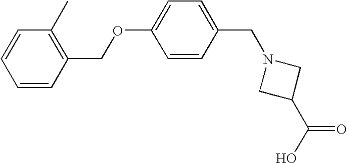 | 1.29 | 310 | M − 1 |
| B.10 | 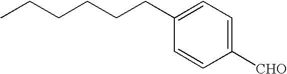 | 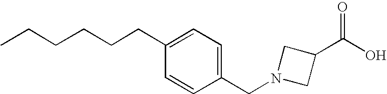 | 1.39 | 274 | M − 1 |
| B.11 | 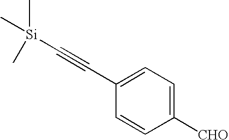 | 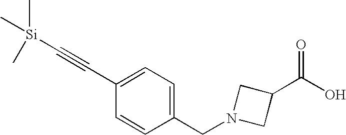 | 1.53 | 288 | M + 1 |

### General Procedure C: Synthesis of Compounds in Table C

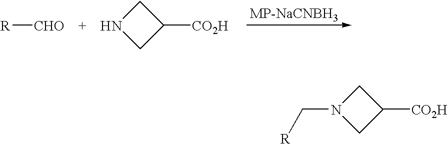

Compounds in Table C were produced as part of a one dimensional array with the only variant being the aldehyde monomer which is given in Table C. The aldehydes were purchased pre-weighed from the Sigma Aldrich Custom Packaged Reagent service.

A 20 mL vial was charged with a solution of an aldehyde monomer in DCM (0.6 mmol pre-weighed, 1.20 eq, 2.0 mL DCM), MP-cyanoborohydride resin (Biotage, 3.0 eq), azetidine-3-carboxylic acid in 1.0 mL of DCM (1.0 eq, 41.67 mg), and a solution of HOAc in DCM (3.0 eq, 59.34 mmol total, 76.24 μL, 500 μL of DCM). This was capped and shaken for about 4-5 h, monitoring the reaction until there was complete product formation. After product formation the material was filtered and the solvent was removed in vacuo (Speed Vac) from the crude mixture. The material was then dissolved in 1.4 mL of DMSO:MeOH solution (1:1 v/v) and purified by preparative HPLC on a Phenomenex Luna C8(2) 5 um 100 Å AXIA column (30 mm×75 mm). A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE C

| | | | LC/MS | | M + 1 |
|---|---|---|---|---|---|
| | | | Rₜ | Observed | or |
| Ex. # | Starting aldehyde | Structure | (min) | mass | M − 1 |
| C.1 | 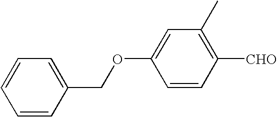 | 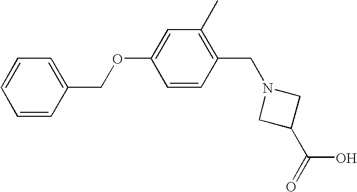 | 1.16 | 312.4 | M + 1 |
| C.2 | 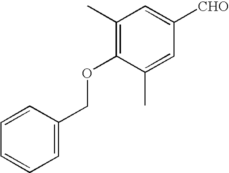 | 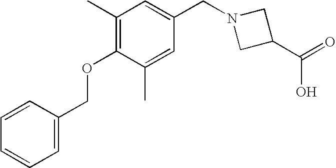 | 1.2 | 326.4 | M + 1 |
| C.3 | 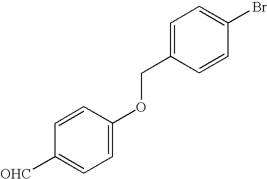 | 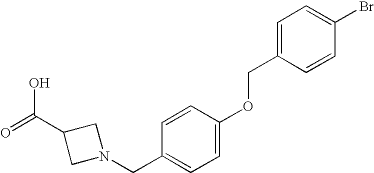 | 1.25 | 376.3 | M + 1 |
| C.4 | 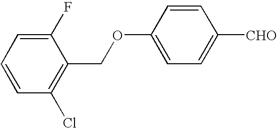 | 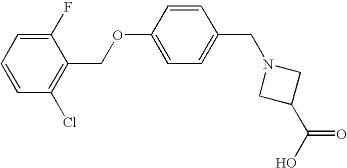 | 1.18 | 350.3 | M + 1 |
| C.5 | 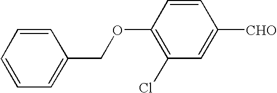 | 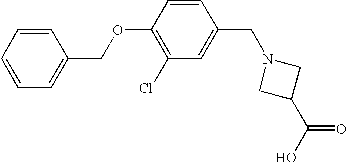 | 1.21 | 332.3 | M + 1 |
| C.6 | 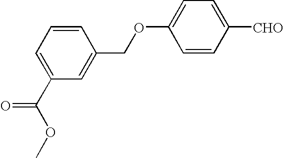 | 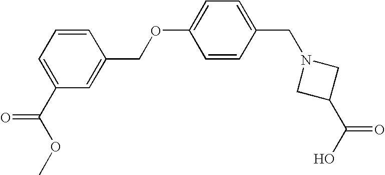 | 1.12 | 356.4 | M + 1 |
| C.7 | 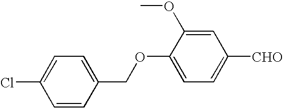 | 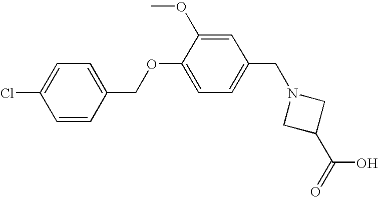 | 1.2 | 362.3 | M + 1 |
| C.8 | 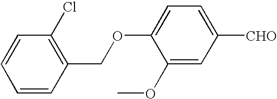 | 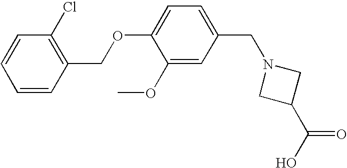 | 1.17 | 362.3 | M + 1 |
| C.9 | 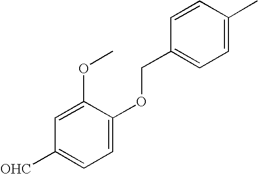 | 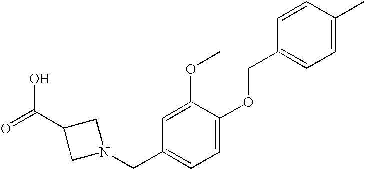 | 1.18 | 342.4 | M + 1 |
| C.10 | 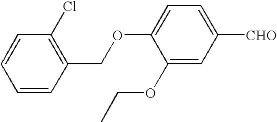 | 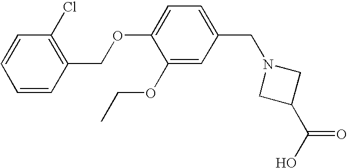 | 1.25 | 376.4 | M + 1 |
| C.11 | 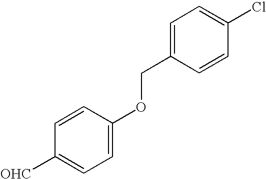 | 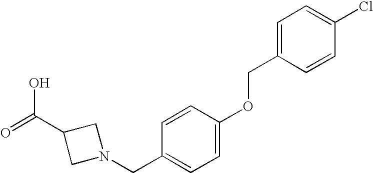 | 1.225 | 332.3 | M + 1 |
| C.12 | 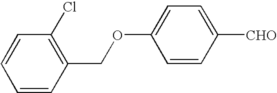 | 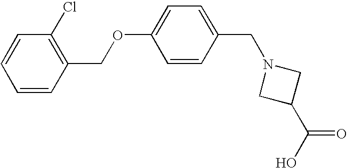 | 1.23 | 332.3 | M + 1 |
| C.13 | 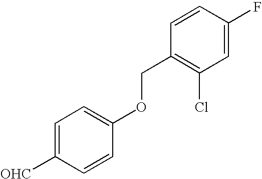 | 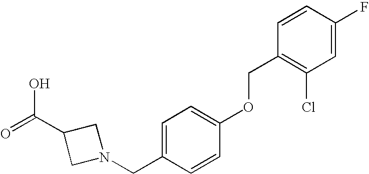 | 1.22 | 350.3 | M + 1 |
| C.14 | 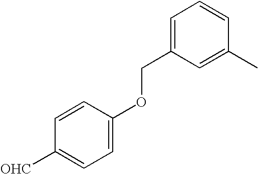 | 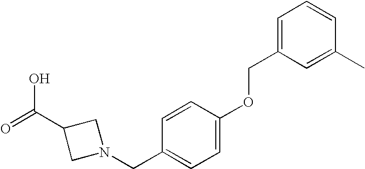 | 1.2 | 312.4 | M + 1 |
| C.15 | 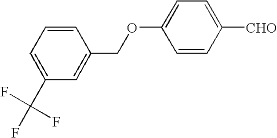 | 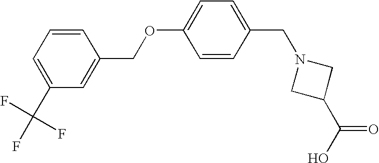 | 1.28 | 366.3 | M + 1 |
| C.16 | 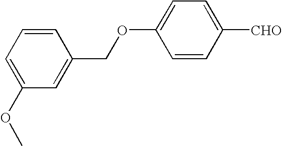 | 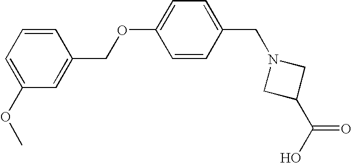 | 1.12 | 328.4 | M + 1 |
| C.17 | 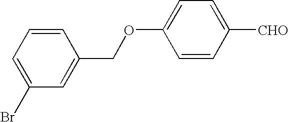 | 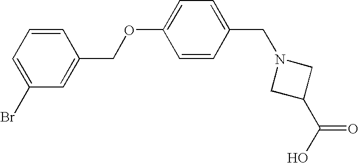 | 1.23 | 376.3 | M + 1 |
| C.18 | 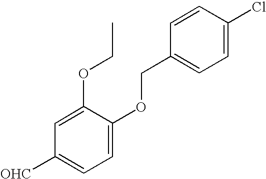 | 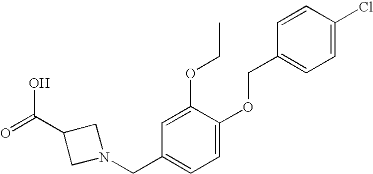 | 1.26 | 376.3 | M + 1 |
| C.19 | 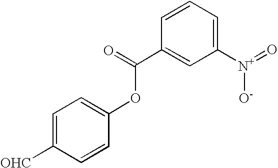 | 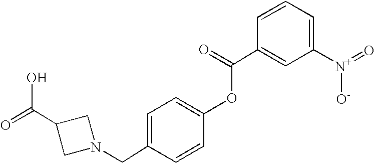 | 1.08 | 357.3 | M + 1 |
| C.20 | 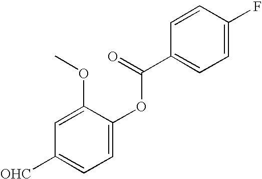 | 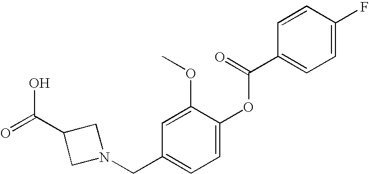 | 1.15 | 360.3 | M + 1 |
| C.21 | 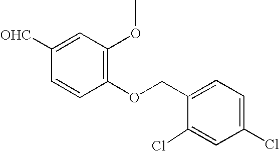 | 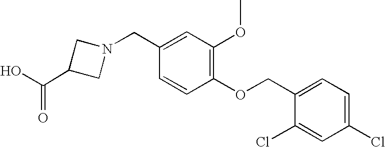 | 1.27 | 396.3 | M + 1 |
| C.22 | 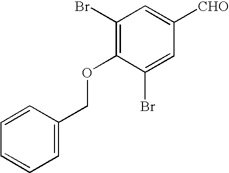 | 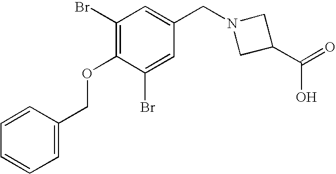 | 1.26 | 454.2 | M + 1 |
| C.23 | 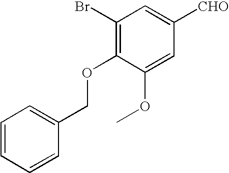 | 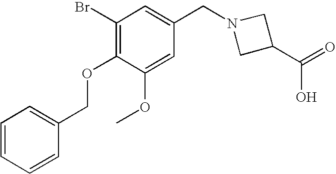 | 1.23 | 406.3 | M + 1 |
| C.24 | 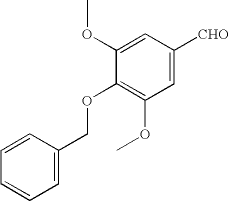 | 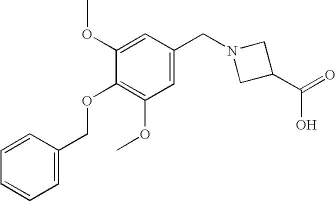 | 1.12 | 358.4 | M + 1 |
| C.25 | 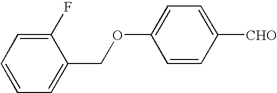 | 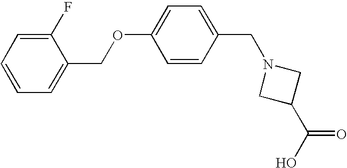 | 1.12 | 316.4 | M + 1 |
| C.26 | 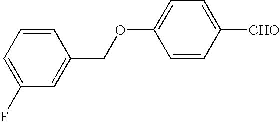 | 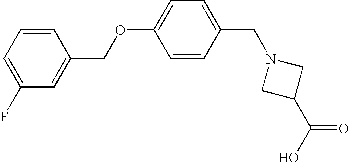 | 1.14 | 316.4 | M + 1 |
| C.27 | 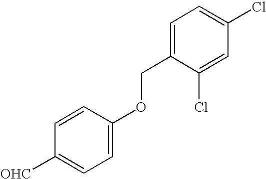 | 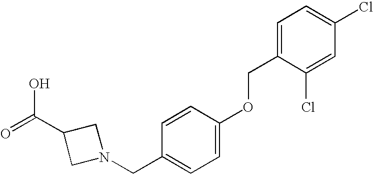 | 1.14 | 366.3 | M + 1 |
| C.28 | 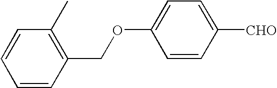 | 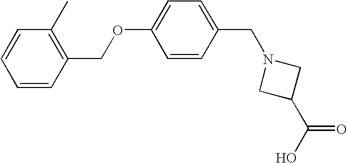 | 1.14 | 312.4 | M + 1 |
| C.29 | 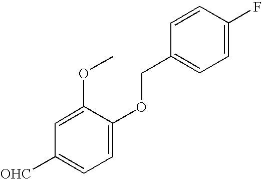 | 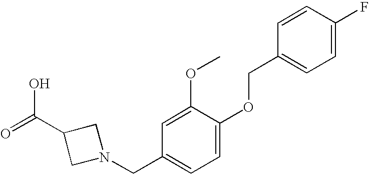 | 1.12 | 346.4 | M + 1 |
| C.30 | 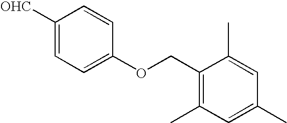 | 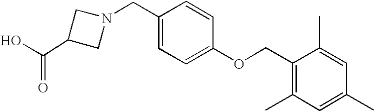 | 1.32 | 340.4 | M + 1 |
| C.31 | 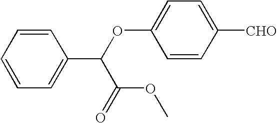 | 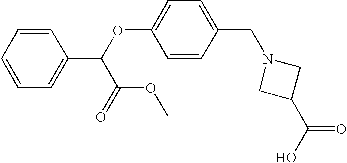 | 1.07 | 356.4 | M + 1 |
| C.32 | 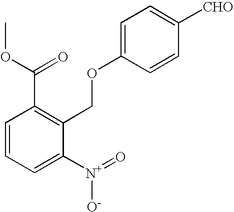 | 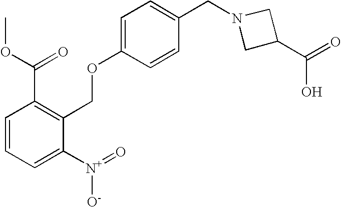 | 1.1 | 401.4 | M + 1 |
| C.33 | 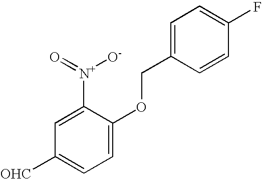 | 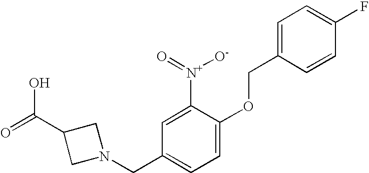 | 1.18 | 361.3 | M + 1 |
| C.34 | 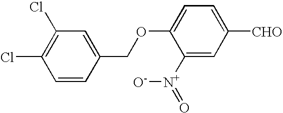 | 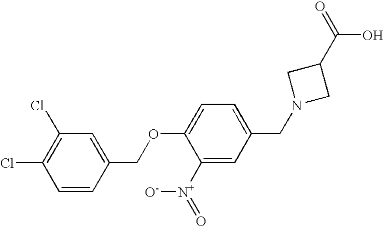 | 1.35 | 411.3 | M + 1 |
| C.35 | 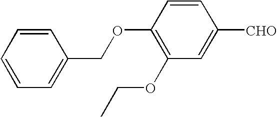 | 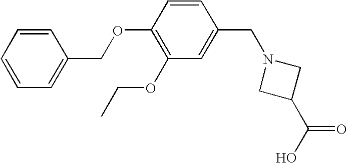 | 1.15 | 342.4 | M + 1 |
| C.36 | 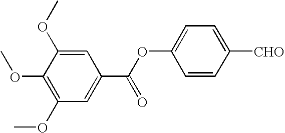 | 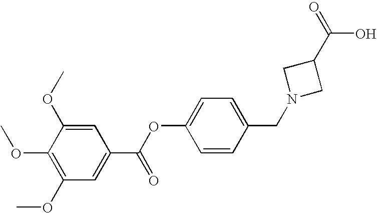 | 1.07 | 402.4 | M + 1 |
| C.37 | 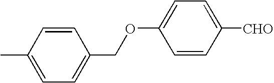 | 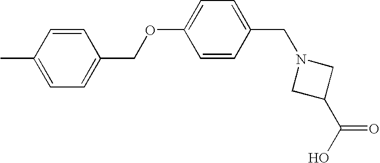 | 1.2 | 312.4 | M + 1 |
| C.38 | 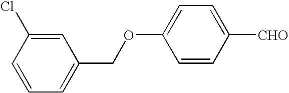 | 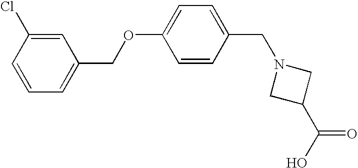 | 1.215 | 332.3 | M + 1 |

### General Procedure D: Synthesis of Compounds in Table D

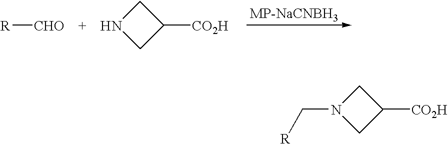

Compounds in Table D were produced as part of a one dimensional array with the only variant being the aldehyde monomer which is given in Table D. The aldehydes were purchased pre-weighed from the Sigma Aldrich Custom Packaged Reagent service.

A 20 mL vial was charged with a solution of an aldehyde monomer in DCM (0.6 mmol pre-weighed, 1.20 eq, 1.0 DCM), MP-cyanoborohydride resin (Biotage, 3.0 eq.), azetidine-3-carboxylic acid in 1.0 mL of DCM (1.0 eq, 45.45 mg), and a solution of HOAc in DCM (3.0 eq, 59.34 mmol total, 81.27 μL, 864 μL of DCM). This was capped and shaken for about 4-5 h, monitoring the reaction until there was complete product formation. After product formation the material was filtered and the solvent was removed in vacuo (Speed Vac) from the crude mixture. The material was then dissolved in 1.4 mL of DMSO:MeOH solution (1:1 v/v) and purified by preparative HPLC on a Phenomenex Luna C8(2) 5 μm 100 Å AXIA column (30 mm×75 mm). A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE D

| | | | LC/MS | | M + 1 |
|---|---|---|---|---|---|
| | | | Rₜ | Observed | or |
| Ex. # | Starting aldehyde | Structure | (min) | mass | M − 1 |
| D.1 | 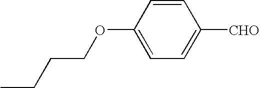 | 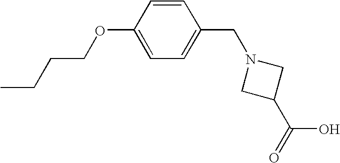 | 1.25 | 264.1 | M + 1 |
| D.2 | 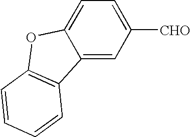 | 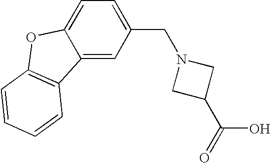 | 1.26 | 282 | M + 1 |
| D.3 | 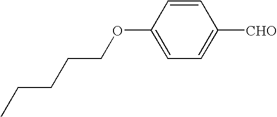 | 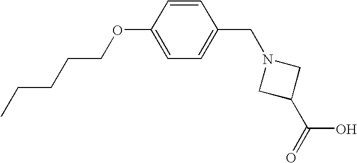 | 1.36 | 278.1 | M + 1 |
| D.4 | 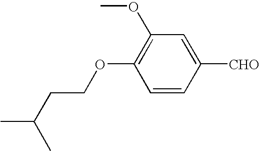 | 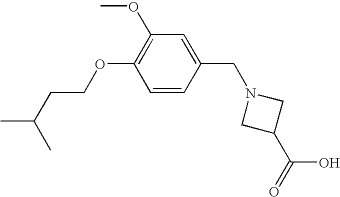 | 1.3 | 308.1 | M + 1 |
| D.5 | 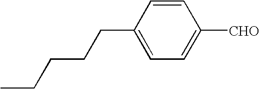 | 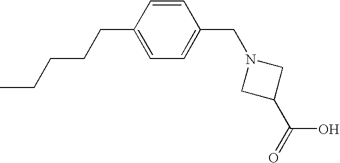 | 1.41 | 262.1 | M + 1 |
| D.6 | 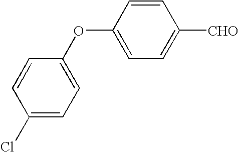 | 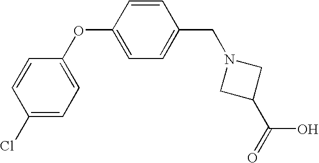 | 1.39 | 318 | M + 1 |
| D.7 | 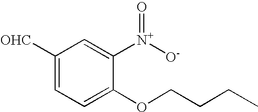 | 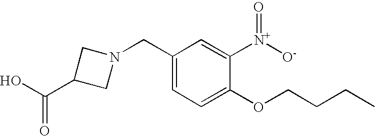 | 1.28 | 309.1 | M + 1 |
| D.8 | 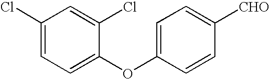 | 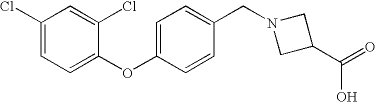 | 1.46 | 351.9 | M + 1 |
| D.9 | 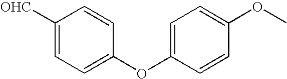 | 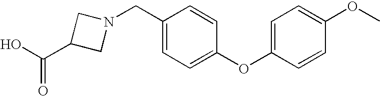 | 1.28 | 314 | M + 1 |
| D.10 | 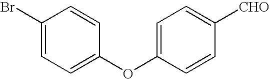 | 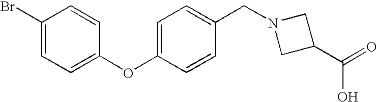 | 1.41 | 361.9 | M + 1 |
| D.11 | 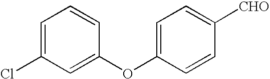 | 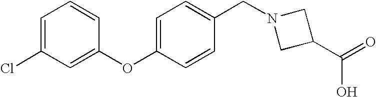 | 1.38 | 318 | M + 1 |
| D.12 | 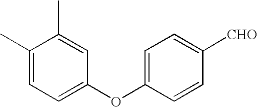 | 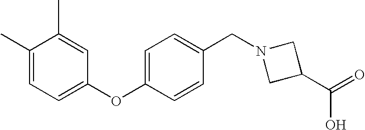 | 1.4 | 312.1 | M + 1 |
| D.13 | 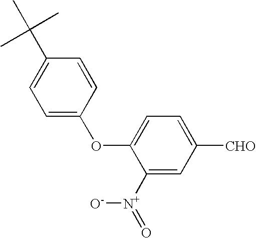 | 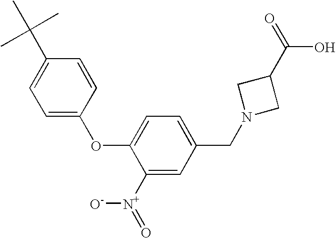 | 1.56 | 385.1 | M + 1 |
| D.14 | 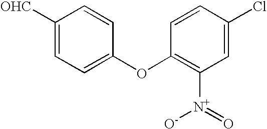 | 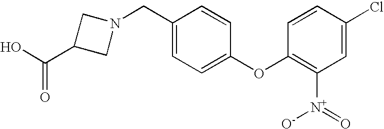 | 1.36 | 363 | M + 1 |
| D.15 | 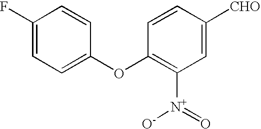 | 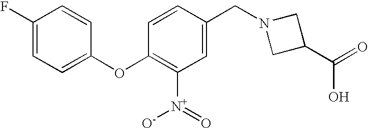 | 1.3 | 347 | M + 1 |
| D.16 | 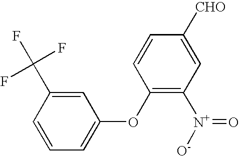 | 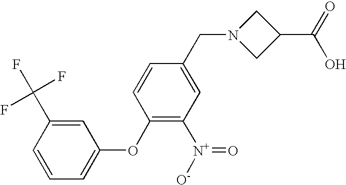 | 1.45 | 396.9 | M + 1 |
| D.17 | 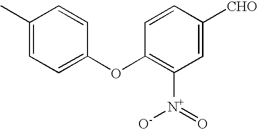 | 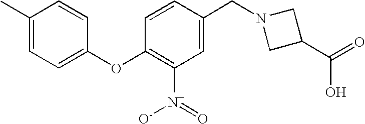 | 1.36 | 343 | M + 1 |
| D.18 | 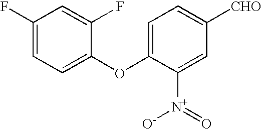 | 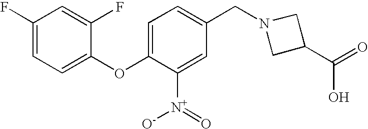 | 1.34 | 365 | M + 1 |
| D.19 | 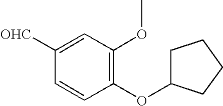 | 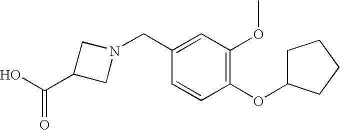 | 1.18 | 306.1 | M + 1 |
| D.20 | 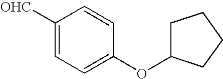 | 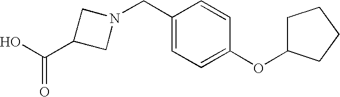 | 1.2865 | 276.1 | M + 1 |
| D.21 | 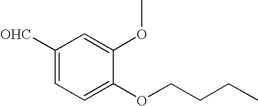 | 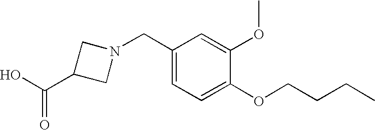 | 1.22 | 294.1 | M + 1 |

### General Procedure E: Synthesis of Compounds in Table E

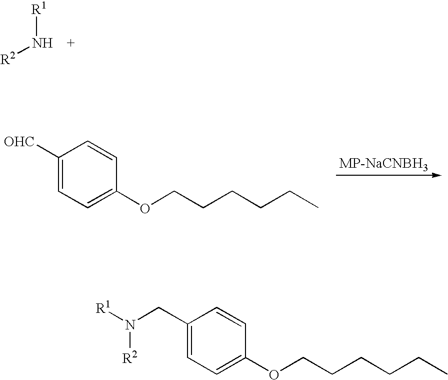

Compounds in Table E were produced as part of a one dimensional array with the only variant being the amine monomer which is given in Table E. The amines were purchased pre-weighed from the Sigma Aldrich Custom Packaged Reagent service.

A 20 mL vial was charged with a solution of 4-(hexyloxy)benzaldehyde in MeOH/DCM (1:1 v/v, 1.0 mL) (20.0 mg, 1 eq, 0.102 mmol), a solution of the amine monomer in DMA (1.20 eq, 0.6 mmol pre-weighed, 2.0 mL DMA), a solution of HOAc in MeOH:DCM (5.0 eq, 0.508 mmol), and MP-cyanoborohydride resin (Biotage, 3 eq.). The vial was capped and placed in a heater shaker at about 55° C. for about 72 h. Once the reaction was complete the resin was removed through filtration and the solvent was removed in vacuo. The crude material was dissolved in 1.4 mL of DMSO:MeOH (1:1 v/v) and purified by preparative HPLC on a Phenomenex Luna C8(2) 5 μm 100 Å AXIA column (30 mm×75 mm). A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE E

| | | | LC/MS | | M + 1 |
|---|---|---|---|---|---|
| | | | Rₜ | Observed | or |
| Ex. # | Starting amine | Structure | (min) | mass | M − 1 |
| E.1 | 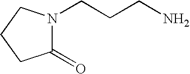 | 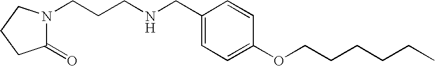 | 1.37 | 333.5 | M + 1 |
| E.2 | 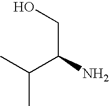 | 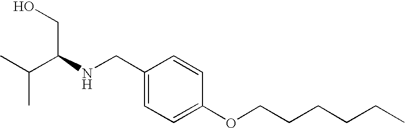 | 1.3 | 294.5 | M + 1 |
| E.3 | 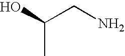 | 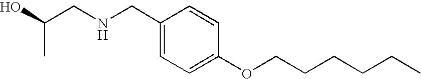 | 1.25 | 266.5 | M + 1 |
| E.4 | 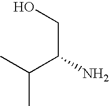 | 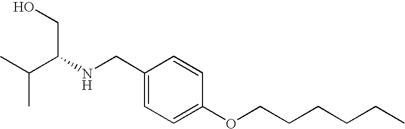 | 1.38 | 294.5 | M + 1 |
| E.5 | 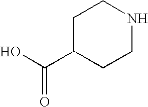 | 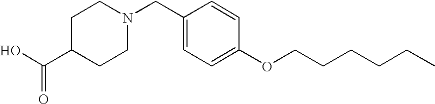 | 1.35 | 320.4 | M + 1 |
| E.6 | 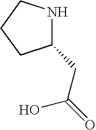 | 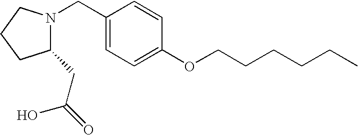 | 1.35 | 320.5 | M + 1 |
| E.7 | 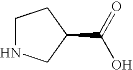 | 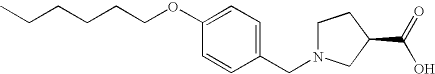 | 1.34 | 306.4 | M + 1 |
| E.8 | 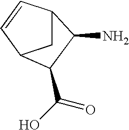 | 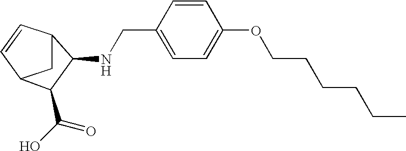 | 1.42 | 344.4 | M + 1 |
| E.9 | 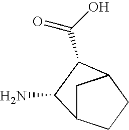 | 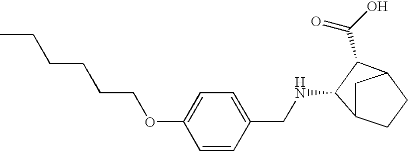 | 1.4 | 346.4 | M + 1 |
| E.10 | 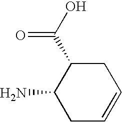 | 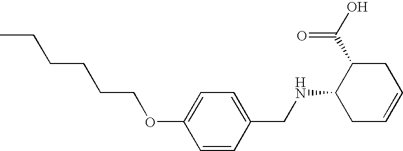 | 1.4 | 332.4 | M + 1 |
| E.11 | 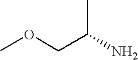 | 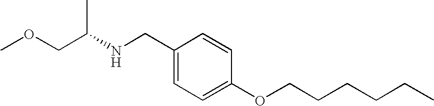 | 1.35 | 280.5 | M + 1 |
| E.12 | 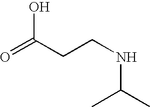 | 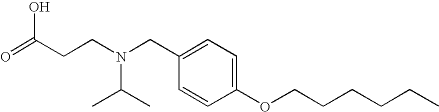 | 1.38 | 322.5 | M + 1 |
| E.13 | 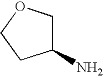 | 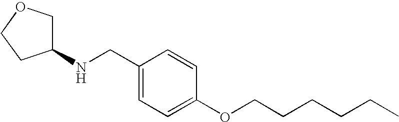 | 1.3 | 278.5 | M + 1 |
| E.14 | 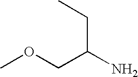 | 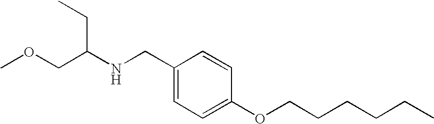 | 1.45 | 294.5 | M + 1 |
| E.15 | 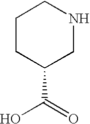 | 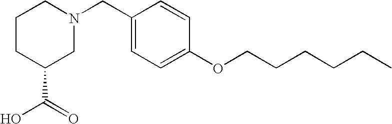 | 1.38 | 320.5 | M + 1 |
| E.16 | 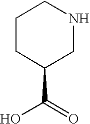 | 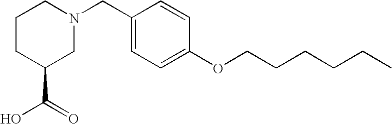 | 1.35 | 320.4 | M + 1 |
| E.17 | 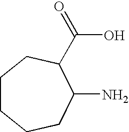 | 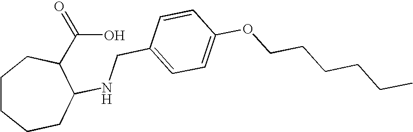 | 1.5 | 348.4 | M + 1 |
| E.18 | 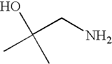 | 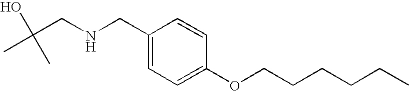 | 1.34 | 280.5 | M + 1 |
| E.19 | 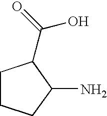 | 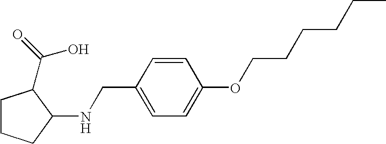 | 1.4 | 320.5 | M + 1 |
| E.20 | 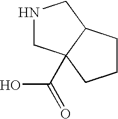 | 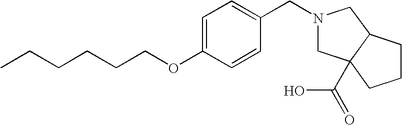 | 1.43 | 346.5 | M + 1 |
| E.21 | 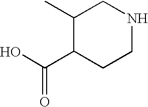 | 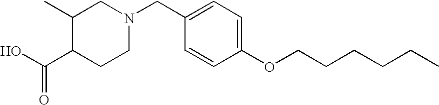 | 1.4 | 334.4 | M + 1 |
| E.22 | 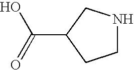 | 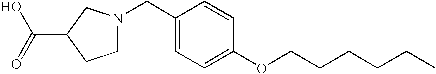 | 1.35 | 306.4 | M + 1 |
| E.23 | 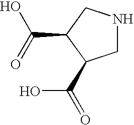 | 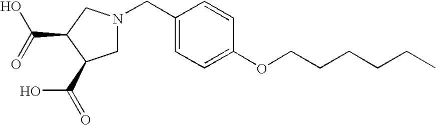 | 1.33 | 350.4 | M + 1 |

### General Procedure F: Synthesis of Compounds in Table F

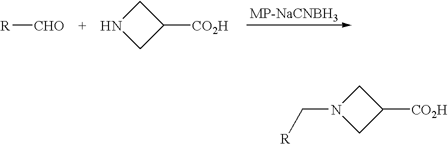

Compounds in Table F were produced as part of a one dimensional array with the only variant being the aldehyde monomer which is given in Table F. The aldehydes were purchased pre-weighed from the Sigma Aldrich Custom Packaged Reagent service.

In a 20 mL vial a solution of the aldehyde monomer (1.2 eq) dissolved in DCM (1.2 mL) was added, followed by the addition of azetidine-3-carboxylic acid (32 mg, 1 eq) dissolved in DCM (1.0 mL), followed by HOAc (3 eq) dissolved in DCM (0.3 mL), followed by MP-cyanoborohydride resin (Biotage, 3 eq.) The mixture was shaken at RT for about 5 h. The reaction was checked by LC/MS and concentrated to dryness. The residue was dissolved in 1:1 DMSO:MeOH and purified by preparative HPLC on a Phenomenex Luna C8(2) 5 μm 100 Å AXIA column (30 mm×75 mm) A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE F

| | | | LC/ | | |
|---|---|---|---|---|---|
| | | | MS | Ob- | M + 1 |
| | | | Rₜ | served | or |
| Ex. # | Starting aldehyde | Structure | (min) | mass | M − 1 |
| F.1 | 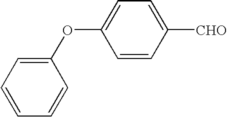 | 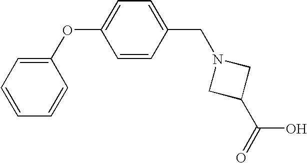 | 1.1 | 284 | M + 1 |
| F.2 | 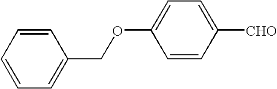 | 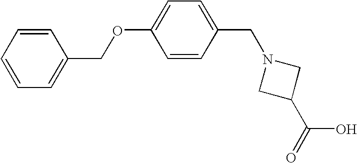 | 1.16 | 298 | M + 1 |
| F.3 | 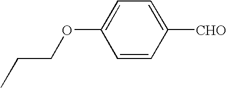 | 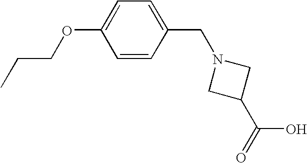 | 0.9 | 250 | M + 1 |
| F.4 | 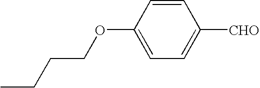 | 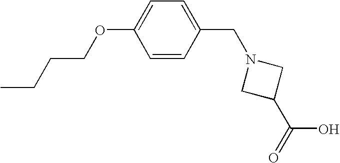 | 1.12 | 264 | M + 1 |
| F.5 | 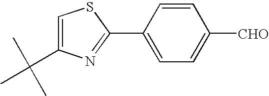 | 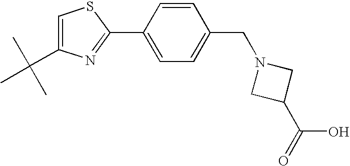 | 1.4 | 331 | M + 1 |
| F.6 | 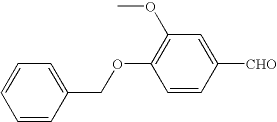 | 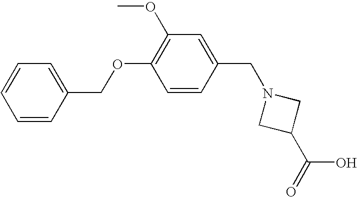 | 1.12 | 328 | M + 1 |
| F.7 | 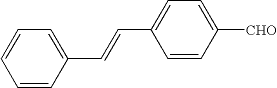 | 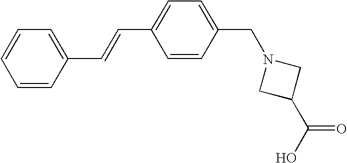 | 1.36 | 294 | M + 1 |
| F.8 | 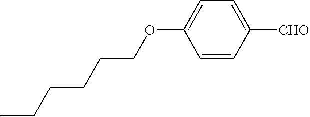 | 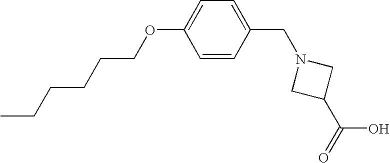 | 1.43 | 292 | M + 1 |
| F.9 | 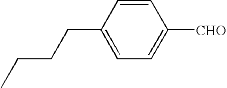 | 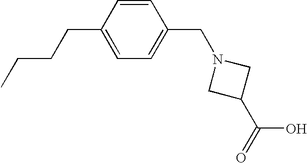 | 1.2 | 248 | M + 1 |
| F.10 | 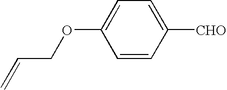 | 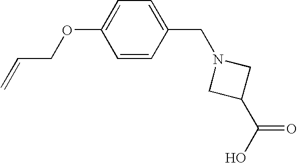 | 0.8 | 248 | M + 1 |
| F.11 | 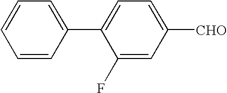 | 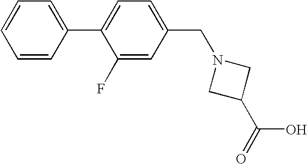 | 1.15 | 286 | M + 1 |
| F.12 | 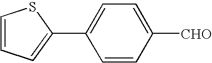 | 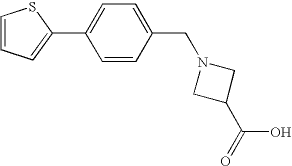 | 1.07 | 274 | M + 1 |
| F.13 | 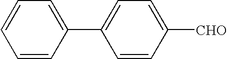 | 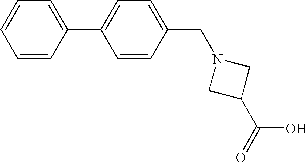 | 1.13 | 268 | M + 1 |
| F.14 | 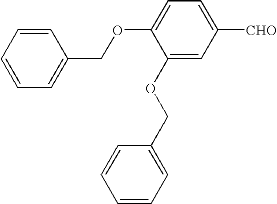 | 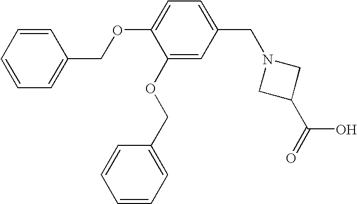 | 1.55 | 404 | M + 1 |
| F.15 | 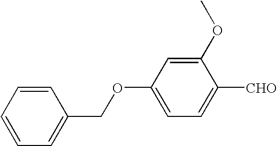 | 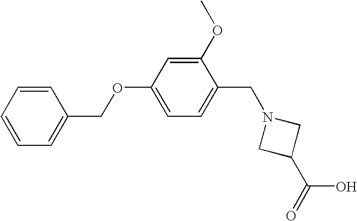 | 1.26 | 328 | M + 1 |
| F.16 | 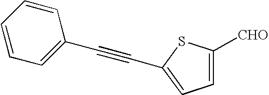 | 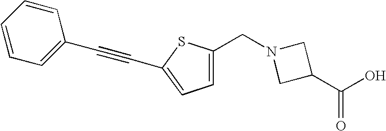 | 1.3 | 296 | M − 1 |
| F.17 | 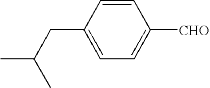 | 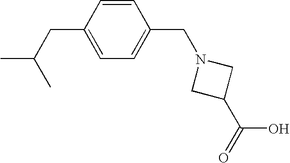 | 1.22 | 248 | M + 1 |
| F.18 | 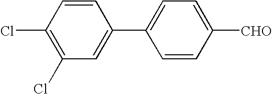 | 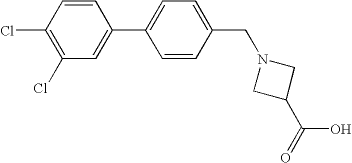 | 1.45 | 336 | M + 1 |
| F.19 | 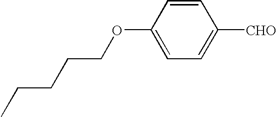 | 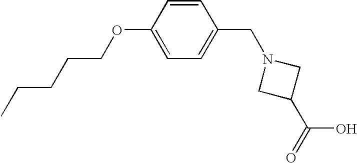 | 1.3 | 278 | M + 1 |

### General Procedure G: Synthesis of Compounds in Table G

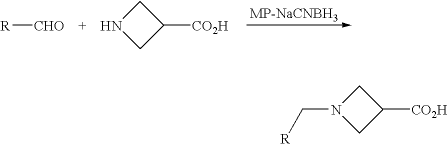

Compounds in Table G were produced as part of a one dimensional array with the only variant being the aldehyde monomer which is given in Table G. The aldehydes were purchased pre-weighed from the Sigma Aldrich Custom Packaged Reagent service.

In a 20 mL vial a solution of the aldehyde monomer (1.2 eq) dissolved in DCM (1.0 mL) was added, followed by the addition of azetidine-3-carboxylic acid (26 mg, 1 eq) dissolved in DCM (1.0 mL), followed by HOAc (3 eq) dissolved in DCM (0.4 mL), followed by MP-cyanoborohydride resin (Biotage, 3 eq). The mixture was shaken at RT for about 5 h. The reaction was checked by LC/MS and concentrated to dryness. The residue was dissolved in 1:1 DMSO:MeOH and purified by preparative HPLC on a Phenomenex Luna C8(2) 5 um 100 Å AXIA column (30 mm×75 mm) A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE G

| | | | LC/ | | |
|---|---|---|---|---|---|
| | | | MS | Ob- | M + 1 |
| | | | Rₜ | served | or |
| Ex. # | Starting aldehyde | Structure | (min) | mass | M − 1 |
| G.1 | 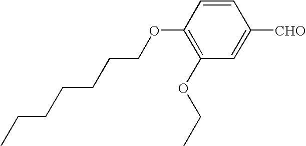 | 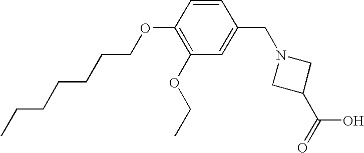 | 1.58 | 350 | M + 1 |
| G.2 | 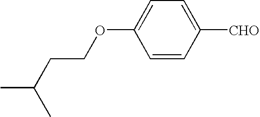 | 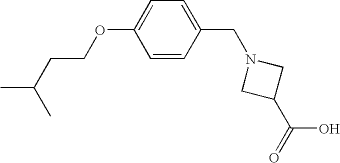 | 1.22 | 278 | M + 1 |
| G.3 | 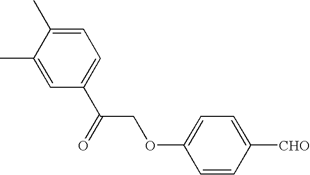 | 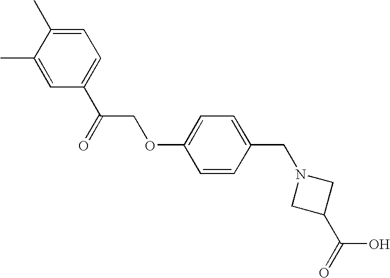 | 1.2 | 354 | M + 1 |
| G.4 | 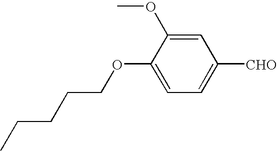 | 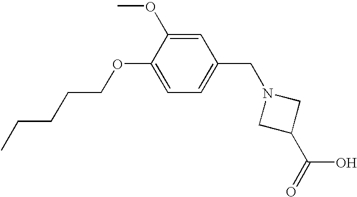 | 1.16 | 308 | M + 1 |
| G.5 | 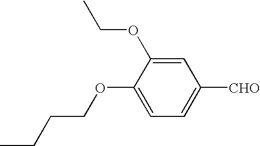 | 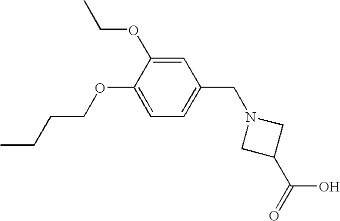 | 1.12 | 308 | M + 1 |
| G.6 | 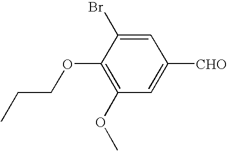 | 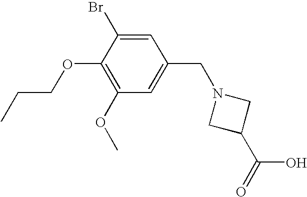 | 1.12 | 358 | M + 1 |
| G.7 | 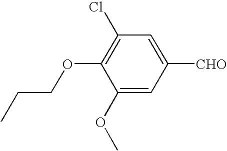 | 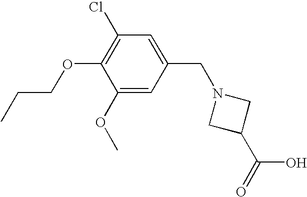 | 1.07 | 314 | M + 1 |
| G.8 | 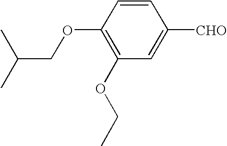 | 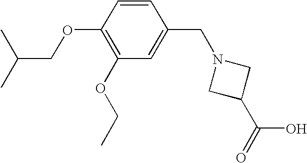 | 1.16 | 306.1 | M − 1 |
| G.9 | 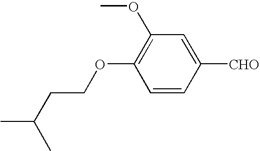 | 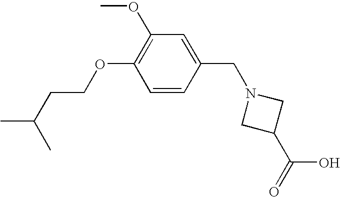 | 1.14 | 308 | M + 1 |
| G.10 | 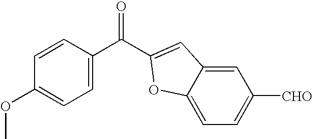 | 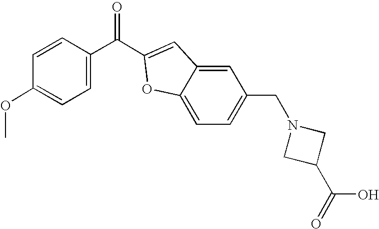 | 1.06 | 366 | M + 1 |
| G.11 | 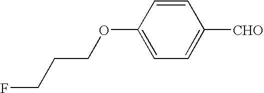 | 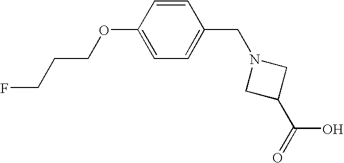 | 0.5 | 268 | M + 1 |
| G.12 | 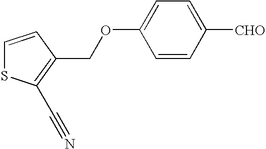 | 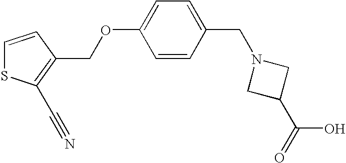 | 0.92 | 329 | M + 1 |
| G.13 | 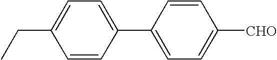 | 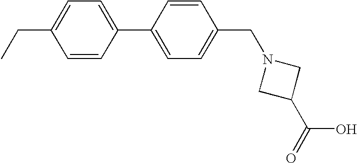 | 1.34 | 296 | M + 1 |
| G.14 | 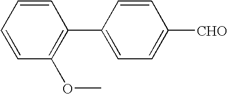 | 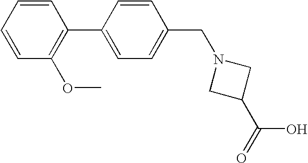 | 1.05 | 298 | M + 1 |
| G.15 | 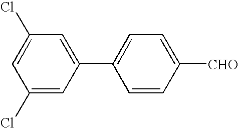 | 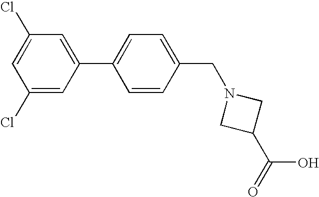 | 1.4 | 336 | M + 1 |
| G.16 | 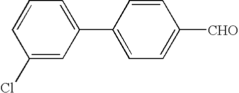 | 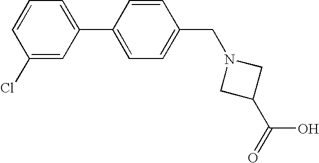 | 1.22 | 302 | M + 1 |

### General Procedure H: Synthesis of Compounds in Table H

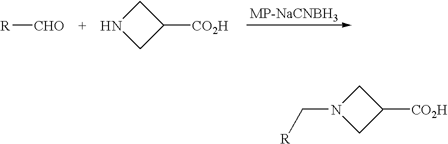

Compounds in Table H were produced as part of a one dimensional array with the only variant being the aldehyde monomer which is given in Table H. The aldehydes were purchased pre-weighed from the Sigma Aldrich Custom Packaged Reagent service.

In a 20 mL vial a solution of the aldehyde monomer (1.2 eq) dissolved in DCM (1.1 mL) was added, followed by the addition of azetidine-3-carboxylic acid (29 mg, 1 eq) dissolved in DCM (0.4 mL), followed by HOAc (3 eq) dissolved in DCM (0.4 mL), followed by MP-cyanoborohydride resin (Biotage, 3 eq). The mixture was shaken at RT for about 5 h. The reaction was checked by LC/MS and concentrated to dryness. The residue was dissolved in 1:1 DMSO:MeOH and purified by preparative HPLC on a Phenomenex Luna C8(2) 5 μm 100 Å AXIA column (30 mm×75 mm) A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the EIC for the target mass exceeded the threshold specified in the method. The system was controlled using Agilent Chemstation (Rev B.10.03), Agilent A2Prep, and Leap FractPal software, with custom Chemstation macros for data export. Products were characterized by MS and LC/MS (Table 2, Method a).

TABLE H

| | | | LC/ | | |
|---|---|---|---|---|---|
| | | | MS | Ob- | M + 1 |
| | | | Rₜ | served | or |
| Ex. # | Starting aldehyde | Structure | (min) | mass | M − 1 |
| H.1 | 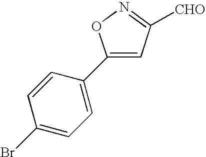 | 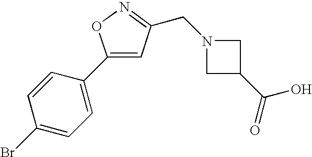 | 1.14 | 337 | M + 1 |
| H.2 | 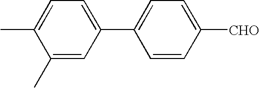 | 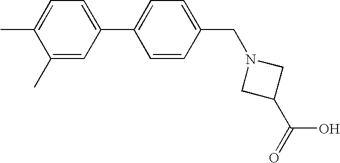 | 1.3 | 296 | M + 1 |
| H.3 | 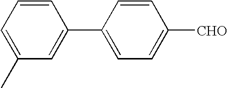 | 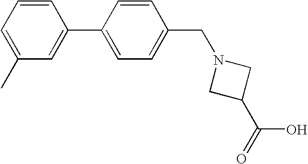 | 1.2 | 282 | M + 1 |
| H.4 | 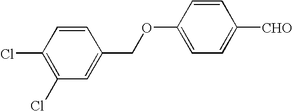 | 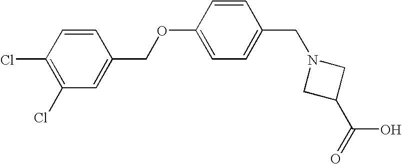 | 1.37 | 366 | M + 1 |
| H.5 | 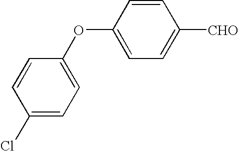 | 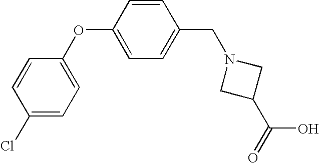 | 1.27 | 318 | M + 1 |
| H.6 | 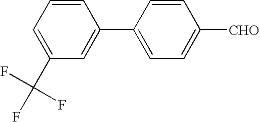 | 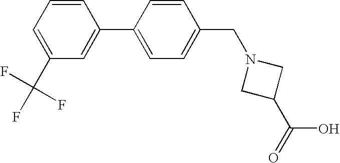 | 1.26 | 336 | M + 1 |
| H.7 | 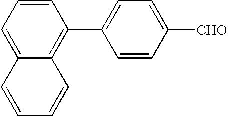 | 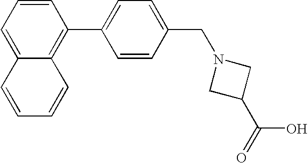 | 1.3 | 318 | M + 1 |
| H.8 | 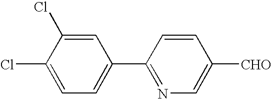 | 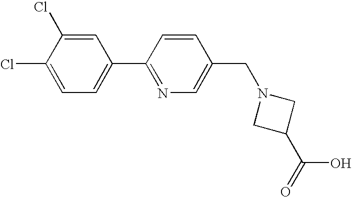 | 1.21 | 336.9 | M + 1 |
| H.9 | 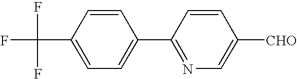 | 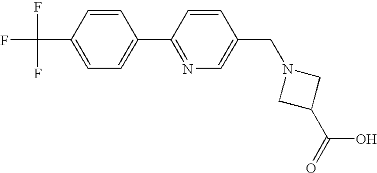 | 1.18 | 337 | M + 1 |

### Example #1

### Preparation of 1-(4-(3,4-Dichlorobenzyloxy)benzyl)-3-methylpiperidine-4-carboxylic acid

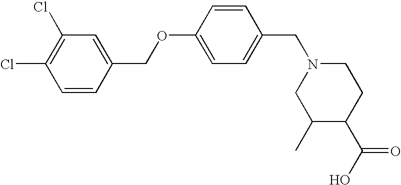

A 20 mL vial was charged with a solution of 4-(3,4-dichlorobenzyloxy)benzaldehyde in DCM (27.60 mg, 1 eq.), a solution of 3-methyl-4-piperidinecarboxylic acid (1.2 eq, 0.6 mmol) in DCM, a solution of HOAc in DCM (5 eq, 21.34 mmol, 30.51 mL), and MP-cyanoborohydride resin (Biotage, 3.0 eq). The vial was capped and placed in a heater/shaker at about 50° C. until reaction was complete. The solvent was removed in vacuo and the crude material was dissolved in 1.4 mL of DMSO:MeOH (1:1 v/v) and purified by preparative HPLC on a Phenomenex Luna C8(2) 5 μm 100 Å AXIA column (30 mm×75 mm). A gradient of acetonitrile (A) and 0.1% trifluoroacetic acid in water (B) was used, at a flow rate of 50 mL/min (0-0.5 min 10% A, 0.5-6.0 min linear gradient 10-100% A, 6.0-7.0 min 100% A, 7.0-8.0 min linear gradient 100-10% A). Samples were injected in 1.5 mL DMSO:MeOH (1:1). An Agilent 1100 Series Purification system was used, consisting of the following modules: Agilent 1100 Series LC/MSD SL mass spectrometer with API-electrospray source; two Agilent 1100 Series preparative pumps; Agilent 1100 Series isocratic pump; Agilent 1100 Series diode array detector with preparative (0.3 mm) flow cell; Agilent active-splitter, IFC-PAL fraction collector/autosampler. The make-up pump for the mass spectrometer used 3:1 MeOH:water with 0.1% formic acid at a flow rate of 1 mL/min. Fraction collection was automatically triggered when the extracted ion chromatogram for the target mass exceeded the threshold specified in the method. The system was controlled using Agilent Chemstation (Rev B.10.03), Agilent A2Prep, and Leap FractPal software, with custom Chemstation macros for data export. 1-(4-(3,4-Dichlorobenzyloxy)benzyl)-3-methylpiperidine-4-carboxylic acid LC/MS (Table 2, Method b) Rₜ: 1.58 min; m/z: 410.0 (M+H)⁺.

### Preparation #1: Preparation of 4-(3,4-Dichlorobenzyloxy)benzaldehyde

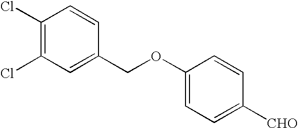

A 2 L round bottomed flask was charged with 4-hydroxybenzaldehyde (150 g, 1.22 mol) and potassium carbonate (254.64 g, 1.84 mol) in acetone (1 L) and 3,4-dichlorobenzyl chloride (240 g, 1.22 mol) was added portion wise. The reaction mixture was then heated to reflux overnight. The reaction completion was monitored by TLC and then cooled to room temperature. The cooled reaction mixture was then poured into a beaker containing cold water to obtain a precipitate. The solid was filtered, washed with water (2×250 mL), dried, then suspended in MeOH (1 L) and stirred for about 15 min at room temperature. The MeOH was filtered off and the solid product was dried to yield 272 g (79%) of 4-(3,4-dichlorobenzyloxy)benzaldehyde. ¹H NMR (DMSO-d₆, 500 MHz): δ 5.22 (s, 2H), 7.2 (d, 2H), 7.43 (m, 1H), 7.69 (d, 1H), 7.78 (s, 1H), 7.90 (d, 2H), 9.85 (s, 1H).

### Example #2

### Preparation of 1-(4-(3,4-Dichlorobenzyloxy)benzyl)azetidine-3-carboxylic Acid

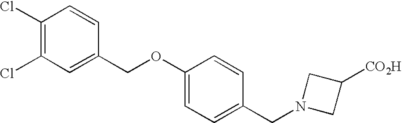

A 3 L round bottomed flask was charged with 4-(3,4-dichlorobenzyloxy)benzaldehyde (80 g, 0.28 mol, Preparation #1) and azetidine-3-carboxylic acid (30.4 g, 0.28 mol) was suspended in MeOH (2 L), then HOAc (8 mL) was added to the reaction mixture and stirred for about 1 h. Sodium cyanoborohydride (9.6 g, 0.15 mol) was added portion wise and the mixture was stirred overnight at RT. The completion of the reaction was monitored by TLC. The solid was filtered, washed with MeOH (2×250 mL) and dried to yield 62 g (60%) of 1-(4-(3,4-dichlorobenzyloxy)benzyl)azetidine-3-carboxylic acid as a white solid. ¹H NMR (500 MHz, DMSO-d₆): δ 3.15 (m, 1H), 3.45 (m, 2H), 3.6 (m, 2H), 3.75 (s, 2H), 5.08 (s, 2H), 6.94 (d, 2H), 7.25 (d, 2H), 7.40 (d, 1H), 7.60 (d, 1H), 7.68 (s, 1H). ¹³C NMR (DMSO-d₆, 125 MHz): δ 33.7, 56.0, 60.8, 67.6, 114.6, 127.7, 129.3, 129.8, 130.3, 130.6, 131.1, 138.3, 157.2, 174.3. IR (KBr pellet): 3421.1, 2923.6, 1612.2, 1512.8, 1248.7 cm⁻¹. LC/MS (Table 1, Method b) Rₜ: 1.81 min; MS m/z: 366.1 (M+H)⁺. MP: 165.6-166.1° C.

### Preparation #2: Preparation of Hexyl 4-formylbenzoate

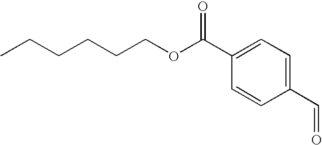

A mixture of 4-formylbenzoic acid (900 mg, 5.99 mmol), 1-bromohexane (0.926 mL, 6.59 mmol) and potassium carbonate (1243 mg, 8.99 mmol) in DMF (12 mL) was heated at about 80° C. overnight. The solid was filtered off and the filtrate was concentrated. The residue was purified by flash chromatography (0-25% EtOAc/heptane over 30 min; Redi-Sep column, 80 g) to give hexyl 4-formylbenzoate (1.128 g, 4.81 mmol, 80% yield) as a brown liquid. %) ¹H NMR (400 MHz, DMSO-d₆) ¹H NMR (400 MHz) δ 10.12 (s, 1H), 8.15 (d, J=8.3, 2H), 8.05 (d, J=8.1, 2H), 4.31 (t, J=6.6, 2H), 1.81-1.64 (m, 2H), 1.47-1.36 (m, 2H), 1.35-1.24 (m, 4H), 0.87 (t, J=7.1, 3H). LC/MS (Table 2, Method b) Rₜ: 2.57 min (no ionization).

### Example #3

### Preparation of 1-(4-(Hexyloxycarbonyl)benzyl)azetidine-3-carboxylic acid

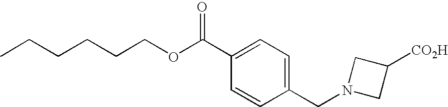

A mixture of hexyl 4-formylbenzoate (50 mg, 0.213 mmol, Preparation #2), azetidine-3-carboxylic acid (25.9 mg, 0.26 mmol) and HOAc (0.061 mL, 1.067 mmol) in MeOH (2 mL) was stirred at about 40° C. overnight. Sodium cyanoborohydride (13.41 mg, 0.213 mmol) was added in one portion and stirred at about 40° C. for about 4 h. The solvent was removed and the residue was purified by mass triggered HPLC to give 1-(4-(hexyloxycarbonyl)benzyl)azetidine-3-carboxylic acid as a white powder (38.8 mg, yield 56.4%) ¹H NMR (400 MHz, DMSO-d₆) δ 7.89 (d, J=8.2, 2H), 7.40 (d, J=8.1, 2H), 4.25 (t, J=6.6, 2H), 3.60 (s, 2H), 3.41-3.32 (m, 2H), 3.21-3.11 (m, 3H), 1.74-1.64 (m, 2H), 1.44-1.34 (m, 2H), 1.30 (dt, J=3.7, 7.1, 4H), 0.87 (t, J=7.1, 3H). LC/MS (Table, 1, Method b) Rₜ: 1.97 min; m/z 320.29 (M+H)⁺.

### Example #4

### Preparation of 1-(4-(Hexyloxy)benzyl)-4-methylpyrrolidine-3-carboxylic acid

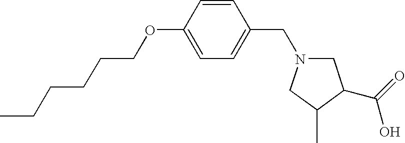

In a 50 mL round-bottomed flask, 4-methylpyrrolidine-3-carboxylic acid (0.4124 g, 3.19 mmol) (Tyger) and 4-(hexyloxy)benzaldehyde (0.659 g, 3.19 mmol) in DCM (15 mL)/MeOH 15 mL were added to give a yellow solution. The MP-cyanoborohydride resin (2.2 mmol/g 1.6 g, 3.51 mmol) (Argonaut) was added in one portion to the solution. The resulting suspension was stirred at about 20° C. overnight. The reaction was filtered and washed with DCM. The filtrate was concentrated to 0.8 g of oil. The oil was dissolved in 3:5 DMSO:MeOH (16 mL) and submitted for purification by mass directed HPLC. The fractions were evaporated to dryness in a Genevac. The fractions were then dissolved in MeOH, evaporated to dryness and dried at about 60° C. in a vacuum oven over the weekend to give 1-(4-(hexyloxy)benzyl)-4-methylpyrrolidine-3-carboxylic acid (0.36 g, 33.5%) as an oil. ¹H NMR (400 MHz, CDCl₃): δ 7.35 (d, J=8.5, 2H), 6.84 (d, J=8.5, 2H), 4.33 (d, J=12.7, 1H), 3.91 (t, J=6.6, 3H), 3.76 (d, J=12.8, 1H), 3.41-3.28 (m, 1H), 2.90 (t, J=10.0, 1H), 2.81 (s, 1H), 2.58 (s, 1H), 2.24 (t, J=10.0, 1H), 1.84-1.68 (m, 2H), 1.43 (s, 2H), 1.38-1.24 (m, 4H), 1.16 (d, J=6.8, 3H), 0.88 (t, J=6.7, 3H). LC/MS (Table 1, Method b) Rₜ: 1.85 min; MS m/z: 320.2 (M+H)⁺.

### Example #5

### Preparation of 1-(4-(3,4-Dichlorobenzyloxy)-3-nitrobenzyl)azetidine-3-carboxylic acid

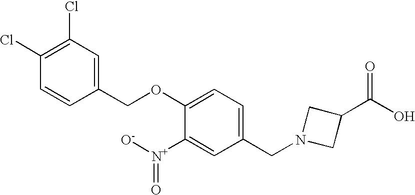

In a 100 mL round-bottomed flask, azetidine-3-carboxylic acid (0.3 g, 2.97 mmol) and 4-(3,4-dichlorobenzyloxy)-3-nitrobenzaldehyde (0.968 g, 2.97 mmol) (Bionet) in MeOH:DCM (1:1, 30 mL) were added to give a yellow suspension. MP-cyanoborohydride 2.2 mmol/g (1.6 g, 2.97 mmol) (Argonaut) was added in one portion to the suspension. The resulting suspension was stirred at about 20° C. overnight. The reaction was filtered and washed with DCM. The filtrate was concentrated to give 0.5 g of foam. The foam was dissolved in DMSO (6 mL) and MeOH (6 mL), submitted for purification by mass directed HPLC. The fractions were evaporated to dryness in a Genevac overnight and then dried at about 63° C. in a vacuum oven over the weekend to give 1-(4-(3,4-dichlorobenzyloxy)-3-nitrobenzyl)azetidine-3-carboxylic acid (0.218 g, 18%) as a white solid. ¹H NMR (400 MHz, CD₃CN) δ 7.77 (d, J=2.1, 1H), 7.65 (d, J=1.9, 1H), 7.56 (d, J=8.3, 1H), 7.51 (dd, J=2.2, 8.6, 1H), 7.44-7.36 (m, 1H), 7.21 (d, J=8.6, 1H), 5.20 (s, 2H), 3.59 (s, 2H), 3.45 (t, J=7.6, 2H), 3.30 (t, J=6.7, 2H), 3.24 (dd, J=7.2, 13.9, 1H). LC/MS (Table 1, Method b) Rₜ: 1.83 min; MS m/z: 411.02 (M+H)⁺.

### Example #6

### Preparation of 1-(4-(Hexyloxy)-3-methoxybenzyl)azetidine-3-carboxylic acid

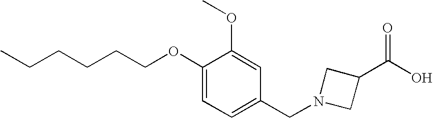

In a 200 mL round-bottomed flask, 4-(hexyloxy)-3-methoxybenzaldehyde (2.5 g, 10.58 mmol) (Enamine) and azetidine-3-carboxylic acid (1.1 g, 10.58 mmol) in DCM (25 mL) and MeOH (25 mL) were added to give a yellow suspension. After stirring for about 30 min at room temperature sodium cyanoborohydride (0.731 g, 11.64 mmol) was added in one portion. The reaction was stirred at room temperature overnight. LC/MS indicated nearly complete conversion to the desired product. The solvents were removed under reduced pressure and the crude material was brought up in DCM, and washed with water. Upon addition of water to the DCM the entire mixture turned to an opaque solution. After about 2 h, the mixture separated. The DCM layer was collected, dried (MgSO₄), filtered and concentrated in vacuo to yield a pale yellow oil. Ether was added, and the crude material dissolved completely. 1.0 M HCl in ether was added drop-wise until the solution turned cloudy and precipitate formed. The material was filtered to collect the white solid, the filter cake was washed with ether (3×25 mL) and dried in a vacuum oven overnight to yield 1-(4-(hexyloxy)-3-methoxybenzyl)azetidine-3-carboxylic acid (1.48 g, 39%) as a white solid. ¹H NMR (400 MHz, d₄-MeOH): δ ppm 7.09 (s, 1H), 7.00 (q, J=8.27 Hz, 2H), 4.36-4.21 (m, 6H), 4.00 (t, J=6.46 Hz, 2H), 3.87 (s, 3H), 3.65 (m, 1H), 1.87-1.70 (m, 2H), 1.55-1.41 (m, 2H), 1.41-1.27 (m, 4H), 0.92 (t, J=6.43 Hz, 3H). LC/MS (Table 1, Method b) Rₜ: 1.61 min; MS m/z: 322.25 (M+H)⁺.

### Preparation #3: Synthesis of 4-(1,3-Dioxolan-2-yl)benzonitrile

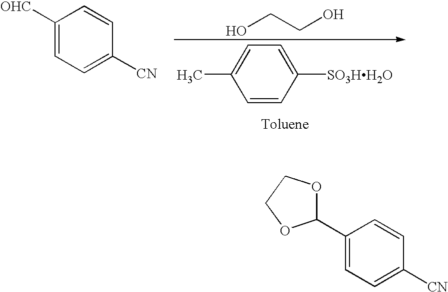

A mixture of 4-formylbenzonitrile (13.1 g, 0.1 mol), ethylene glycol (62 g, 1 mol), p-toluenesulfonic acid monohydrate (1.9 g, 0.01 mol) in 150 mL of toluene were refluxed overnight. After cooling the mixture to room temperature, it was added to 200 mL of ice-cold water and stirred for about 15 min. The organic layer was dried (Na₂SO₄), filtered and the solvent was removed in vacuo. Purification on silica-gel column chromatography (PE:EtOAc from 10:1 to 4:1) afforded 4-(1,3-dioxolan-2-yl)benzonitrile as a white solid (14.2 g, yield 81%). LC/MS (Table 1, Method c) Rₜ: 0.66 min; m/z 176.7 (M+H)⁺.

### Preparation #4: Synthesis of 4-(2-Phenylacetyl)benzaldehyde

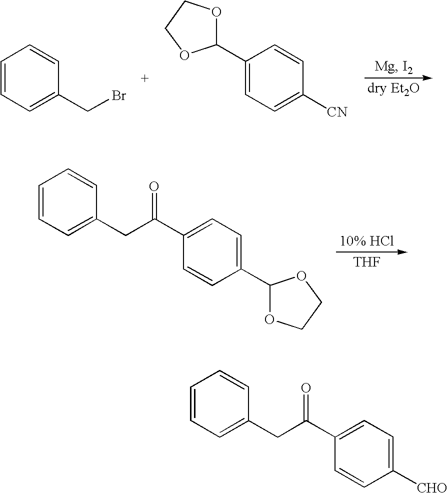

Under N₂, to magnesium turnings (792 mg, 32.98 mmol) and I₂ (7 mg) in Et₂O (5 mL), was added a solution of (bromomethyl)benzene (3.76 g, 21.99 mmol) at room temperature. After stirring for about 1 h, the mixture was cooled to 0-15° C. A solution of 4-(1,3-dioxolan-2-yl)benzonitrile (2.89 g, 16.5 mmol) in Et₂O (10 mL) was added dropwise, then the mixture was refluxed for about 1 h. The solution was cooled to room temperature and treated with ice-water. Subsequently, aqueous 5 M HCl was added. The organic phase was separated and the aqueous phase was extracted with EtOAc. The combined organic phase was washed with saturated NaHSO₃ and saturated NaHCO₃, dried (Na₂SO₄) and concentrated in vacuo to get crude 1-(4-(1,3-dioxolan-2-yl)phenyl)-2-phenylethanone. The crude 1-(4-(1,3-dioxolan-2-yl)phenyl)-2-phenylethanone was dissolved in THF (20 mL) and 10% HCl (30 mL) added to the solution. The reaction mixture was refluxed for about 16 h, then cooled to room temperature. EtOAc was added and the organic layer dried (Na₂SO₄) and filtered. After removal of the solvent, the residue was purified by silica-gel column chromatography (PE:EtOAc from 50:1 to 10:1) to afford 4-(2-phenylacetyl)benzaldehyde as a white solid (1.1 g, yield 43%). LC/MS (Table 2, Method c) Rₜ: 1.57 min; m/z 225.1 (M+H)⁺.

### Example #7

### Synthesis of 1-(4-(2-Phenylacetyl)benzyl)azetidine-3-carboxylic acid

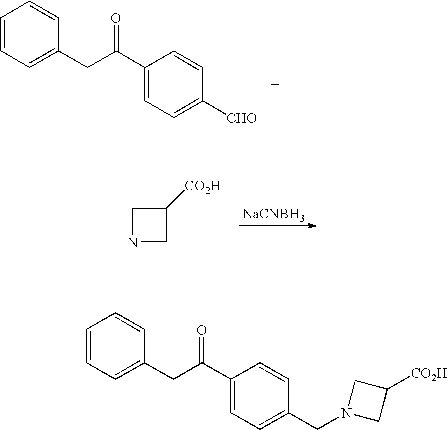

4-(2-Phenylacetyl)benzaldehyde (336 mg, 1.5 mmol, Preparation #4) was added to a stirred solution of azetidine-3-carboxylic acid (152 mg, 1.5 mmol) and HOAc (270 mg, 4.5 mmol) in MeOH (5 mL). The mixture was heated to about 40° C. After about 15 min, NaCNBH₃ (279 mg, 4.5 mmol) was added in a single portion and stirred at about 40° C. for about 24 h. After acidifying with 1M HCl, the residue was purified by Prep-HPLC affording 1-(4-(2-phenylacetyl)benzyl)azetidine-3-carboxylic acid (10 mg, yield 11%). LC/MS (Table 2, Method c) Rₜ: 1.23 min; m/z 310.2 (M+H)⁺.

### Example #8

### Synthesis of 1-(4-Phenethylbenzyl)azetidine-3-carboxylic acid

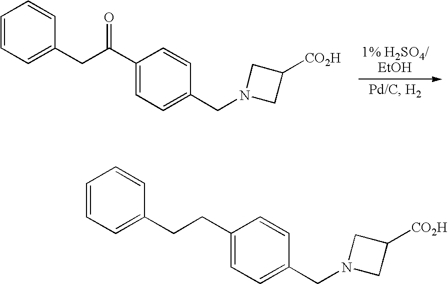

1-(4-(2-Phenylacetyl)benzyl)azetidine-3-carboxylic acid (200 mg, 0.65 mmol, Example #7) was dissolved in 1% (v/v) H₂SO₄:EtOH (50 mL), to which Pd/C (10 mg) was added and the hydrogenation reaction carried out at RT for about 32 h. After completion of the reaction, the catalyst was filtered off and ethanol solution neutralized with NaOH, followed by distillation of the solvent. HPLC afforded 1-(4-phenethylbenzyl)azetidine-3-carboxylic acid (100 mg, 52%). ¹H NMR (500 MHz, d₄-MeOH): δ 7.24-7.23 (2H, d), 7.18-7.16 (2H, d), 7.13-7.10 (2H, t), 7.05-7.03 (2H, t), 7.02 (1H, m), 4.25 (2H, s), 4.19-4.18 (2H, m), 4.17-4.15 (2H, m), 3.22-3.20 (1H, m), 2.87-2.84 (2H, m), 2.82-2.79 (2H, m). LC/MS (Table 1, Method c) Rₜ: 1.35 min; m/z 296.2 (M+H)⁺.

### Preparation #5: Synthesis of 4-(2-(3-(Trifluoromethyl)phenyl)acetyl)benzaldehyde

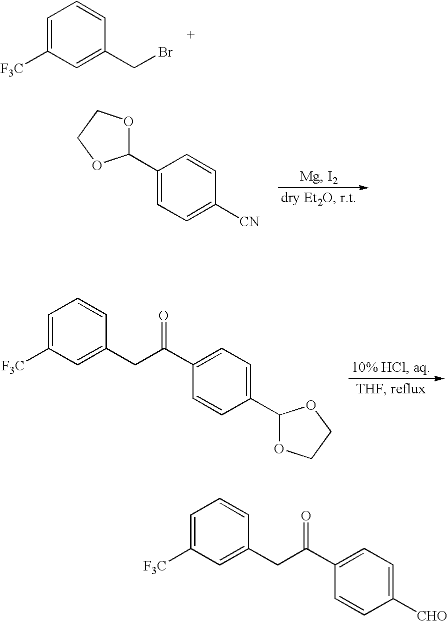

To magnesium turnings (0.90 g, 37.5 mmol) and I₂ (12 mg) in Et₂O (10 mL), was added a solution of 3-(trifluoromethyl)benzyl bromide (5.95 g, 25.0 mmol) at RT under nitrogen atmosphere. After stirring for about 1 h, the mixture was cooled to 0-15° C. A solution of 4-(1,3-dioxolan-2-yl)benzonitrile (3.29 g, 18.8 mmol) in Et2O (10 mL) was added dropwise, then the mixture was refluxed for 1 h. The solution was cooled to RT and treated with ice-water. Aqueous 5 M HCl was added, the organic phase separated and the aqueous phase was extracted with EtOAc. The combined organic phase was washed with saturated NaHSO₃ and saturated NaHCO₃, dried (Na₂SO₄), filtered and concentrated in vacuo to get the crude 1-(4-(1,3-dioxolan-2-yl)phenyl)-2-(3-(trifluoromethyl)phenyl)ethanone. The crude 1-(4-(1,3-dioxolan-2-yl)phenyl)-2-(3-(trifluoromethyl)phenyl)ethanone was dissolved in THF (20 mL) and 10% HCl (30 mL) was added to the solution. The reaction mixture was refluxed for about 16 h, then cooled to RT. EtOAc was added and the organic layer was dried with Na₂SO₄. After removal of the solvent, it was purified by silica-gel column chromatography (PE/EA from 50:1 to 10:1) to afford 4-(2-(3-(trifluoromethyl)phenyl)acetyl)benzaldehyde as a white solid (1.66 g, yield 30%). LC/MS (Table 2, Method c): Rₜ: 1.67 min, 293.1 m/z (M+H)⁺.

### Preparation #6: Synthesis of 1-(4-(2-(3-(Trifluoromethyl)phenyl)acetyl)benzyl)azetidine-3-carboxylic acid

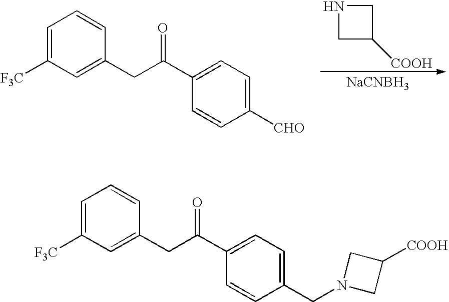

4-(2-(3-(Trifluoromethyl)phenyl)acetyl)benzaldehyde (1.17 g, 4 mmol, Preparation #5) was added to a stirred solution of azetidine-3-carboxylic acid (0.40 g, 4 mmol) and HOAc (0.72 g, 12 mmol) in 30 mL CH₃OH. The mixture was heated to about 40° C. After about 15 min, NaCNBH₃ (0.76 g, 12 mmol) was added in a single portion and stirred at about 40° C. for about 24 h. After acidification with 1M HCl, purification by Prep-HPLC gave 1-(4-(2-(3-(trifluoromethyl)phenyl)acetyl)benzyl)azetidine-3-carboxylic acid (0.40 g, yield 26%). LC/MS (Table 2, Method c): Rₜ: 1.36 min, m/z 378.1 (M+H)⁺.

### Example #9

### Synthesis of 1-(4-(3-(Trifluoromethyl)phenethyl)benzyl)azetidine-3-carboxylic Acid

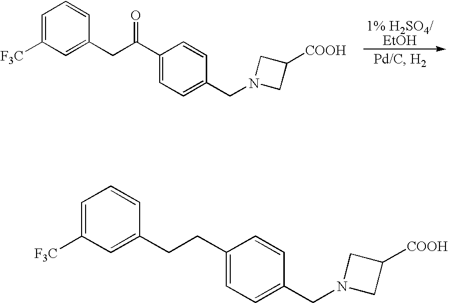

1-(4-(2-(3-(Trifluoromethyl)phenyl)acetyl)benzyl)azetidine-3-carboxylic acid (0.4 g, 1.06 mmol, Preparation #6) was dissolved into 50 mL 1% (v/v) H₂SO₄/EtOH, to the resultant solution was added Pd/C (40 mg) and the hydrogenation reaction was carried out at room temperature for about 32 h using a hydrogen balloon. After completion of the reaction, the catalyst was filtered off and ethanol solution was neutralized with aqueous NaOH, followed by distillation of the solvent. Purification by Prep-HPLC gave 1-(4-(3-(trifluoromethyl)phenethyl)benzyl)azetidine-3-carboxylic acid (0.21 g, 54%). ¹H NMR: (500 MHz, d₄-MeOH): δ 7.44-7.37 (m, 4H), 7.17 (d, 2H, J=8.0 Hz), 7.10 (d, 2H, J=8.0 Hz), 3.58 (s, 2H), 3.50 (t, 2H, J=7.8 Hz), 3.32 (t, 2H, J=8.5 Hz), 3.21-3.14 (m, 1H), 2.99-2.88 (m, 4H). LC/MS (Table 2, Method c): Rₜ: 0.63 min, m/z 364.2 (M+H)⁺.

### Preparation #7: Synthesis of 4-(Benzyloxy)-3-fluorobenzonitrile

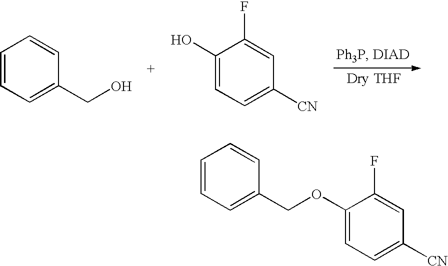

Under N₂, DIAD (0.32 g, 1.54 mmol) was treated with Ph₃P (0.41 g, 4.54 mmol) at about 0° C. in dry THF (10 mL). The mixture was stirred until there was a precipitate. Then 3-fluoro-4-hydroxybenzonitrile (0.2 g, 1.46 mmol) and benzyl alcohol (0.17 g, 1.54 mmol) were added at the same time. The mixture was warmed to RT, and stirred overnight. The reaction mixture was concentrated and purified by silica-gel column chromatography (PE:EtOAc=4:1) to afford 4-(benzyloxy)-3-fluorobenzonitrile as a white solid (0.28 g, yield 84%). GC/MS (Table 2, Method d) Rₜ: 10.83 min; m/z 227.1 (M).

### Example #I.1

### Preparation of 1-(4-(benzyloxy)-3-fluorobenzyl)azetidine-3-carboxylic acid

### Step A: Preparation #8: Synthesis of 4-(Benzyloxy)-3-fluorobenzaldehyde

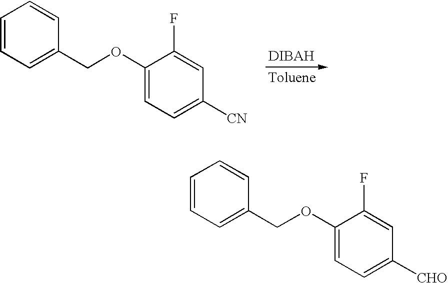

4-(Benzyloxy)-3-fluorobenzonitrile (645 mg, 2.84 mmol, Preparation #7) was dissolved in toluene (10 mL) and cooled to about 0° C. A portion of 1M DIBAH (4.55 mmol, 4.55 mL) in hexane was added dropwise under N₂. The solution was stirred for about 1 h at about 0° C. Chloroform (12 mL) was then added followed by 10% HCl (30 mL), and the resulting solution stirred at RT for about 1 h. The organic layer was separated, washed with distilled water, dried (Na₂SO₄) and filtered. After removal of the solvent, the residue was purified by silica-gel column chromatography (PE:EtOAc=4:1) to afford 4-(benzyloxy)-3-fluorobenzaldehyde as a white solid (0.62 g, yield 95%). LC/MS (Table 2, Method c) Rₜ: 1.26 min; m/z 231.1 (M+H)⁺.

### Step B: Synthesis of 1-(4-(benzyloxy)-3-fluorobenzyl)azetidine-3-carboxylic acid

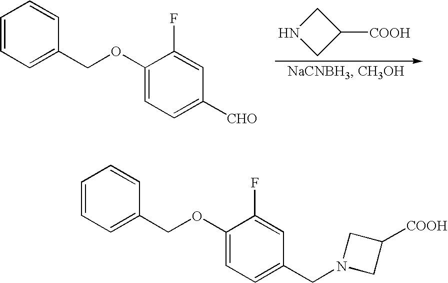

4-(Benzyloxy)-3-fluorobenzaldehyde (653 mg, 2.84 mmol) was added to a stirred solution of azetidine-3-carboxylic acid (287 mg, 2.84 mmol) and HOAc (536 mg, 8.52 mmol) in 5 mL CH₃OH. The mixture was heated to about 40° C. After about 15 min, NaCNBH₃ (512 mg, 8.52 mmol) was added in a single portion and stirred at about 40° C. overnight. After acidifying with 1M HCl, the residue was purified by HPLC to afford 1-(4-(benzyloxy)-3-fluorobenzyl)azetidine-3-carboxylic acid (571 mg, yield 63%). ¹H NMR (500 MHz, d₄-MeOH): δ 7.44-7.43 (2H, d), 7.39-7.36 (2H, m), 7.33-7.30 (1H, m), 7.28-7.25 (1H, m), 7.23-7.22 (1H, d), 7.20-7.18 (1H, m), 5.19 (2H, s), 4.32 (2H, s), 4.29-4.25 (4H, m), 3.68-3.63 (1H, m). LC/MS (Table 2, Method c) Rₜ: 0.95 min; m/z 316.2 (M+H)'.

Compounds in Table I were prepared using the same procedure as for 1-(4-(benzyloxy)-3-fluorobenzyl)azetidine-3-carboxylic acid, Example #I.1.

TABLE I

| | | | | LC/ | | | |
|---|---|---|---|---|---|---|---|
| | Starting | | | MS | Ob- | M + 1 | |
| Ex. | benzyl | Starting | | Rₜ | served | or | |
| # | alcohol | benzonitrile | Structure | (min) | mass | M − 1 | NMR |
| I.2 | 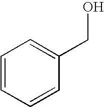 | 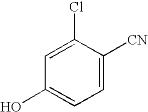 | 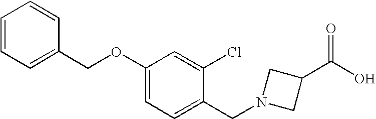 | 1.01 | 332.1 | M + 1 | ¹H NMR (500 MHz, d₄-MeOH): δ 7.51-7.49 (1H, d), 7.46-7.44 (2H, d), 7.41-7.38 (2H, q), 7.35- 7.32 (1H, q), 7.21-7.20 (1H, d), 7.08-7.06 (1H, q), 5.15 (2H, s), 4.45 (2H, s), 4.22-4.18 (4H, m), 3.46-3.41 (1H, m) |
| I.3 | 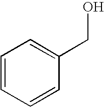 | 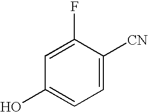 | 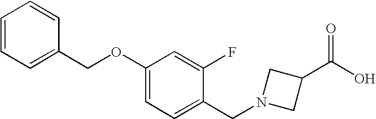 | 0.97 | 316.1 | M + 1 | ¹H NMR (500 MHz, d₄-MeOH): δ 7.43-7.31 (6H, m), 6.92-6.89 (m, 2H), 5.12 (s, 2H), 4.35 (s, 2H), 4.20 (d, 4H, J = 8.5 Hz), 3.45- 3.42 (m, 1H). |
| I.4 | 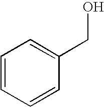 | 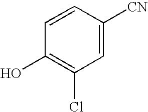 | 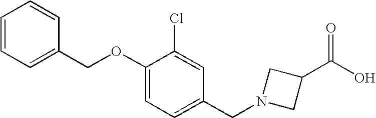 | 1.01 | 332.1 | M + 1 | ¹H NMR (500 MHz, d₄-MeOH): δ 7.57-7.56 (1H, d), 7.49-7.47 (2H, d), 7.41-7.32 (4H, m), 7.24- 7.22 (1H, d), 5.24 (2H, s), 4.33 (2H, s), 4.30-4.26 (4H, s), 3.33- 3.32 (1H, m) |
| I.5 | 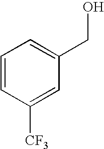 | 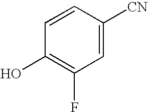 | 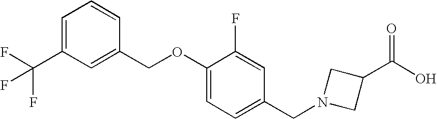 | 1.08 | 384.2 | M + 1 | ¹H NMR (500 MHz, d₄-MeOH): δ 7.76 (1H, s), 7.73-7.71 (1H, d), 7.65-7.64 (1H, d), 7.61-7.58 (1H, t), 7.31-7.28 (1H, m), 7.27- 7.25 (1H, d), 7.23-7.21 (1H, m), 5.23 (2H, s), 4.33 (2H, s), 4.30- 4.26 (4H, m), 3.69-3.63 (1H, m) |
| I.6 | 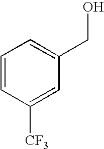 | 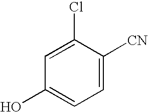 | 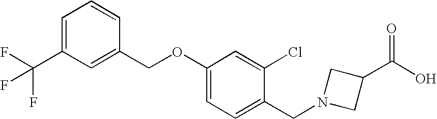 | 1.12 | 400.1 | M + 1 | ¹H NMR (500 MHz, d₄-MeOH): δ 7.66 (1H, s), 7.62-7.61 (1H, d), 7.55-7.53 (1H, d), 7.50-7.47 (1H, t), 7.43-7.42 (1H, d), 7.15- 7.14 (1H, d), 7.01-6.98 (1H, q), 5.12 (2H, s), 4.44 (2H, s), 4.30- 4.23 (4H, m), 3.62-3.58 (1H, m) |
| I.7 | 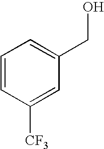 | 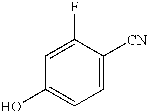 | 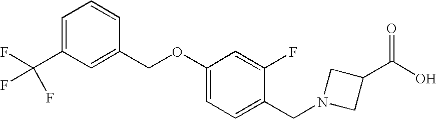 | 1.08 | 384.2 | M + 1 | ¹H NMR (500 MHz, d₄-MeOH): δ 7.75 (1H, s), 7.72-7.70 (1H, d), 7.65-7.63 (1H, d), 7.60-7.57 (1H, t), 7.45-7.42 (1H, t), 6.98- 6.96 (2H, q), 5.22 (2H, s), 4.42 (2H, s), 4.36-4.29 (4H, m), 3.70- 3.63 (1H, m) |
| I.8 | 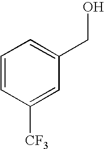 | 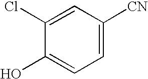 | 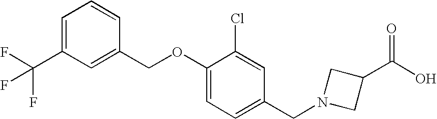 | 1.12 | 400.1 | M + 1 | ¹H NMR (500 MHz, d₄-MeOH): δ 7.82 (1H, s), 7.77-7.76 (1H, d), 7.67-7.65 (1H, d), 7.63-7.60 (2H, q), 7.42-7.40 (1H, q), 7.27- 7.26 (1H, d), 5.33 (2H, s), 4.36 (2H, s), 4.33-4.28 (4H, m), 3.72- 3.66 (1H, m) |
| I.9 | 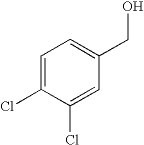 | 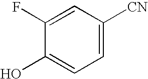 | 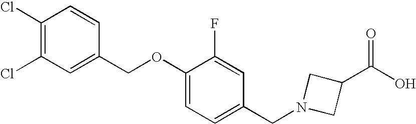 | 1.11 | 384.1 | M + 1 | ¹H NMR (500 MHz, d₄-MeOH): δ 7.64 (1H, s), 7.57-7.55 (1H, d), 7.41-7.40 (1H, d), 7.31-7.29 (1H, d), 7.24-7.23 (2H, q), 5.21 (2H, s), 4.31 (2H, s), 4.22-4.21 (4H, d), 3.50-3.33 (1H, m) |
| I.10 | 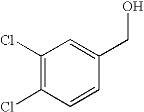 | 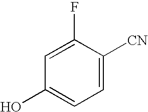 | 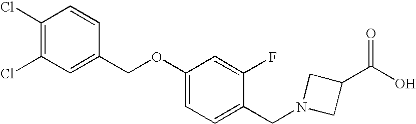 | 1.1 | 384.1 | M + 1 | ¹H NMR (500 MHz, d₄-MeOH): δ 7.52 (1H, s), 7.45-7.44 (1H, d), 7.36-7.32 (1H, m), 7.29-7.27 (1H, d), 6.87-6.84 (2H, m), 5.04 (2H, s), 4.33 (2H, s), 4.27-4.20 (4H, m), 3.59-3.56 (1H, m) |

### Preparation #9: Synthesis of 1-Bromo-4-(3,4-dichlorobenzyloxy)-2-methylbenzene

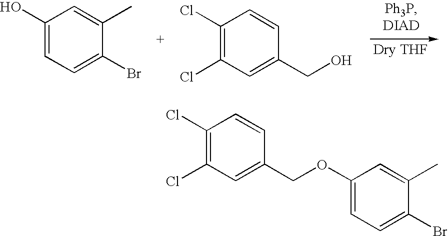

Under N₂, DIAD (908 mg, 4.49 mmol) was treated with Ph₃P (1.18 g, 4.49 mmol) at about 0° C. in dry THF (20 mL). The mixture was stirred until a precipitate formed. 4-Bromo-3-methylphenol (0.8 g, 4.23 mmol) and 3,4-dicholorobenzyl alcohol (795 mg, 4.49 mmol) were added at the same time. The mixture was warmed to room temperature and stirred overnight. After concentrating, purification by silica-gel column chromatography (PE:EtOAc=4:1) afforded 1-bromo-4-(3,4-dichlorobenzyloxy)-2-methylbenzene as a white solid (0.94 g, yield 64%). ¹H NMR (500 MHz, CDCl₃): δ 7.52 (1H, s), 7.46-7.44 (1H, d), 7.42-7.40 (1H, d), 7.25-7.23 (1H, q), 6.85-6.84 (1H, d), 6.66-6.64 (1H, q), 4.97 (2H, s), 2.36 (3H, s). GCMS (Table 2, Method d) Rₜ: 13.96 min; m/z 345.9 (M).

### Preparation #10: Synthesis of 4-(3,4-Dichlorobenzyloxy)-2-methylbenzonitrile

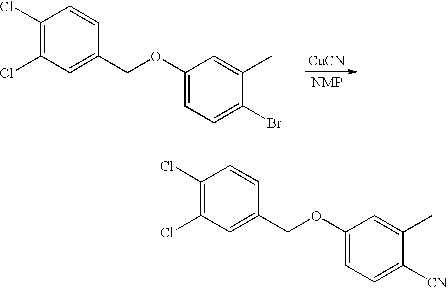

A mixture of 1-bromo-4-(3,4-dichlorobenzyloxy)-2-methylbenzene (0.94 g, 207 mmol, Preparation #9) and CuCN (0.56 g, 6.2 mmol) in N-methyl-2-pyrrolidone (10 mL) was heated to about 160° C. for about 32 h. After cooling slightly, the mixture was washed with 5% aqueous NaCN and extracted with ether. The combined extract was washed with 5% aqueous NaCN, water, brine and dried (Na₂SO₄). After removal of solvent in vacuo, the residue was purified by silica-gel column chromatography (PE:EtOAc=10:1) to afford 4-(3,4-dichlorobenzyloxy)-2-methylbenzonitrile as a white solid (496 mg, yield 63%). LC/MS (Table 2, Method c) Rₜ: 1.46 min; m/z 292.0 (M+H)⁺.

### Example #J.1

### Exemplification of General Procedure J

### Preparation of 1-(4-(3,4-Dichlorobenzyloxy)-2-methylbenzyl)azetidine-3-carboxylic acid

### Step A: Preparation #11

### Synthesis of 4-(3,4-Dichloro-2-methylbenzyloxy)benzaldehyde

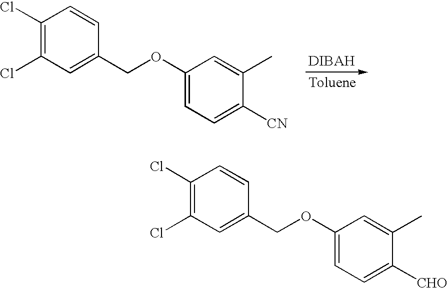

4-(3,4-Dichlorobenzyloxy)-2-methylbenzonitrile (644 mg, 2.2 mmol, Preparation #10) was dissolved in toluene (35 mL) and cooled to about 0° C. A portion of 1M DIBAH (3.5 mmol, 3.5 mL) in hexane was added dropwise under N₂. The solution was stirred for about 1 h at about 0° C. CHCl₃ (40 mL) was then added followed by 10% HCl (30 mL), and the solution was stirred at room temperature for about 1 h. The organic layer was separated, washed with distilled water, dried (Na₂SO₄) and filtered. After removal of the solvent, the residue was purified by silica-gel column chromatography (PE:EtOAc=4:1) to afford 4-(3,4-dichloro-2-methylbenzyloxy)benzaldehyde as a white solid (345 mg, yield 53%). LC/MS (Table 2, Method c) Rₜ: 1.85 min; m/z 295.0 (M+H)⁺.

### Step B: Synthesis of 1-(4-(3,4-Dichlorobenzyloxy)-2-methylbenzyl)azetidine-3-carboxylic acid

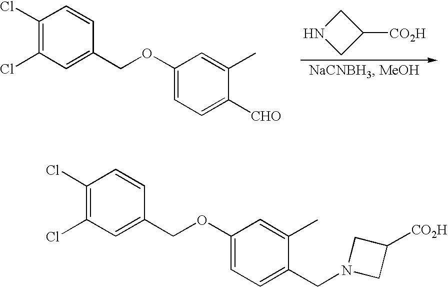

4-(3,4-Dichloro-2-methylbenzyloxy)benzaldehyde (Preparation #11) (345 mg, 1.17 mmol) was added to a stirred solution of azetidine-3-carboxylic acid (118 mg, 1.17 mmol) and HOAc (211 mg, 3.51 mmol) in 5 mL CH₃OH. The mixture was heated to about 40° C. After about 15 min, NaCNBH₃ (218 mg, 3.51 mmol) was added in a single portion and stirred at about 40° C. overnight. After acidifying with 1M HCl the residue was purified by HPLC to afford 1-(4-(3,4-dichlorobenzyloxy)-2-methylbenzyl)azetidine-3-carboxylic acid (201 mg, yield 45%). ¹H NMR (500 MHz, d₄-MeOH): δ 7.59 m, 1H), 7.51 (d, 1H, J=7.5 Hz); 7.35 (dd, 1H, J1=2.0 Hz, J2=8.0 Hz), 7.31 (d, 1H, J=8.5 Hz), 6.94 (m, 1H), 6.89 (dd, 1H, J1=2.5 Hz, J2=8.5 Hz), 5.08 (s, 2H), 4.35 (s, 2H), 4.18 (d, 4H, J=8.5 Hz), 3.45-3.39 (m, 1H), 2.39 (s, 3H). LC/MS (Table 1, Method c) Rₜ: 1.47 min; m/z 380.1 (M+H)⁺.

Compounds in Table J were prepared using the same procedure as for 1-(4-(3,4-dichlorobenzyloxy)-2-methylbenzyl)azetidine-3-carboxylic acid, Example #J.1.

TABLE J

| | Starting | Starting | | LC/MS | Ob- | M + 1 | |
|---|---|---|---|---|---|---|---|
| Ex. | benzyl | benzo- | | Rₜ | served | or | |
| # | alcohol | nitrile | Product | (min) | mass | M − 1 | NMR |
| J.2 | 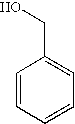 | 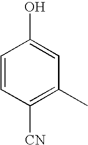 | 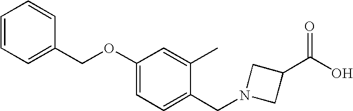 | 1.32 | 312.2 | M + 1 | ¹H NMR (500 MHz, d₄-MeOH): δ 7.45-7.43 (2H, d), 7.40-7.37 (2H, t), 7.34-7.31 (2H, t), 6.98 (1H, s), 6.94-6.92 (1H, q), 5.12 (2H, s), 4.42 (2H, s), 4.35-4.29 (4H, m), 3.73-3.66 (1H, m), 2.42 (3H, s) |
| J.3 | 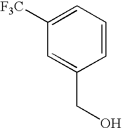 | 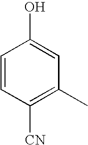 | 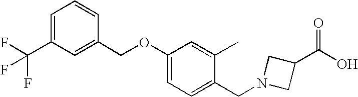 | 1.43 | 380.2 | M + 1 | ¹H NMR (500 MHz, d₄-MeOH): δ 7.59 (1H, s), 7.56-7.54 (1H, d), 7.48-7.46 (1H, d), 7.44-7.41 (1H, t), 7.19-7.17 (1H, d), 6.82 (1H, d), 6.82-6.76 (1H, q), 5.03 (2H, s), 4.22 (2H, s), 4.06-4.05 (4H, d), 3.30-3.29 (1H, m), 2.25 (3H, s) |

### Preparation #12: Synthesis of 4-(1-Phenylethoxy)benzonitrile

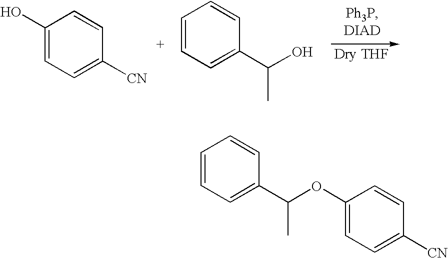

Under N₂, DIAD (1.06 g, 5.25 mmol) was treated with Ph₃P (1.38 g, 5.25 mmol) at about 0° C. in dry THF (15 mL). The mixture was stirred until a precipitate formed. 4-Hydroxybenzonitrile (630 mg, 5.25 mmol) and DL-1-phenylethanol (611 mg, 5 mmol) were added at the same time. The mixture was warmed to room temperature and stirred overnight. The solution was concentrated and purified by silica-gel column chromatography (PE:EtOAc from 15:1 to 10:1) to afford 4-(1-phenylethoxy)benzonitrile as a white solid (0.62 g, yield 55%). ¹H NMR (500 MHz, CDCl₃): δ 7.48 (d, 2H, J=7.5 Hz); 7.36-7.32 (m, 4H), 7.29 (t, 1H, J=7.0 Hz), 6.90 (d, 2H, J=8.5 Hz); 5.36-5.33 (q, 1H), 1.66 (d, 3H, J=6.5 Hz). GCMS (Table 2, Method d) Rₜ: 11.05 min; m/z 223.1 (M).

### Preparation #13: Synthesis of 4-(1-Phenylethoxy)benzaldehyde

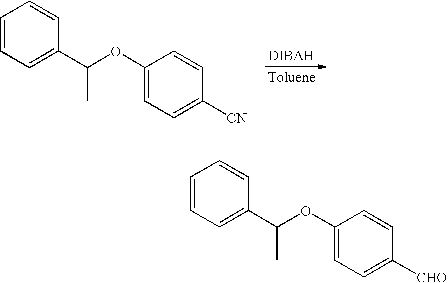

4-(1-Phenylethoxy)benzonitrile (Preparation #12) (200 mg, 0.86 mmol) was dissolved in toluene (10 mL) and cooled to about 0° C. 1M DIBAH (1.44 mmol, 1.44 mL) in hexane was added dropwise under N₂. The solution was stirred for about one hour at about 0° C. CHCl₃ (12 mL) was then added followed by 10% HCl (30 mL), and the solution was stirred at room temperature for about 1 h. The organic layer was separated, washed with distilled water, (Na₂SO₄), filtered and concentrated in vacuo. Purification by silica-gel column chromatography (PE:EtOAc=4:1) afforded 4-(1-phenylethoxy)benzaldehyde as a white solid (85 mg, yield 42%). LC/MS (Table 2, Method c) Rₜ: 2.17 min; m/z 227 (M+H)⁺.

### Example #10

### Synthesis of 1-(4-(1-Phenylethoxy)benzyl)azetidine-3-carboxylic acid

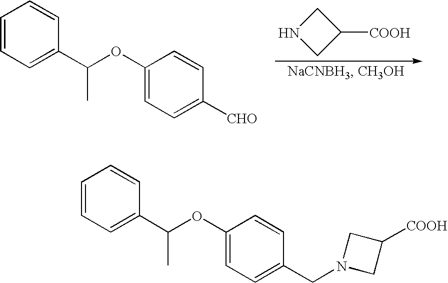

4-(1-Phenylethoxy)benzaldehyde (Preparation #13) (1.06 g, 4.7 mmol) was added to a stirred solution of azetidine-3-carboxylic acid (476 mg, 4.7 mmol) and HOAc (850 mg, 14.1 mmol) in 5 mL CH₃OH. The mixture was heated to about 40° C. After about 15 min, NaCNBH₃ (889 mg, 14.1 mmol) was added in a single portion and stirred at about 40° C. overnight to afford 1-(4-(1-phenylethoxy)benzyl)azetidine-3-carboxylic acid (400 mg, yield 27%). ¹H NMR (500 MHz, d₄-MeOH): δ 7.27-7.26 (2H, d), 7.22-7.16 (4H, m), 7.17-7.11 (1H, t), 6.85-6.83 (2H, d), 5.34-5.31 (1H, q), 4.11 (2H, s), 4.03-4.02 (4H, d), 4.32-3.25 (1H, m), 1.51-1.49 (3H, d). LC/MS (Table 2, Method c) Rₜ: 1.00 min; m/z 312.1 (M+H)⁺.

### Preparation #14: Synthesis of (R)-1-(4-(1-Phenylethoxy)benzyl)azetidine-3-carboxylic acid

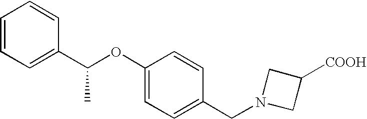

(R)-1-(4-(1-Phenylethoxy)benzyl)azetidine-3-carboxylic acid was synthesized using the same procedure as 1-(4-(1-phenylethoxy)benzyl)azetidine-3-carboxylic acid starting from the chiral material (S)-1-phenylethanol. The configuration conversion ratio for the Mitsunobu step was 86:14(R:S). The compound was purified by Chiral-HPLC using an OJ-H column and hexane:ethanol=75%:25% as eluent and used as standard to confirm the configuration of the other enantiomer (S)-1-(4-(1-phenylethoxy)benzyl)azetidine-3-carboxylic acid. ¹H NMR (500 MHz, d₄-MeOH): δ 7.39-7.38 (2H, d), 7.34-7.29 (4H, m), 7.26-7.23 (1H, t), 6.98-6.96 (2H, d), 5.47-5.43 (1H, q), 4.26-4.22 (6H, m), 3.66-3.61 (1H, m), 1.63-1.61 (3H, d). LC/MS (Table 2, Method c) Rₜ: 0.97 min; m/z 312.2 (M+H)⁺. Chiral HPLC (Table 2, Method e) ee value: 95.4.

### Preparation #15: Synthesis of (S)-1-(4-(1-Phenylethoxy)benzyl)azetidine-3-carboxylic acid

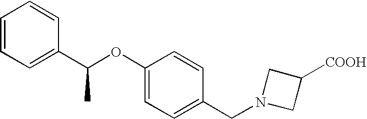

(S)-1-(4-(1-Phenylethoxy)benzyl)azetidine-3-carboxylic acid was separated by Prep-Chiral-HPLC using an OJ-H column and hexane:ethanol=75%:25% as eluent from 1-(4-(1-phenylethoxy)benzyl)azetidine-3-carboxylic acid (Example #10), using (R)-1-(4-(1-phenylethoxy)benzyl)azetidine-3-carboxylic acid to confirm the configuration. ¹H NMR (500 MHz, d₄-MeOH): δ 7.27-7.26 (2H, d), 7.22-7.21 (4H, m), 7.14-7.11 (1H, t), 6.86-6.84 (2H, d), 5.35-5.31 (1H, q), 4.16-4.12 (6H, m), 3.55-3.51 (1H, m), 1.51-1.50 (3H, d). LC/MS (Table 1, Method c) Rₜ: 0.92 min; m/z 312.2 (M+H)⁺. Chiral HPLC (Table 2, Method e) ee value: 96.1.

### Preparation #16: Synthesis of 5-Bromo-2-(3-(trifluoromethyl)benzyloxy)pyridine

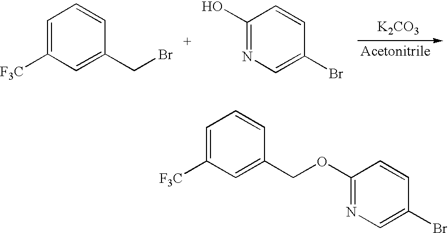

K₂CO₃ (2.07 g, 15 mmol) was added to a mixture of 3-(trifluoromethyl)benzyl bromide (1.18 g, 5 mmol) and 5-bromo-2-hydroxypyridine (0.87 g, 5 mmol) in acetonitrile (30 mL) and the mixture was stirred for about 18 h at room temperature. After concentrating, the residue was purified by silica-gel column chromatography (PE:EtOAc=4:1) to afford 5-bromo-2-(3-(trifluoromethyl)benzyloxy)pyridine as a white solid (1.32 g, yield 80%). ¹H NMR (500 MHz, CDCl₃): δ 7.60-7.59 (t, 1H), 7.57 (s, 1H), 7.50-7.49 (m, 2H), 7.41-7.36 (2H, m), 6.57 (d, 1H, J=9.5 Hz), 5.15 (s, 2H). LC/MS (Table 2, Method c) Rₜ: 0.75 min; m/z 332.0 (M+H)⁺.

### Preparation #17: Synthesis of 6-(3-(Trifluoromethyl)benzyloxy)nicotinonitrile

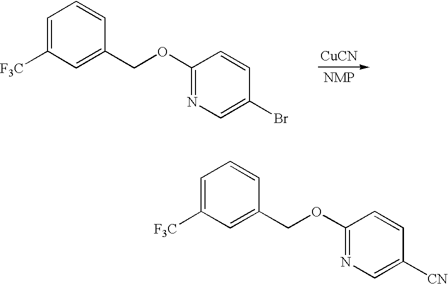

A mixture of 5-bromo-2-(3-(trifluoromethyl)benzyloxy)pyridine (Preparation #16) (1.3 g, 11.34 mmol) and CuCN (2.49 g, 37.84 mmol) in N-methyl-2-pyrrolidone (20 mL) was heated to about 160° C. for about 32 h. After cooling slightly, the mixture was washed with 5% aqueous NaCN and extracted with ether. Extracts were washed with 5% aqueous NaCN, water, brine, dried (Na₂SO₄) and filtered. After removal of solvent in vacuo, purification on silica-gel column chromatography (PE:EtOAc=10:1) afforded 6-(3-(trifluoromethyl)benzyloxy)nicotinonitrile as a white solid (1.3 g, yield 41%). LC/MS (Table 2, Method c) Rₜ: 1.12 min; m/z 297.1 (M+H)⁺.

### Preparation #18: Synthesis of 6-(3-(Trifluoromethyl)benzyloxy)nicotinic acid

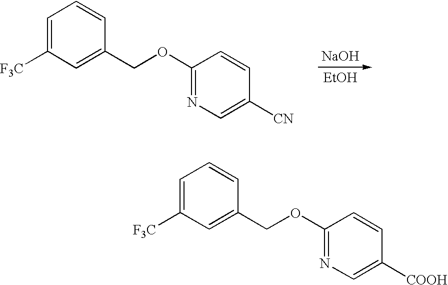

6-(3-(Trifluoromethyl)benzyloxy)nicotinonitrile (Preparation #17) (565 mg, 2.03 mmol) in 15 mL of ethanol and 11.7 mL of 10 M NaOH were refluxed for about 6.5 h. The mixture was cooled, poured onto water and acidified with 2 M HCl, and the precipitated crystals were filtered off under suction, washed with water and dried to give 6-(3-(trifluoromethyl)benzyloxy)nicotinic acid (0.3 g, yield 49%). LC/MS (Table 2, Method c) Rₜ: 0.53 min; m/z 298.1 (M+H)⁺.

### Preparation #19: Synthesis of (6-(3-(Trifluoromethyl)benzyloxy)pyridin-3-yl)methanol

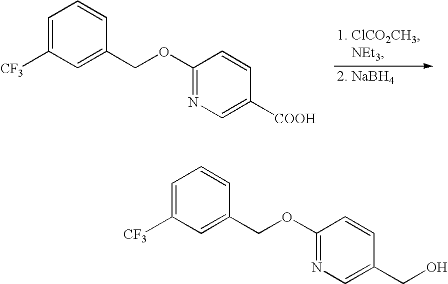

To a mixture of 6-(3-(trifluoromethyl)benzyloxy)nicotinic acid (Preparation #18) (0.3 g, 1.01 mmol) and Et₃N (105 mg, 1.04 mmol) in dry THF (15 mL) at about −10° C. was added dropwise a solution of methyl chloroformate (97 mg, 1.03 mmol) in THF. The mixture was stirred at about 10° C. for about 20 min and then warmed to about 0° C. NaBH₄ (111 mg, 2.93 mmol) was added followed by dropwise addition of CH₃OH (15 mL). Stirring was continued at about 0° C. for about 20 min and then the solution was allowed to warm to room temperature. 10% Critic acid was added and the mixture was concentrated in vacuo. The residue was extracted with EtOAc and the extract washed with water and brine, dried (Na₂SO₄), filtered and concentrated. Purification by silica-gel column chromatography (PE:EtOAc from 10:1 to 2:1) afforded (6-(3-(trifluoromethyl)benzyloxy)pyridin-3-yl)methanol as a yellow oil (127 mg, yield 43%). LC/MS (Table 2, Method c) Rₜ: 1.33 min; m/z 284.0 (M+H)⁺.

### Exemplification of General Procedure K

### Example #K.1

### Synthesis of 1-((6-(3-(trifluoromethyl)benzyloxy)pyridin-3-yl)methyl)azetidine-3-carboxylic acid

### Step A: Preparation #20: Synthesis of 6-(3-(Trifluoromethyl)benzyloxy)nicotinaldehyde

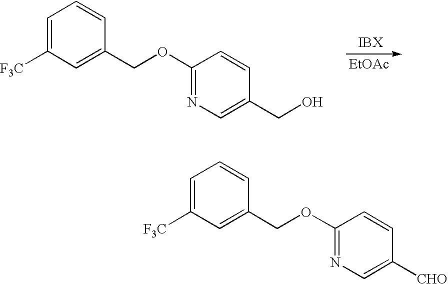

(6-(3-(Trifluoromethyl)benzyloxy)pyridin-3-yl)methanol (Preparation #19) (293 mg, 1.04 mmol) and IBX (1.01 g, 3.62 mmol) in EtOAc (15 mL) were refluxed at about 80° C. for about 2 h. The reaction mixture was cooled to room temperature and filtered. The filtrate was concentrated and the crude product purified by silica-gel column chromatography (PE:EtOAc=2:1) to afford 6-(3-(trifluoromethyl)benzyloxy)nicotinaldehyde as a white solid (111 mg, yield 38%). LC/MS (Table 2, Method c) Rₜ: 1.79 min; m/z 282.1 (M+H)⁺.

### Step B: Synthesis of 1-((6-(3-(trifluoromethyl)benzyloxy)pyridin-3-yl)methyl)azetidine-3-carboxylic acid

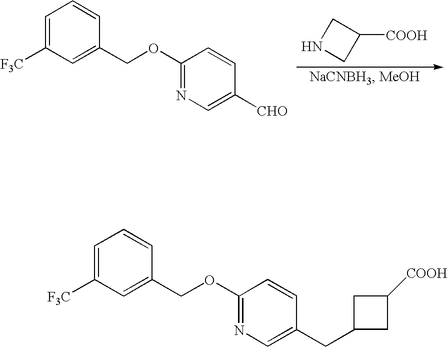

6-(3-(Trifluoromethyl)benzyloxy)nicotinaldehyde (Preparation #20) (111 mg, 0.4 mmol) was added to a stirred solution of azetidine-3-carboxylic acid (40 mg, 0.4 mmol) and HOAc (72 mg, 1.2 mmol) in CH₃OH (10 mL). The mixture was heated to about 40° C. After about 15 min, NaCNBH₃ (74 mg, 1.2 mmol) was added in a single portion and stirred at about 40° C. overnight. Purification by Prep-HPLC afforded 1-O-(3-(trifluoromethyl)benzyloxy)pyridin-3-yl)methyl)azetidine-3-carboxylic acid (68.9 mg, yield 47%). ¹H NMR (500 MHz, d₄-MeOH): δ 7.88 (1H, d), 7.57 (1H, s), 7.53-7.50 (2H, m), 7.48-7.44 (2H, m), 6.53-6.51 (1H, d), 5.17 (2H, s), 4.01-3.94 (6H, m), 3.28-3.25 (1H, m). LC/MS (Table 2, Method c) Rₜ: 1.31 min; m/z 367.1 (M+H)⁺.

Compounds in Table K were prepared using the same procedure as for 1-((6-(3-(trifluoromethyl)benzyloxy)pyridin-3-yl)methyl)azetidine-3-carboxylic acid, Example #K.1.

TABLE K

| | | | | LC/ | | | |
|---|---|---|---|---|---|---|---|
| | | Starting | | MS | Ob- | M + 1 | |
| Ex. | Starting | bromo | | Rₜ | served | or | |
| # | bromide | pyridine | Structure | (min) | mass | M − 1 | NMR |
| K.2 | 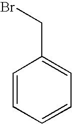 | 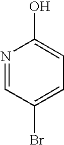 | 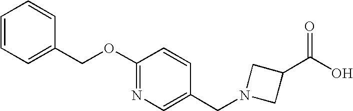 | 1.36 | 299 | M + 1 | ¹H NMR (500 MHz, d₄-MeOH): δ 7.94 (1H, s), 7.58-7.56 (1H, m), 7.38-7.31 (5H, m), 6.64-6.62 (1H, d), 5.22 (2H, s), 4.11-4.08 (6H, m), 3.41-3.35 (1H, m) |
| K.3 | 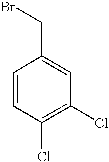 | 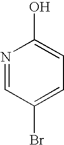 | 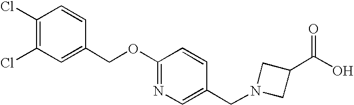 | 1.33 | 367.1 | M + 1 | ¹H NMR (500 MHz, d₄-MeOH): δ 7.95 (1H, d), 7.59-7.57 (1H, q), 7.55-7.54 (1H, d), 7.53-7.51 (1H, d), 7.30-7.28 (1H, q), 6.63-6.62 (1H, d), 5.18 (2H, s), 4.10-4.01 (6H, m), 3.38-3.34 (1H, m) |

### Exemplification of General Procedure L

### Example #L.1

### Exemplification of General Procedure L: Synthesis of 1-(4-(2-Phenylacetyl)benzyl)pyrrolidine-3-carboxylic acid

### Step A: Preparation #21: Synthesis of 4-(2-Phenylacetyl)benzaldehyde

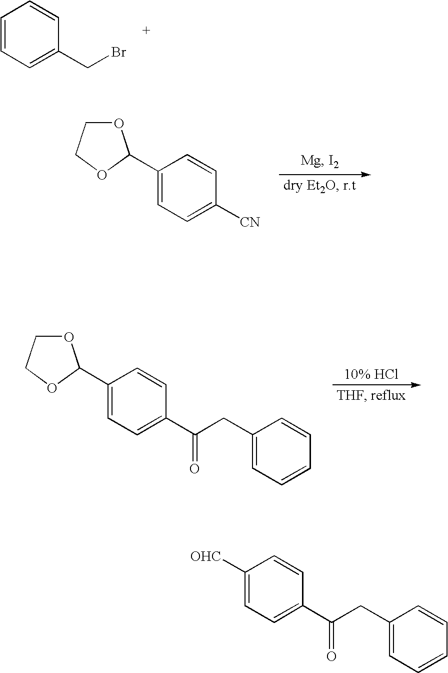

Under N₂, to magnesium turnings (792 mg, 32.94 mmol) and iodine (7 mg) in Et₂O (50 mL), was added a solution of benzyl bromide (3.76 g, 21.99 mmol) at room temperature. After stirring for about 1 h, the mixture was cooled to about 0-15° C. A solution of 4-formylbenzonitrile (2.0 g, 11.43 mmol) in Et₂O (15 mL) was added dropwise, then the solution was refluxed for about 1 h. The solution was cooled to room temperature and treated with ice-water. Aqueous 5 M HCl was added, the organic phase was separated and the aqueous phase was extracted with EtOAc. The combined organic phase was washed with saturated NaHSO₃ and saturated NaHCO₃, dried (Na₂SO₄), filtered and concentrated in vacuo to afford the crude 1-(4-(1,3-dioxolan-2-yl)phenyl)-2-phenylethanone. The crude 1-(4-(1,3-dioxolan-2-yl)phenyl)-2-phenylethanone was dissolved in THF (50 mL) and 10% HCl (50 mL) was added to the solution. The reaction mixture was refluxed for about 16 h. The reaction mixture was cooled to room temperature and EtOAc was added to extract the compound. The mixture was dried (Na₂SO₄), filtered and the solvent removed. Purification by silica-gel column chromatography (PE/EtOAc from 25:1 to 10:1) afforded 4-(2-phenylacetyl)benzaldehyde as a white solid (1.1 g, yield 43%). LC/MS (Table 2, Method c) Rₜ: 1.57 min; m/z 225.1 (M+H)⁺.

### Step B: Synthesis of 1-(4-(2-Phenylacetyl)benzyl)pyrrolidine-3-carboxylic acid

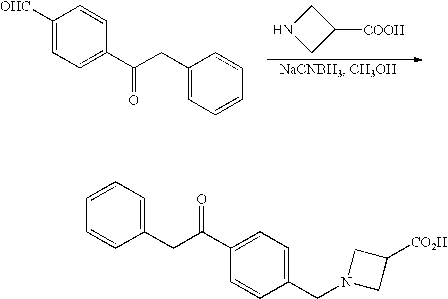

4-(2-Phenylacetyl)benzaldehyde (Preparation #21) (133 mg, 0.59 mmol) was added to a stirred solution of azetidine-3-carboxylic acid (60 mg, 0.59 mmol) and HOAc (107 mg, 1.78 mmol) in 10 mL CH₃OH. The mixture was heated to about 40° C. After about 15 min, NaCNBH₃ (110 mg, 1.78 mmol) was added in a single portion and stirred at about 40° C. overnight. After acidifying with 1 M HCl, purification by HPLC afforded 1-(4-(2-phenylacetyl)benzyl)pyrrolidine-3-carboxylic acid (38.7, yield 21%). ¹H NMR (500 MHz, δd₄-MeOH): δ 8.15 (d, 2H, J=7.5 Hz), 7.61 (d, 2H, J=8.0 Hz), 7.33-7.23 (m, 5H), 4.50 (s, 2H), 4.39 (s, 2H), 4.36 (d, 4H, J=7.5 Hz), 3.75-3.68 (m, 1H). LC/MS (Table 2, Method c) Rₜ: 1.23 min; m/z 310.2 (M+H)⁺.

Compounds in Table L were prepared using the same procedure as for 1-(4-(2-phenylacetyl)benzyl)pyrrolidine-3-carboxylic acid, Example #L.1.

TABLE L

| | | | | LC/ | | | |
|---|---|---|---|---|---|---|---|
| | | | | MS | Ob- | M + 1 | |
| Ex. | Starting | Starting | | Rₜ | served | or | |
| # | amine | bromide | Product | (min) | mass | M − 1 | NMR |
| L.2 | 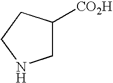 | 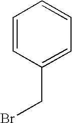 | 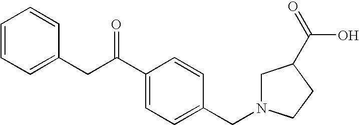 | 1.24 | 324.2 | M + 1 | ¹H NMR (500 MHz, d₄-MeOH): δ 8.14 (d, 2H, J = 8.0 Hz), 7.65 (d, 2H, J = 8.5 Hz), 7.31- 7.21 (m, 5H), 4.48 (s, 2H), 4.36 (s, 2H), 3.65-3.57 (m, 2H), 3.44-3.39 (m, 3H), 2.43- 2.33 (m, 2H). |
| L.3 | 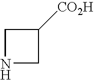 | 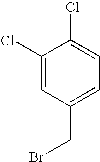 | 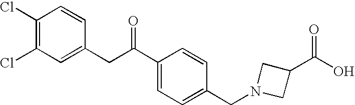 | 1.39 | 378.1 | M + 1 | ¹H NMR (500 MHz, d₄-MeOH): δ 8.05-8.03 (2H, d), 7.53-7.51 (2H, d), 7.37-7.36 (2H, d), 7.10- 7.08 (1H, m), 4.40 (2H, s), 4.31 (2H, s), 4.27-4.22 (4H, 3.62-3.59 (1H, m) |
| L.4 | 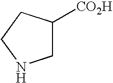 | 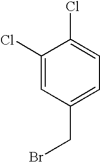 | 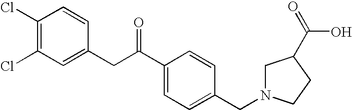 | 1.4 | 392.1 | M + 1 | ¹H NMR (500 MHz, d₄-MeOH): δ 8.14-8.13 (2H, d), 7.66-7.65 (2H, d), 7.48-7.47 (2H, m), 7.23-7.21 (1H, q), 4.36-4.28 (2H, q), 3.42-3.41 (1H, m), 3.30- 3.11 (4H, m), 2.32-2.24 (2H, m) |

### Exemplification of General Procedure M

### Example #M.1

### Synthesis of 1-(4-Hexanoylbenzyliazetidine-3-carboxylic acid

### Step A: Preparation #22: Synthesis of 4-Hexanoylbenzaldehyde

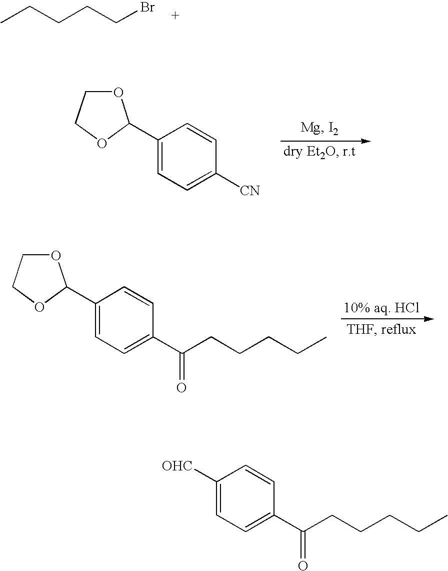

To magnesium turnings (0.48 g, 20.0 mmol) and iodine (7 mg) in Et₂O (50 mL), was added a solution of 1-bromopentane (2.00 g, 13.3 mmol) at room temperature under a nitrogen atmosphere. After stirring for about 1 h, the mixture was cooled to about 0-15° C. 4-(1,3-dioxolan-2-yl)benzonitrile (1.75 g, 10.0 mmol) in Et₂O (15 mL) was added dropwise, then the solution was refluxed for about 1 h. The solution was cooled to room temperature and treated with ice-water. Aqueous 5 M HCl was added, the organic phase was separated and the aqueous phase was extracted with EtOAc. The combined organic phase was washed with saturated NaHSO₃ and saturated NaHCO₃, dried (Na₂SO₄) and concentrated in vacuo to get the crude 1-(4-(1,3-dioxolan-2-yl)phenyl)hexan-1-one. The crude 1-(4-(1,3-dioxolan-2-yl)phenyl)hexan-1-one was dissolved in THF (50 mL) and 10% HCl (50 mL) was added to the solution. The reaction mixture was refluxed for about 16 h. The reaction mixture was cooled to room temperature, and then EtOAc was added to extract the compound. After drying with Na₂SO₄ and removing the solvent, the crude compound was purified by silica-gel column chromatography (PE/EA from 25:1 to 10:1) to afford 4-hexanoylbenzaldehyde as a white solid (1.5 g, yield 73%). LC/MS (Table 2, Method c) Rₜ: 1.71 min; m/z 205.2 (M+H)⁺.

### Step B: Synthesis of 1-(4-Hexanoylbenzyl)azetidine-3-carboxylic acid

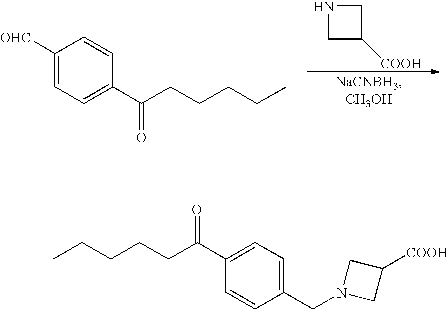

4-Hexanoylbenzaldehyde (Preparation #22) (120 mg, 0.59 mmol) was added to a stirred solution of azetidine-3-carboxylic acid (60 mg, 0.59 mmol) and HOAc (107 mg, 1.78 mmol) in 10 mL CH₃OH. The mixture was heated to about 40° C. After about 15 min, NaCNBH₃ (110 mg, 1.78 mmol) was added in a single portion and the mixture was stirred at about 40° C. overnight. After acidifying with 1 M HCl, purification by Prep-HPLC afforded 1-(4-hexanoylbenzyl)azetidine-3-carboxylic acid (14.5 mg, yield 8.5%): ¹H NMR (500 MHz, d₄-MeOH): δ 8.08 (d, 2H, J=8.0 Hz), 7.60 (d, 2H, J=8.5 Hz), 4.39 (s, 2H), 4.19-4.15 (m, 4H), 3.48-3.38 (m, 1H) 306 (t, 2H, J=7.3 Hz), 1.77-1.71 (m, 2H), 1.41-1.39 (m, 4H), 0.96 (t, 3H, J=7.1 Hz). LC/MS (Table 2, Method c) Rₜ: 1.32 min, m/z 290.2 (M+H)⁺.

Compounds in Table M were prepared using the same procedure as for 1-(4-hexanoylbenzyl)azetidine-3-carboxylic acid, Example #M.1.

TABLE M

| | | | | LC/MS | Ob- | M + 1 | |
|---|---|---|---|---|---|---|---|
| Ex. | Starting | Starting | | Rₜ | served | or | |
| # | amine | bromide | Product | (min) | mass | M − 1 | NMR |
| M.2 | 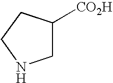 | 1-Bromo pentane | 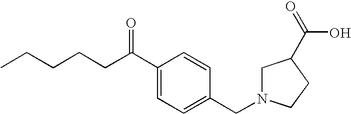 | 1.32 | 304.3 | M + 1 | ¹H NMR (300 MHz, d₄-MeOH): δ 8.06-8.04 (2H, d), 7.66-7.64 (2H, d), 4.38-4.31 (2H, q), 3.48-3.46 (1H, m), 3.33-3.31 (2H, m), 3.25-3.08 (2H, m), 3.06-3.03 (2H, t), 2.32-2.26 (2H, m), 1.74- 1.70 (2H, m), 1.40-1.37 (4H, m), 0.96-0.93 (3H, t) |
| M.3 | 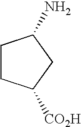 | 1-Bromo pentane | 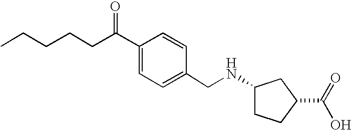 | 1.34 | 318.3 | M + 1 | ¹H NMR (500 MHz, d₄-MeOH): δ 8.09 (d, 2H, J = 8 Hz), 7.65 (d, 2H, J = 8 Hz), 4.36-4.29 (m, 2H), 3.75-3.70 (m, 1H), 3.05 (m, 3H), 2.46-2.43 (m, 1H), 2.25-2.20 (m, 1H), 2.09- 2.08 (m, 3H), 1.96- 1.88 (m, 1H), 1.76-1.70 (m, 2H), 1.42-1.30 (m, 5H), 0.94 (t, 3H, J = 7 Hz). |
| M.4 | 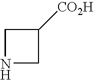 | 1-Bromo hexane | 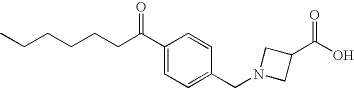 | 1.39 | 304.3 | M + 1 | ¹H NMR (500 MHz, d₄-MeOH): δ 8.08-8.06 (2H, d), 7.59-7.58 (2H, d), 4.38 (2H, s), 4.18-4.12 (4H, m), 3.45-3.40 (1H, m), 3.06- 3.03 (2H, t), 1.75-1.70 (2H, m), 1.42-1.31 (6H, m), 0.94-0.92 (3H, t) |
| M.5 | 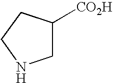 | 1-Bromo hexane | 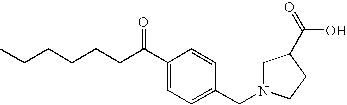 | 1.4 | 318.3 | M + 1 | ¹H NMR (500 MHz, d₄-MeOH): δ 8.04-8.02 (2H, d), 7.67-7.65 (2H, d), 4.39-4.33 (2H, q), 3.49 (1H, s), 3.37-3.32 (2H, m), 3.27-3.23 (2H, m), 3.04-3.01 (2H, t), 2.37-2.23 (2H, m), 1.73-1.67 (2H, m), 1.44- 1.37 (2H, m), 1.36-1.31 (4H, m), 0.93-0.90 (3H, t) |
| M.6 | 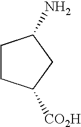 | 1-Bromo hexane | 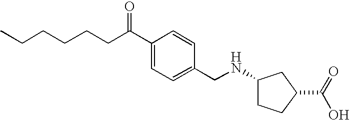 | 1.41 | 332.3 | M + 1 | ¹H NMR (500 MHz, d₄-MeOH): δ 8.08-8.07 (2H, d), 7.66-7.64 (2H, d), 4.35-4.32 (1H, d), 4.25-4.22 (1H, d), 3.73 (1H, m), 3.06-3.03 (2H, m), 2.22-1.98 (6H, m), 1.73-1.71 (2H, m), 1.42-1.31 (8H, m), 0.94- 0.92 (3H, m) |

### Example #11

### Synthesis of 1-(4-(3,3-Dimethylbut-1-ynyl)benzyl)azetidine-3-carboxylic acid

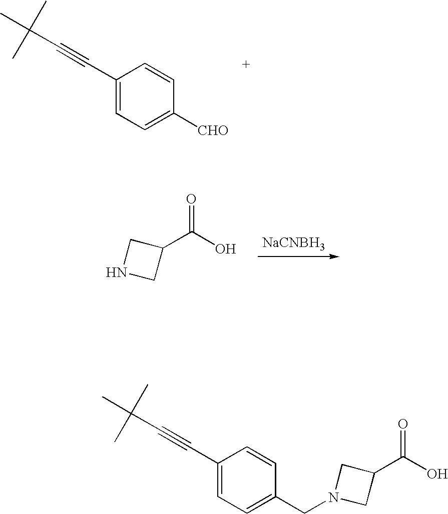

A solution of 4-(3,3-dimethylbut-1-ynyl)benzaldehyde (24 mg, 0.13 mmol) (ChemPacific) dissolved in DCM/MeOH (1.0 mL) was added to a 20 mL vial, followed by azetidine-3-carboxylic acid (9 mg, 0.15 mmol). Then, HOAc (22 μL, 0.4 mmol) was added. The vial was capped and stirred at about 50° C. for about 2 h, followed by the addition of 285 mg of MP-cyanoborohydride resin (Biotage, 5 eq). The reaction was then heated with shaking overnight at about 50° C. The reaction was checked by LC/MS and concentrated to dryness. The residue was dissolved in 1:1 DMSO/MeOH and purified by reverse phase HPLC to afford 1-(4-(3,3-dimethylbut-1-ynyl)benzyl)azetidine-3-carboxylic acid. LC/MS (Table 2, Method c) Rₜ: 1.42 min; m/z 272 (M+H)⁺.

### Preparation #23: Synthesis of 2-Fluoro-4-(3-(trifluoromethyl)benzyloxy)benzonitrile

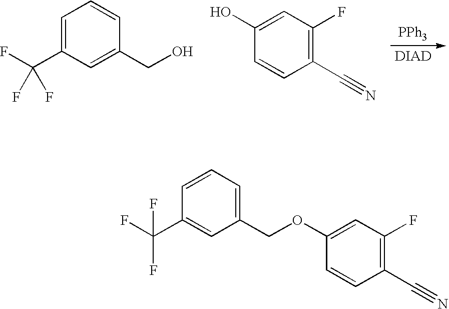

In a 1 L round-bottomed flask, DIAD (14.31 mL, 72.7 mmol) and triphenylphosphine (19.07 g, 72.7 mmol) in THF (200 mL) were stirred for about 5 min under nitrogen and cooled to about 0° C. 2-Fluoro-4-hydroxybenzonitrile (6.65 g, 48.5 mmol) was added to give a dark orange solution. The mixture was stirred about an additional 5 min and then 3-(trifluoromethyl)benzyl alcohol (7.26 mL, 53.3 mmol) in THF (50 mL) was added. The mixture was stirred overnight at ambient temperature then evaporated to dryness. The solid was purified on a Combiflash Companion XL system using a 330 g Redi-Sep silica gel column using the following gradient: A: Heptane; B: Ethyl acetate; 10 to 100% B over 7 column volumes. NMR indicated the presence of triphenyl phosphine oxide and reduced DIAD. The residue was triturated with light petroleum ether (250 mL) for 1 hour, filtered and the solid dried under vacuum overnight. This gave 2-fluoro-4-(3-(trifluoromethyl)benzyloxy)benzonitrile (9.31 g, 31.2 mmol, 99%). ¹H NMR (400 MHz, DMSO-d₆): δ 7.91-7.83 (m, 2H), 7.78 (d, J=7.7, 1H), 7.74 (d, J=7.8, 1H), 7.66 (t, J=7.7, 1H), 7.30 (dd, J=2.4, 11.9, 1H), 7.09 (dd, J=2.4, 8.8, 1H), 5.34 (s, 2H). (Table 2, Method b) Rₜ=1.60 min; MS m/z: 294.04 (M−H)⁻.

### Preparation #24: Synthesis of 2-Fluoro-4-(3-(trifluoromethyl)benzyloxy)benzaldehyde

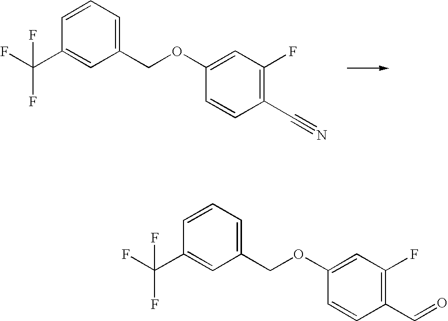

In a heat dried 500 mL round-bottomed flask, 2-fluoro-4-(3-(trifluoromethyl)benzyloxy)benzonitrile (9.00 g, 30.5 mmol) in toluene (125 mL) was added to give a colorless solution. The solution was cooled to about 0° C. in an ice bath. The 1.0 M diisobutylaluminum hydride (61.0 mL, 61.0 mmol) in hexane was added dropwise via addition funnel to the solution, keeping the temperature <8° C. After addition was complete, the solution was stirred at about 0° C. for about an additional hour then stirred at ambient temperature overnight. Chloroform (125 mL) was added followed by 10% aqueous hydrochloric acid (325 mL). Stirring for about 1 h at ambient temperature afforded an emulsion that was allowed to settle over the weekend. The layers were separated and the aqueous layer aqueous layer with chloroform and washed combined organic layers with water (200 mL) and brine (200 mL). Dried over MgSO₄, filtered and removed solvent in vacuo to afford 2-fluoro-4-(3-(trifluoromethyl)benzyloxy)benzaldehyde (8.02 g, 26.6 mmol, 87% yield). ¹H NMR (400 MHz, DMSO-d₆) δ 10.08 (s, 1H), 7.85 (s, 1H), 7.82 (t, J=8.5, 1H), 7.79 (d, J=7.7, 1H), 7.74 (d, J=8.0, 1H), 7.67 (t, J=7.7, 1H), 7.14 (dd, J=2.3, 12.9, 1H), 7.06 (dd, J=2.3, 8.7, 1H), 5.36 (s, 2H). (Table 2, Method b) LC/MS Rₜ=2.81 min; MS m/z: did not ionize.

### General Procedure N: Synthesis of Compounds in Table N

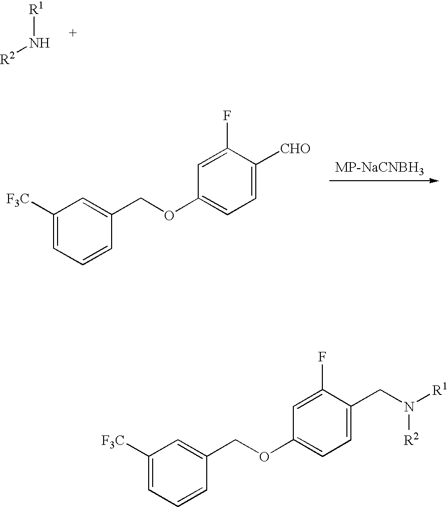

Compounds in Table N were produced as part of a one dimensional array with the only variant being the aldehyde monomer which is given in Table N. The amines were purchased pre-weighed from the Sigma Aldrich Custom Packaged Reagent service.

In a 20 mL vial a solution of 2-fluoro-4-(3-(trifluoromethyl)benzyloxy)benzaldehyde (Preparation #24) (25 mg, 0.08 mmol) dissolved in DCM/MeOH (1.0 mL) was added, followed by amine monomer (0.1 mmol) dissolved in DCM/MeOH (0.3 mL). Then, to the solution was added HOAc (24 μL, 0.4 mmol) neat. The vial was capped and stirred at about 50° C. for about 2 hours, followed by the addition of 186 mg of MPNaCNBH₃ resin (5 eq.; subst. 2.25 mmol/g). The reaction was then heated with shaking overnight at about 50° C. The reaction was checked by LC/MS and concentrated to dryness. The residue was dissolved in 1:1 DMSO/MeOH and purified by reverse phase HPLC. Product was characterized by MS and LC/MS (Table 2, Method a).

TABLE N

| | | | LC/MS | Ob- | M + 1 |
|---|---|---|---|---|---|
| | | | Rₜ | served | or |
| Ex. | Starting amine | Product | (min) | mass | M − 1 |
| N.1 | 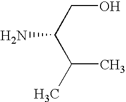 | 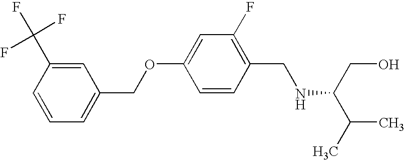 | 1.41 | 386 | M + 1 |
| N.2 | 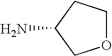 | 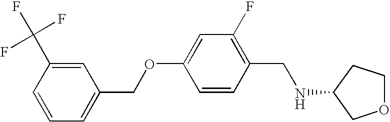 | 1.37 | 370 | M + 1 |
| N.3 | 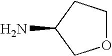 | 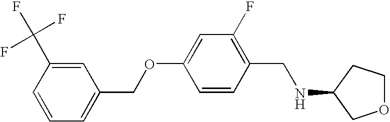 | 1.35 | 370 | M + 1 |
| N.4 | 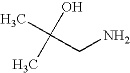 | 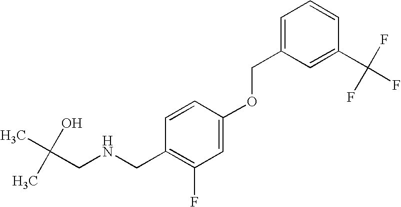 | 1.36 | 372 | M + 1 |
| N.5 | 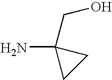 | 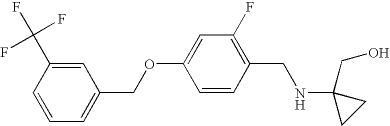 | 1.37 | 370 | M + 1 |
| N.6 | 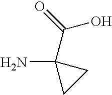 | 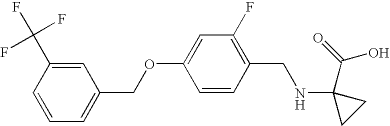 | 1.37 | 384 | M + 1 |
| N.7 | 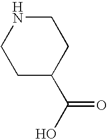 | 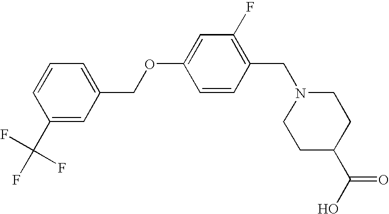 | 1.38 | 412 | M + 1 |
| N.8 | 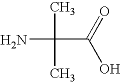 | 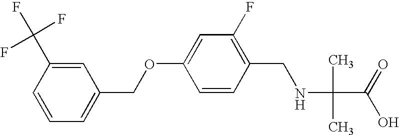 | 1.36 | 386 | M + 1 |
| N.9 | 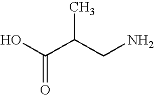 | 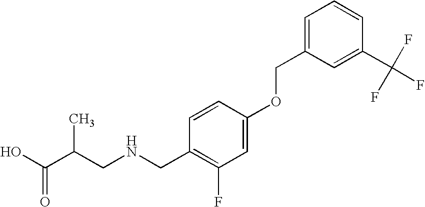 | 1.38 | 386 | M + 1 |
| N.10 | 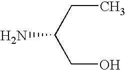 | 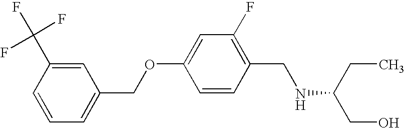 | 1.38 | 372 | M + 1 |
| N.11 | 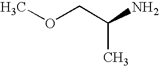 | 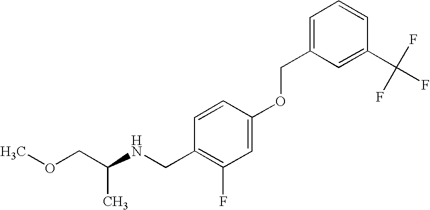 | 1.41 | 372 | M + 1 |
| N.12 | 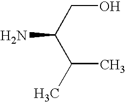 | 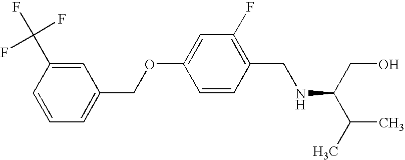 | 1.41 | 386 | M + 1 |
| N.13 | 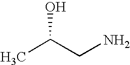 | 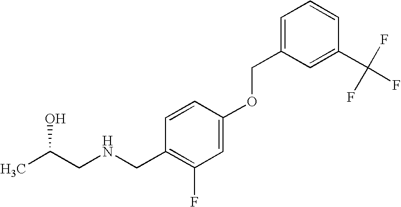 | 1.35 | 358 | M + 1 |
| N.14 | 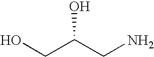 | 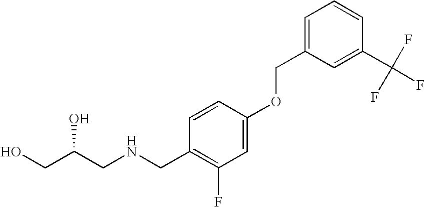 | 1.26 | 374 | M + 1 |
| N.15 | 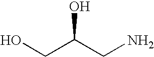 | 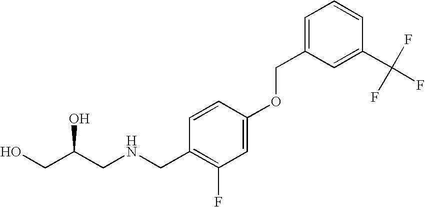 | 1.27 | 374 | M + 1 |
| N.16 | 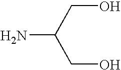 | 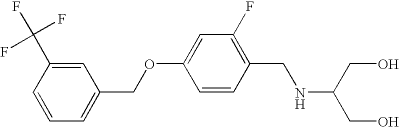 | 1.28 | 374 | M + 1 |
| N.17 | 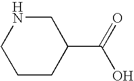 | 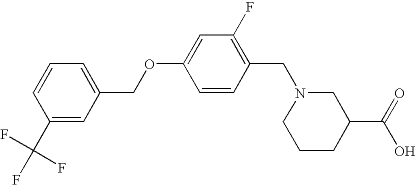 | 1.38 | 412 | M + 1 |
| N.18 | 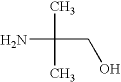 | 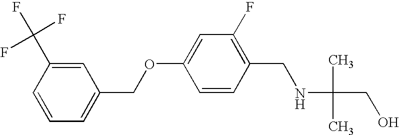 | 1.35 | 372 | M + 1 |
| N.19 | 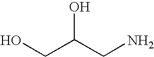 | 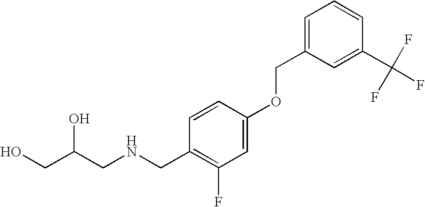 | 1.28 | 374 | M + 1 |
| N.20 | 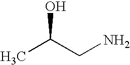 | 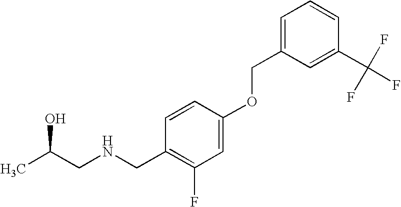 | 1.34 | 358 | M + 1 |
| N.21 | 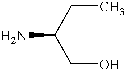 | 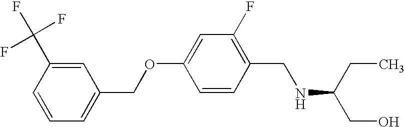 | 1.37 | 372 | M + 1 |
| N.22 | 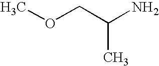 | 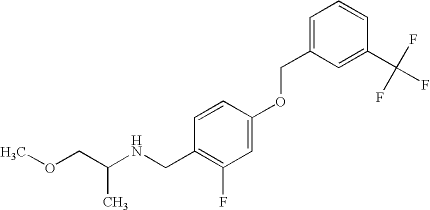 | 1.39 | 372 | M + 1 |
| N.23 | 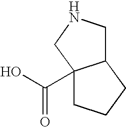 | 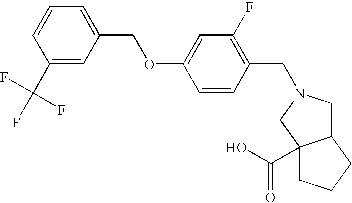 | 1.45 | 438 | M + 1 |
| N.24 | 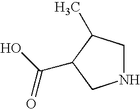 | 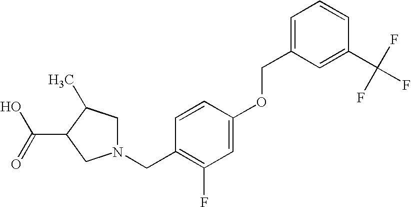 | 1.41 | 412 | M + 1 |

### Example #12

### Synthesis of 3-(4-(Benzyloxy)phenylamino)cyclopentanecarboxylic acid

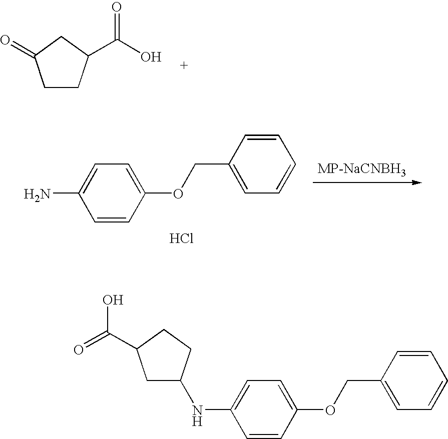

To a stirring solution of 3-oxocyclopentanecarboxylic acid (250 mg, 1.951 mmol) and 4-benzyloxyaniline hydrochloride (506 mg, 2.146 mmol) was added MP-Cyanoborohydride (7160 mg, 7.80 mmol) and HOAc (0.447 ml, 7.80 mmol). The slurry was stirred at room temperature overnight (about 16 h). The suspension was filtered and the resin washed with MeOH (2×60 mL). The filtrate was concentrated in vacuo to provide the crude product. The crude product was added to a silica gel column and was eluted with MeOH/DCM (0%-10%, 30 min then 10% 10 min). The fractions containing the correct molecular weight by LC/MS and/or TLC were combined and concentrated to provide the desired product as a white powder. ¹HMR and LC/MS indicate product is contaminated with ˜5-10% impurity(ies). The material was again added to a silica gel column and was eluted with MeOH/DCM (0%-10%, 30 min). The fractions containing the correct MW by LC/MS and/or TLC were combined and concentrated to provide 3-(4-(benzyloxy)phenylamino)cyclopentanecarboxylic acid (170.4 mg, 0.547 mmol, 28.0% yield) as a white powder. ¹H NMR (DMSO-d₆) δ 12.02 (br s, 1H), 7.38 (m, 4H), 7.30 (t, J=7.75, 1H), 6.77 (d, J=8.89 Hz, 2H), 6.49 (d, J=8.89 Hz, 2H), 5.12 (br s, 1H), 4.95 (s, 2H), 3.64 (dt, J=13.72 Hz, 6.91 Hz, 1H), 2.71 (dt, J=16.92 Hz, 8.46 Hz, 1H), 2.54 (ddd, J=12.88 Hz, 7.51 Hz, 7.37 Hz, 1H), 1.91 (m, 1H), 1.82 (m, 1H), 1.55 (m, 2H), 1.45 (m, 1H). LC/MS (Table 2, Method f) Rₜ=1.26 min; MS m/z 312.2 (M+H)⁺

### Example #13

### Synthesis of 1-(1-(4-(benzyloxy)phenyl)ethyl)azetidine-3-carboxylic acid

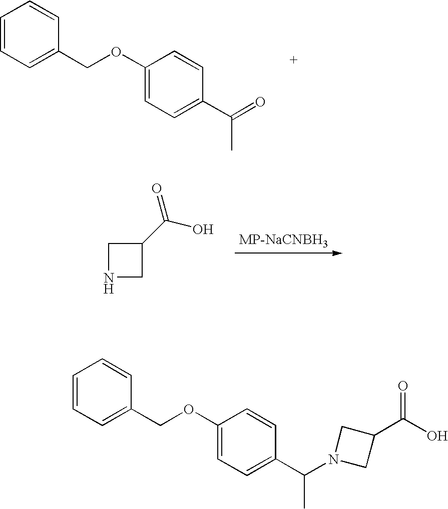

MP-Cyanoborohydride (12.68 g, 29.7 mmol) (Biotage), 1-(4-(benzyloxy)phenyl)ethanone (3.36 g, 14.84 mmol) and azetidine-3-carboxylic acid (1.5 g, 14.84 mmol) in MeOH (60 mL) and HOAc (12 drops) was stirred at ambient temperature in a 20 mL scintillation vial for about 3 days. The mixture was filtered and the resin washed copiously with DCM. Analysis by LC/MS showed about 20% conversion to the desired product, with about 80% of the starting ketone remaining. The combined organics were evaporated to dryness and submitted for purification by reversed phase HPLC. The combined fractions were evaporated to dryness, dried in vacuo at about 60° C. for about 24 h to afford 1-(1-(4-(benzyloxy)phenyl)ethyl)azetidine-3-carboxylic acid (121 mg, 0.365 mmol, 2.462% yield) as an off-white solid. ¹H NMR (400 MHz, DMSO-d₆) δ 7.49-7.26 (5H, m), 7.17 (2H, d, J=8.4 Hz), 6.91 (2H, d, J=8.5 Hz), 5.04 (2H, s), 3.36 (1H, d, J=9.9 Hz), 3.19 (1H, q, J=6.2 Hz), 3.12-2.94 (4H, m), 1.04 (3H, d, J=6.4 Hz). LC/MS (Table 2, Method b) Rₜ: 1.70 min; m/z 312 (M+H)⁺.

### Example #14

### Synthesis of ethyl 4-(4-(benzyloxy)phenoxy)cyclohexanecarboxylate

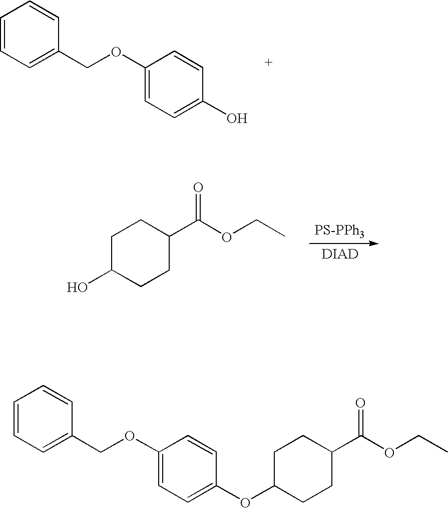

Triphenylphosphine (polymer bound, 3 mmol/g, 4.99 g, 14.98 mmol) was treated with DIAD (0.971 ml, 4.99 mmol) at about 0° C. in dry THF (30 mL). The mixture was stirred for about 1 h then 4-(benzyloxy)phenol (1.0 g, 4.99 mmol) and ethyl 4-hydroxycyclohexanecarboxylate (0.804 ml, 4.99 mmol) were added. The mixture was warmed to room temperature and then stirred overnight. The residue was evaporated to dryness and subjected to purification by reversed phase HPLC. The combined fractions were evaporated to dryness, dried in vacuo at about 60° C. for about 24 h to afford ethyl 4-(4-(benzyloxy)phenoxy)cyclohexanecarboxylate (501 mg, 1.413 mmol, 28.3% yield) as a pale green oil. ¹H NMR (400 MHz, CDCl₃): δ 7.51-7.31 (5H, m), 6.98-6.85 (4H, m), 5.04 (2H, d, J=4.8 Hz), 4.19 (2H, tt, J=13.3 Hz, 6.6 Hz), 4.14-4.06 (1H, m), 2.49-2.31 (1H, m), 2.26-1.96 (4H, m), 1.83-1.42 (4H, m), 1.34-1.25 (3H, m). LC/MS (Table 2, Method b) Rₜ: 3.17 min; m/z 353 (M+H)⁻.

### General Procedure O: Synthesis of Compounds in Table O

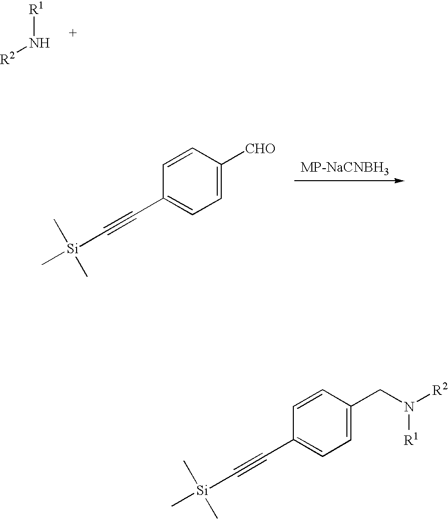

Compounds in Table O were produced as part of a one dimensional array with the only variant being the aldehyde monomer which is given in Table O. The amines were purchased pre-weighed from the Sigma Aldrich Custom Packaged Reagent service.

In a 20 mL vial a solution of 4-(trimethylsilyl)ethynylbenzaldehyde (1.2 eq) dissolved in MeOH:DCM (1.5 mL) was added, followed by the addition of amine core (20 mg, 1 eq.) dissolved in MeOH:DCM (1.0 mL), and HOAc (3 eq.). The mixture was shaken at about 50° C. for about 2 h and MP-cyanoborohydride resin (5 eq.) (Biotage) was added. This reaction mixture was allowed to stir overnight at about 50° C. The reaction was checked by LC/MS and concentrated to dryness. The residue was dissolved in 1:1 DMSO/MeOH and purified by reverse phase HPLC. Product was characterized by MS and LC/MS (Table 2, Method a).

TABLE O

| | | | LC/MS | Ob- | M + 1 |
|---|---|---|---|---|---|
| Ex. | | | Rₜ | served | or |
| # | Starting amine | Structure | (min) | mass | M − 1 |
| O.1 | 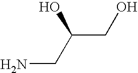 | 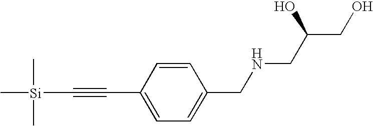 | 1.34 | 278 | M + 1 |
| O.2 | 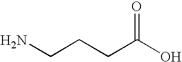 | 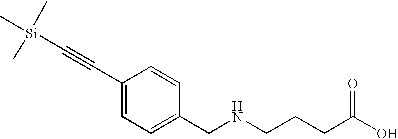 | 1.42 | 290 | M + 1 |
| O.3 | 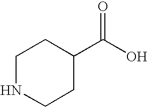 | 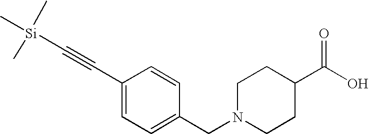 | 1.45 | 316 | M + 1 |
| O.4 | 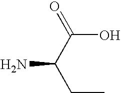 | 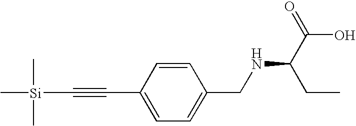 | 1.44 | 290 | M + 1 |
| O.5 | 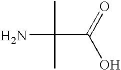 | 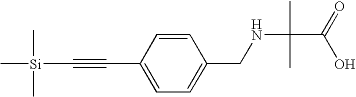 | 1.43 | 290 | M + 1 |
| O.6 | 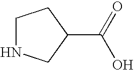 | 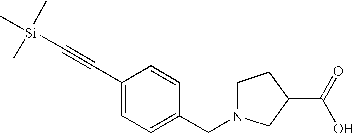 | 1.55 | 302 | M + 1 |
| O.7 | 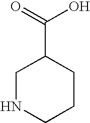 | 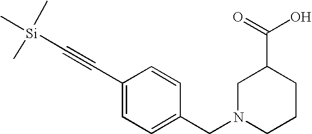 | 1.46 | 316 | M + 1 |
| O.8 | 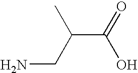 | 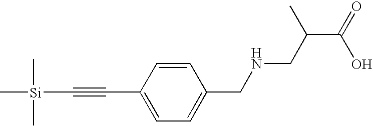 | 1.54 | 290 | M + 1 |
| O.9 | 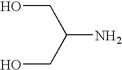 | 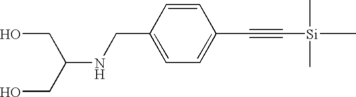 | 1.34 | 278 | M + 1 |
| O.10 | 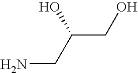 | 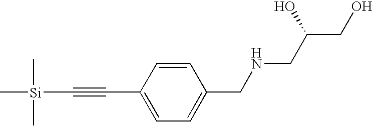 | 1.46 | 278 | M + 1 |
| O.11 | 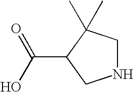 | 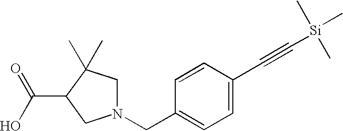 | 1.63 | 330 | M + 1 |
| O.12 | 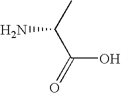 | 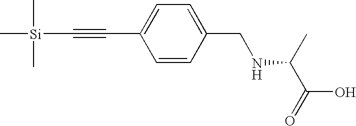 | 1.4 | 276 | M + 1 |
| O.13 | 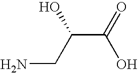 | 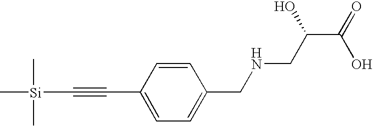 | 1.38 | 292 | M + 1 |
| O.14 | 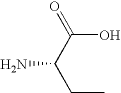 | 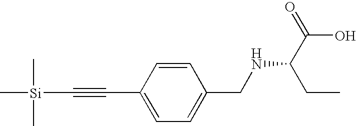 | 1.5 | 290 | M + 1 |
| O.15 | 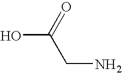 | 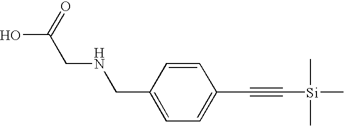 | 1.4 | 262 | M + 1 |
| O.16 | 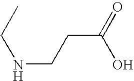 | 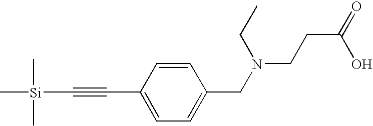 | 1.55 | 304 | M + 1 |
| O.17 | 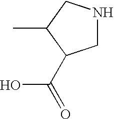 | 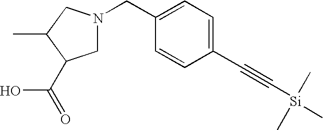 | 1.47 | 316 | M + 1 |
| O.18 | 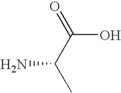 | 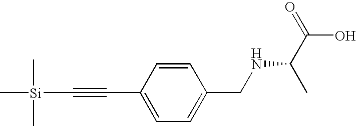 | 1.41 | 276 | M + 1 |

### General Procedure P: Synthesis of Compounds in Table P

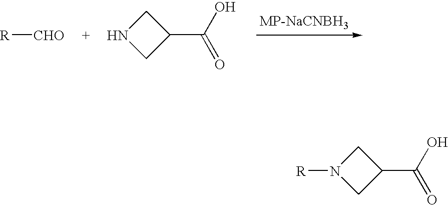

Compounds in Table P were produced as part of a one dimensional array with the only variant being the aldehyde monomer which is given in Table P. The aldehydes were purchased pre-weighed from the Sigma Aldrich Custom Packaged Reagent service.

In a 20 mL vial a solution of the aldehyde monomer (1.2 eq) dissolved in DCM (1.5 mL) was added, followed by the addition of azetidine-3-carboxylic acid (25 mg, 1 eq.) dissolved in DCM (1.0 mL), acetic acid (3 eq.) and MP-cyanoborohydride resin (3 eq.) (Biotage). The mixture was shaken at room temperature for about 4 to 5 hours. The reaction was checked by LC/MS and concentrated to dryness. The residue was dissolved in 1:1 DMSO/MeOH and purified by reverse phase HPLC. Product was characterized by MS and LC/MS (Table 2, Method a).

TABLE P

| | | | LC/MS | Ob- | M + 1 |
|---|---|---|---|---|---|
| Ex. | | | Rₜ | served | or |
| # | Starting aldehyde | Structure | (min) | mass | M − 1 |
| P.1 | 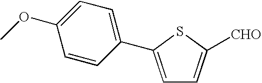 | 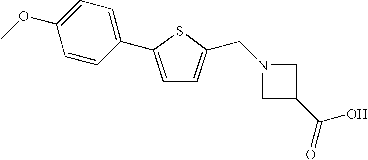 | 1.21 | 304 | M + 1 |
| P.2 | 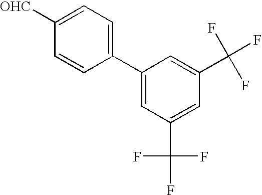 | 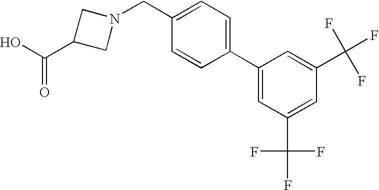 | 1.52 | 404 | M + 1 |
| P.3 | 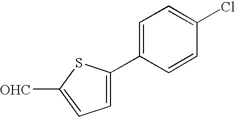 | 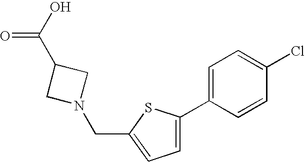 | 1.32 | 308 | M + 1 |
| P.4 | 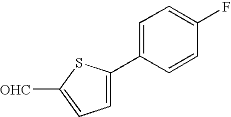 | 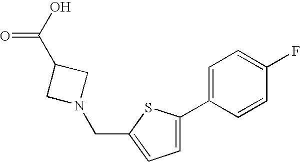 | 1.2 | 292 | M + 1 |
| P.5 | 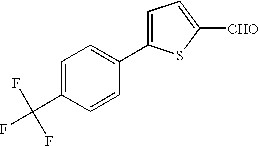 | 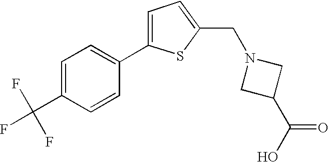 | 1.39 | 342 | M + 1 |
| P.6 | 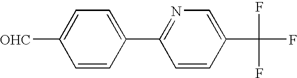 | 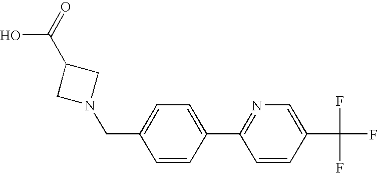 | 1.24 | 337 | M + 1 |
| P.7 | 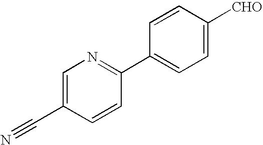 | 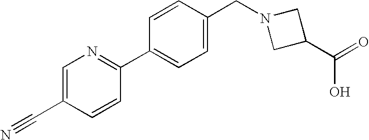 | 0.96 | 294 | M + 1 |
| P.8 | 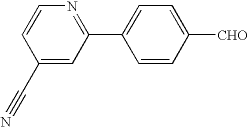 | 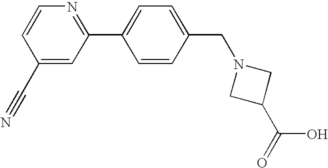 | 0.96 | 294 | M + 1 |
| P.9 | 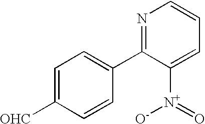 | 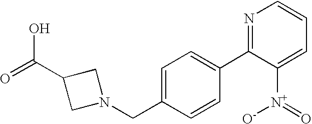 | 0.95 | 314 | M + 1 |
| P.10 | 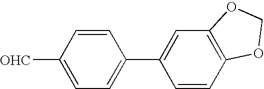 | 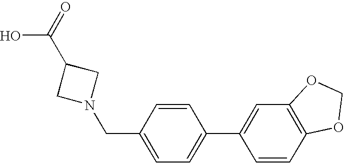 | 1.23 | 312 | M + 1 |
| P.11 | 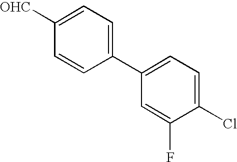 | 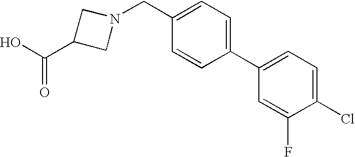 | 1.35 | 320 | M + 1 |
| P.12 | 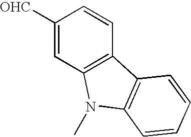 | 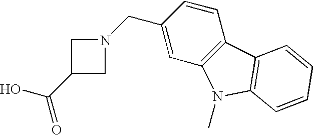 | 1.18 | 295 | M + 1 |
| P.13 | 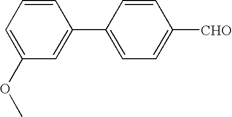 | 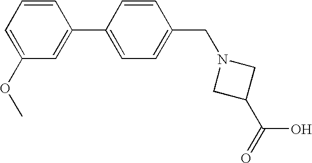 | 1.23 | 298 | M + 1 |
| P.14 | 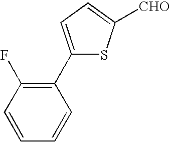 | 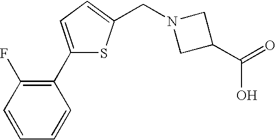 | 1.2 | 292 | M + 1 |
| P.15 | 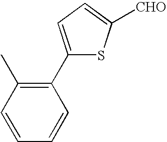 | 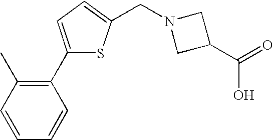 | 1.25 | 288 | M + 1 |
| P.16 | 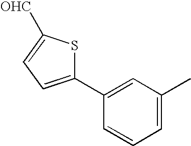 | 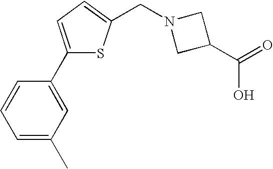 | 1.28 | 288 | M + 1 |
| P.17 | 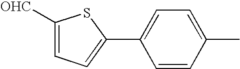 | 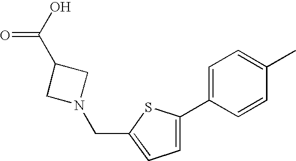 | 1.29 | 288 | M + 1 |
| P.18 | 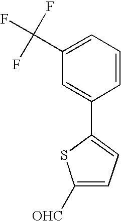 | 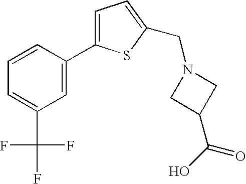 | 1.35 | 342 | M + 1 |
| P.19 | 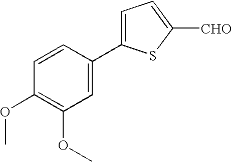 | 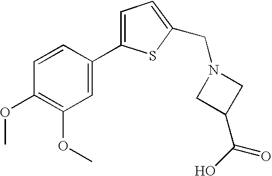 | 1.14 | 334 | M + 1 |
| P.20 | 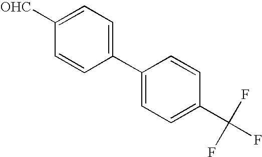 | 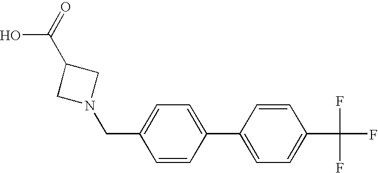 | 1.4 | 336 | M + 1 |
| P.21 | 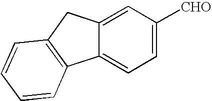 | 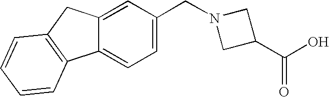 | 1.25 | 280 | M + 1 |
| P.22 | 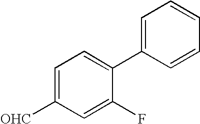 | 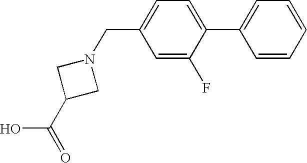 | 1.26 | 285 | M + 1 |
| P.23 | 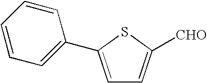 | 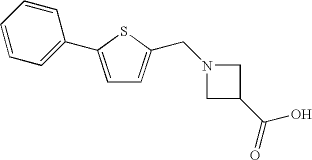 | 1.14 | 274 | M + 1 |
| P.24 | 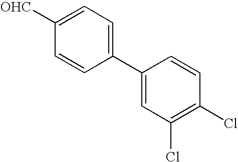 | 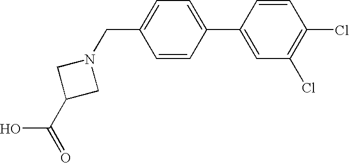 | 1.44 | 335 | M + 1 |
| P.25 | 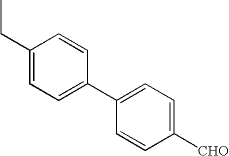 | 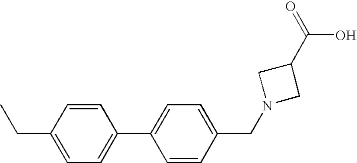 | 1.41 | 296 | M + 1 |
| P.26 | 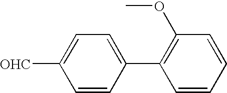 | 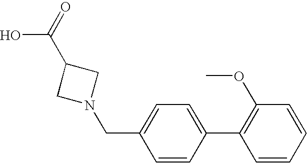 | 1.27 | 298 | M + 1 |
| P.27 | 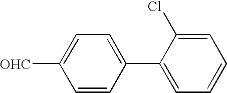 | 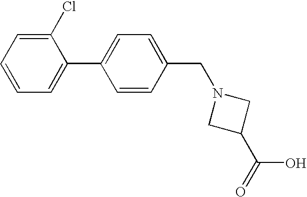 | 1.26 | 302 | M + 1 |
| P.28 | 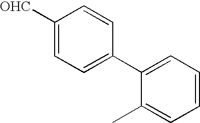 | 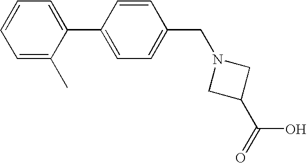 | 1.25 | 282 | M + 1 |

### Example #15

### Synthesis of 1-(4-(Phenylethynyl)benzyl)azetidine-3-carboxylic acid

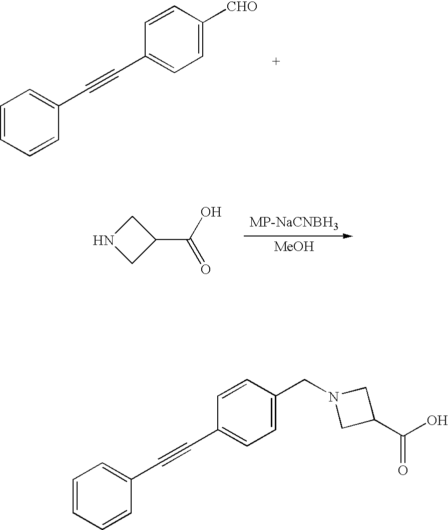

In a 20 mL vial a solution of 4-phenylethynyl-benzaldehyde (49 mg, 0.24 mmol) (Oakwood) dissolved in methanol/dichloromethane (1.5 mL) was added, followed by the addition of azetidine-3-carboxylic acid (20 mg, 1 eq.) dissolved in MeOH/DCM (1.0 mL), and HOAc (3 eq.). The mixture was shaken at about 50° C. for the first about 2 h to which MP-cyanoborohydride resin (5 eq., Biotage) was added. This reaction mixture was allowed to stir overnight at about 50° C. The reaction was checked by LC/MS and concentrated to dryness. The residue was dissolved in 1:1 DMSO/MeOH and purified by reverse phase HPLC. This gave 1-(4-(phenylethynyl)benzyl)azetidine-3-carboxylic acid (15 mg, 0.05 mmol, 26% yield). LC/MS. (Table 2, Method a) Rₜ: 1.34 min; m/z 292 (M+H)⁺.

### Example #16

### Synthesis of (1R,3S)-3-(5-pentylpyrimidin-2-ylamino)cyclopentanecarboxylic acid

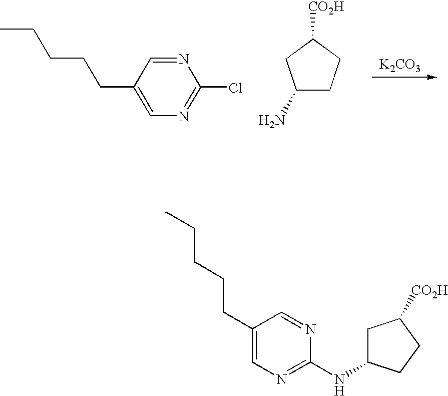

2-Chloro-5-n-pentylpyrimidine (100 mg, 0.542 mmol), (1R,3S)-3-aminocyclopentanecarboxylic acid (84 mg, 0.650 mmol), potassium carbonate (165 mg, 1.191 mmol) and DMSO (2170 μL)/water (1800 were heated in a Biotage microwave at about 170° C. for about 20 min. The reaction mixture was reheated at 170° C. for about 10 min with no significant change in LC/MS. The mixture was cooled down and the reaction mixture was partitioned between DCM (25 mL) and HCl (1M, 25 mL), the aqueous layer was extracted by DCM (25 mL), the combined organic layers were washed with brine (25 mL), filtered through a Biotage Phase separator and concentrated. The crude product was added to a silica gel column and eluted with MeOH/DCM (0-10%, 30 min) Collected fractions containing the correct MW by LC/MS were combined, concentrated and dried in a vacuum oven at about 30° C. to provide (1R,3S)-3-(5-pentylpyrimidin-2-ylamino)cyclopentanecarboxylic acid (54 mg, 0.185 mmol, 34.2% yield) as a viscous oil. ¹H NMR (400 MHz, DMSO-d₆): δ 12.06 (bs, 1H), 8.11 (s, 2H), 6.90 (d, J=7.2 Hz, 1H), 4.23-4.05 (m, 1H), 2.81-2.64 (m, 1H), 2.35 (t, J=7.6 Hz, 2H), 2.19 (dt, J=12.5, 7.3 Hz, 1H), 1.98-1.74 (m, 3H), 1.64 (dt, J=12.6, 9.1 Hz, 1H), 1.58-1.43 (m, 3H), 1.36-1.18 (m, 4H), 0.86 (t, J=7.0 Hz, 3H). LC/MS (Table 2, Method f) Rₜ=1.26 min; MS m/z 278.3 (M+H)⁺.

### Assays

### Inhibition of [33P]S1P Binding to S1P Receptors

Radio-ligand binding was carried out using membranes from transiently transfected HEK cells overexpressing S1P₁, S1P₂, S1P₃, S1P₄ or S1P₅. All compounds were dissolved in DMSO and serial dilutions were carried out in DMSO prior to addition to assay buffer. Final assay DMSO concentrations were 1 or 0.5% (v/v). [33P]S1P was purchased from Perkin Elmer and used at 50 pM in all assays. Frozen membranes were thawed and resuspended in assay buffer containing 50 mM HEPES pH 7.4, 100 mM NaCl, 10 mM MgCl₂ and 0.1% fatty acid free BSA. Membrane was added to give 5-10 μg of membrane per well. Non-specific binding was determined in the presence of cold 1 μM S1P. Incubations were carried out at room temperature for about 45-60 min. After incubation, samples were filtered onto GF/B or GF/C filtration plates using a Packard 96 well harvester. Plates were dried before adding Microscint to each well, sealed and counted on a Topcount (Perkin-Elmer). Radioactivity counts are converted to percent activity, with samples containing [33P]S1P as zero inhibition and samples containing [33P]S1P plus 1 μM S1P as 100% inhibition. IC₅₀'s were determined by fitting the percent activity data to the equation percent activity=100%−(100%/(1+([inhibitor]/IC₅₀))) using non-linear least-means-squares curve fitting.

### S1P Receptor GTPγS Assays

The [³⁵S]GTPγS binding assay was performed using both scintillation proximity assay (SPA) and filtration methods. Both formats are advantageously run in 96 well plates and utilize membranes from stable CHO human cell lines overexpressing S1P₁, S1P₃, S1P₄ or S1P₅. Compound stocks were made up to 10 mM using DMSO and serial dilutions were carried out using 100% DMSO. Compounds were transferred to 96 well plates to yield a final DMSO concentration of 1 or 0.5% (v/v) for all assays. Frozen membranes were thawed and diluted in assay buffer containing of 20 mM HEPES about pH 7.4, 0.1% fatty acid-free BSA, 100 mM NaCl, 5 mM MgCl₂ and 10 μM GDP. For the SPA assay, membranes are premixed with WGA-SPA beads to yield a final concentration per well of 5 μg membrane and 500 μg of bead. For the filtration assay, membranes are added directly to the incubation plate at 5 μg per well. The assay begins with the addition of 50 μl of the membrane or membrane/bead mixture to each well of the assay plate. Next, 50 μl of 0.4 nM [³⁵S]GTPγS is added to each well and incubated for about 30 min. For the SPA assay the plates are spun and then read on the Topcount. For the filtration assay the plate is harvested onto GF-C filtration plates using a Packard 96 well harvester.

S1P Receptor cAMP Assays

Inhibition of forskolin-stimulated cAMP formation was carried out using stable or transient CHO human cell lines overexpressing S1P₁, S1P₂, S1P₃, S1P₄ or S1P₅. All compounds were dissolved in DMSO and serial dilutions were carried out in DMSO prior to addition to assay buffer. Final assay DMSO concentrations are 1% (v/v). After plating, cells were cultured overnight at about 37° C., with 5% CO₂ in Ham F12, 10% heat-inactivated fetal bovine serum, 1% L-glutamine, 1% penicillin-streptomcycin, 1% sodium bicarbonate, and 1 mg/mL G418 sulfate. Alternatively, cells were incubated overnight in FBS-containing media, on the second day media was aspirated, Opti-MEM® I Reduced-Serum Medium (1×) was added, and cells were cultured for an additional two days prior to testing. After removing media, cells were treated with test reagent in 1% DMSO, phosphate-buffered saline without calcium and magnesium, 25 mM HEPES, 0.1% BSA, 0.1 mM IBMX, and 3 μM forskolin. Samples were incubated for about 30 min at room temperature, and all subsequent steps were at room temperature. Buffer was removed and replaced with 60 μL lysis buffer from the HTRF cAMP assay kit, Cis-Us, Inc. After about 60 min incubation with lysis buffer, 40 μL of each well was transferred to a black half-well plate, and 20 μL of detection reagents from the same kit were added and incubated about 2 h before reading on a BMG Labtech RubyStar instrument. Alternatively, after incubation with compounds, lysis buffer and detection reagents were added to the reaction wells without any washing or transfer steps, and plates were read after about 2 h incubation. In a third variation, cells were grown overnight in a flask with OPTI-MEM media. On the second day, cells were harvested with EDTA, washed with PBS/HEPES/BSA, then resuspended in the same buffer and counted. 40,000 cells per well (in 25 μL) were used for the experiment. Compounds, forskolin, and IBMX were added in 25 μL of PBS/HEPES/BSA (final DMSO in the 50 μL was 1%) then incubated for about 30 min at room temperature was followed by addition of 25 μL lysis buffer and 25 μL detection reagents, and then plates were read after about 2 hr as above.

TABLE Q
Assay Data

| | S1P₅ MFB | S1P₁ MFB |
|---|---|---|
| | Potency | Potency |
| Name | Score | Score |
| 1-(4-(3-(Trifluoromethyl)phenethyl) | ++++ | ++ |
| benzyl)azetidine-3-carboxylic acid | | |
| 1-(3-nitro-4-(4- | ++++ | + |
| (trifluoromethyl)phenoxy)benzyl)azetidine- | | |
| 3-carboxylic acid | | |
| 1-(4-(2,4,6-trimethylbenzyloxy)benzyl) | ++++ | ++ |
| azetidine-3-carboxylic acid | | |
| 1-(4-(3,3-Dimethylbut-1- | ++++ | + |
| ynyl)benzyl)azetidine-3-carboxylic acid | | |
| 1-(3-(3,4-dimethylphenoxy)benzyl) | ++++ | + |
| azetidine-3-carboxylic acid | | |
| 1-((5-(phenylethynyl)thiophen-2- | ++++ | + |
| yl)methyl)azetidine-3-carboxylic acid | | |
| 1-((4′-ethylbiphenyl-4-yl)methyl)azetidine-3- | ++++ | ++ |
| carboxylic acid | | |
| 1-(4-(2,4-dichlorobenzyloxy)benzyl) | ++++ | ++ |
| azetidine-3-carboxylic acid | | |
| 1-(4-(3-(trifluoromethyl)benzyloxy)benzyl) | ++++ | + |
| azetidine-3-carboxylic acid | | |
| 1-(4-(3,4-dichlorobenzyloxy)-3- | ++++ | ++ |
| nitrobenzyl)azetidine-3-carboxylic acid | | |
| 1-(4-heptanoylbenzyl)pyrrolidine-3- | ++++ | + |
| carboxylic acid | | |
| 1-(4-(hex-1-ynyl)benzyl)azetidine-3- | ++++ | + |
| carboxylic acid | | |
| 1-(4-((2-cyanothiophen-3-yl)methoxy) | ++++ | + |
| benzyl)azetidine-3-carboxylic acid | | |
| 1-(4-(2-(3,4-dichlorophenyl)acetyl)phenyl) | ++++ | + |
| pyrrolidine-3-carboxylic acid | | |
| 1-(4-(hexyloxycarbonyl)benzyl)azetidine-3- | ++++ | + |
| carboxylic acid | | |
| 1-(4-(2-(3,4-dichlorophenyl)acetyl)benzyl) | ++++ | + |
| azetidine-3-carboxylic acid | | |
| 1-(4-(3-bromobenzyloxy)benzyl)azetidine-3- | ++++ | + |
| carboxylic acid | | |
| 1-(4-(hexyloxy)benzyl)azetidine-3- | ++++ | + |
| carboxylic acid | | |
| 1-(4-(2-chloro-6-fluorobenzyloxy)benzyl) | ++++ | + |
| azetidine-3-carboxylic acid | | |
| 1-(4-(3-chlorobenzyloxy)benzyl)azetidine-3- | +++ | + |
| carboxylic acid | | |
| 1-(4-(4-fluorobenzyloxy)-3- | +++ | + |
| nitrobenzyl)azetidine-3-carboxylic acid | | |
| 1-(4-(4-chlorobenzyloxy)benzyl)azetidine-3- | +++ | + |
| carboxylic acid | | |
| 1-(4-(4-bromobenzyloxy)benzyl)azetidine-3- | +++ | + |
| carboxylic acid | | |
| 1-(4-(hexyloxy)benzyl)piperidine-4- | +++ | + |
| carboxylic acid | | |
| 1-((3′,4′-dichlorobiphenyl-4- | +++ | + |
| yl)methyl)azetidine-3-carboxylic acid | | |
| 1-(4-(2,4-dichlorophenoxy)benzyl)azetidine- | +++ | + |
| 3-carboxylic acid | | |
| 1-(4-(4-fluorobenzoyloxy)-3- | +++ | + |
| methoxybenzyl)azetidine-3-carboxylic acid | | |
| (R)-1-(4-(hexyloxy)benzyl)pyrrolidine-3- | +++ | + |
| carboxylic acid | | |
| 1-(4-(3,4-Dichlorobenzyloxy)benzyl)-3- | +++ | + |
| methylpiperidine-4-carboxylic acid | | |
| 1-(4-(hexyloxy)benzyl)-3-methylpiperidine- | +++ | + |
| 4-carboxylic acid | | |
| 1-(4-(Hexyloxy)benzyl)-4- | +++ | + |
| methylpyrrolidine-3-carboxylic acid | | |
| 1-(3-nitro-4-(3-(trifluoromethyl)phenoxy) | +++ | + |
| benzyl)azetidine-3-carboxylic acid | | |
| 1-(4-(2-Phenylacetyl)benzyl)azetidine-3- | +++ | + |
| carboxylic Acid | | |
| 1-(4-butoxy-3-nitrobenzyl)azetidine-3- | +++ | + |
| carboxylic acid | | |
| 1-(4-(4-bromophenoxy)benzyl)azetidine-3- | +++ | + |
| carboxylic acid | | |
| 1-(4-(2-chloro-4-fluorobenzyloxy)benzyl) | +++ | + |
| azetidine-3-carboxylic acid | | |
| 1-(4-(benzylthio)-3-nitrobenzyl)azetidine-3- | +++ | + |
| carboxylic acid | | |
| (2R,3S)-3-(4-(hexyloxy)benzylamino) | +++ | + |
| bicyclo[2.2.1]hept-5-ene-2-carboxylic acid | | |
| 1-(4-(4-chloro-2-nitrophenoxy)benzyl) | +++ | + |
| azetidine-3-carboxylic acid | | |
| 2-(4-(hexyloxy)benzylamino) | +++ | + |
| cyclopentanecarboxylic acid | | |
| (3R,4S)-1-(4-(hexyloxy)benzyl)pyrrolidine- | +++ | + |
| 3,4-dicarboxylic acid | | |
| 1-(3-methoxy-4-(pentyloxy)benzyl) | +++ | + |
| azetidine-3-carboxylic acid | | |
| 1-(4-(4-chlorophenoxy)benzyl)azetidine-3- | +++ | + |
| carboxylic acid | | |

Legend:
++++ = <0.01 mM
+++ = 0.01-0.099 mM
++ = 0.1-0.99 mM
+ = >1 mM
N.D. = Not Determined

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein.

We claim:

1. A compound represented by Formula (I) 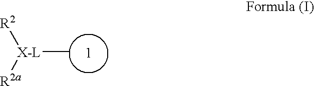
or a pharmaceutically acceptable salt, biologically active metabolite, solvate, hydrate, prodrug, enantiomer or stereoisomer thereof; wherein,
Ring 1 is optionally substituted aryl or optionally substituted heteroaryl;
L is —N(R^{a})—, —O— or —C(R^{a})₂—; wherein
R^{a} is independently H or optionally substituted alkyl;
X is N when L is C(R^{a})₂ or
X is CR^{a}; when L is —N— or —O—;
R² and R^{2a} are independently hydrogen, optionally substituted alkyl, optionally substituted alkenyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹; wherein
W is O or S; and
R¹¹ is —OR, —N(R)₂ or —SR; wherein
R is independently hydrogen, optionally substituted alkyl or haloalkyl; or
when X is N or C, R² and R^{2a} together with the carbon or nitrogen atom to which they are attached form an optionally substituted cycloalkyl, optionally substituted azetidine, optionally substituted pyrrolidine, optionally substituted piperidine or optionally substituted octahydrocyclopenta[c]pyrrolyl ring, provided that the azetidine ring formed by R² and R^{2a} together with the carbon or nitrogen atom to which they are attached is not substituted by
one or more phenyl;
phenyl and OH;
phenyl and —N(H)C(CH₃)₃;
—CH₂—O-optionally substituted pyridinyl;
—NH-optionally substituted quinazolinyl;
—O-optionally substituted pyridinyl;
—O—Si(CH₃)₂—C(CH₃)₃;
—C(OH)(4-(trifluoromethoxy)phenyl)(4-methoxyphenyl);
—C(OH)(4-(trifluoromethoxy)phenyl)(4-methoxyphenyl) and oxo;
—NH-isoquinolinyl;
optionally substituted alkyl and optionally substituted dioxolanyl;
oxo and —O-alkenyl;
oxo, two F and optionally substituted phenyl;
optionally substituted alkenyl and —O—C(O)-optionally substituted phenyl;
provided that when Ring 1 is optionally substituted phenyl, L is CH₂ X is N or C, and R² and R^{2a} together with the carbon or nitrogen atom to which they are attached form an optionally substituted cycloalkyl, or optionally substituted azetidine, Ring 1 is not substituted by
—CH═N—OCH₂CH₃;
—Cl and —NH₂;
—C(═O)CH₂CH₂-optionally substituted oxazolyl;
—NH—C(O)-alkenyl-optionally substituted pyridinyl;
—NO₂ and COOH—O-alkyl-optionally substituted oxazolyl;
—O—CH₂-optionally substituted benzofuranyl;
—O—CH₂-optionally substituted phenyl;
—O—CH₂-optionally substituted pyrazolyl;
—O—CH₂-optionally substituted thienyl;
—O-optionally substituted (C₈)alkyl;
—O-optionally substituted (C₈)alkyl and halo;
—(C₆-C₁₂)alkyl wherein one or more carbons is optionally replaced by a nonperoxide oxygen;
—(C₆-C₁₂)alkenyl wherein one or more carbons is optionally replaced by a nonperoxide oxygen;
-pyrimidinyl substituted with oxo and —CF₂CF₃;
-optionally substituted 1,2,4 oxadiazole;
-optionally substituted thiazolo[5,4-b]pyridine;
-optionally substituted phenyl-CH₂—C(O)-optionally substituted pyrazolyl;
-optionally substituted phenyl-CH₂—C(O)-optionally substituted thiazolyl;
-optionally substituted phenyl-NH—C(O)-optionally substituted pyrazolyl;
-optionally substituted phenyl-NH—C(O)-optionally substituted tetrazolyl;
-optionally substituted phenyl-NH—C(O)-optionally substituted triazolyl;
-optionally substituted pyridinyl-CH₂—C(O)-optionally substituted pyrazolyl;
-optionally substituted pyridinyl-CH₂—C(O)-optionally substituted thiazolyl;
-optionally substituted pyridinyl-NH—C(O)-optionally substituted pyrazolyl;
-optionally substituted pyridinyl-NH—C(O)-optionally substituted tetrazoyl;
-optionally substituted pyridinyl-NH—C(O)-optionally substituted triazolyl;
-optionally substituted pyrimidinyl-CH₂—C(O)-optionally substituted pyrazolyl;
-optionally substituted pyrimidinyl-NH—C(O)-optionally substituted pyrazolyl;
-optionally substituted pyrimidinyl-NH—C(O)-optionally substituted triazolyl;
-optionally substituted phenyl-CH₂—C(O)-optionally substituted triazolyl;
provided that when Ring 1 is optionally substituted isoxazolyl or optionally substituted oxazolyl, Ring 1 is not substituted by
-optionally substituted phenyl-optionally substituted bicycle[2.2.1]heptanyl;
-optionally substituted phenyl-optionally substituted alkyl-optionally substituted phenyl;
provided that when Ring 1 is optionally substituted pyridinyl, Ring 1 is not substituted by
—C(O)—NH-optionally substituted phenyl;
—O-optionally substituted phenyl; and
provided that when Ring 1 is optionally substituted phenyl or naphthyl, L is CH₂ and NR² and NR^{2a} form an optionally substituted pyrrolidine ring, the pyrrolidine ring is not substituted by
—C(═O)(OH);
—F and —C(═O)(OH);
—OH and —C(═O)(OH);
—P(═O)(OH)(OH);
—OH and —P(═O)(OH)(OH);
—CH₂C(═O)(OH); or
tetrazolyl.

2. The compound of claim 1 wherein Ring 1 is optionally substituted benzofuranyl, optionally substituted benzimidazolyl, optionally substituted dibenzofuranyl, optionally substituted benzothiazolyl, optionally substituted benzothienyl, 9H-carbazolyl, optionally substituted cinnolinyl, optionally substituted fluorenyl, optionally substituted furanyl, optionally substituted imidazolyl, optionally substituted indazolyl, optionally substituted indenyl, optionally substituted indolizinyl, optionally substituted indolyl, optionally substituted isoindolyl, optionally substituted 3H-indolyl, optionally substituted isothiazolyl, optionally substituted isoxazolyl, optionally substituted naphthyridinyl, optionally substituted naphthalenyl, optionally substituted oxadiazolyl, optionally substituted oxazolyl, optionally substituted phthalazinyl, optionally substituted pteridinyl, optionally substituted purinyl, optionally substituted phenyl, optionally substituted pyrazolyl, optionally substituted pyridazinyl, optionally substituted pyridinyl, optionally substituted pyrimidinyl, optionally substituted pyrrolyl, optionally substituted quinazolinyl, optionally substituted quinoxalinyl, optionally substituted quinolizinyl, optionally substituted quinolinyl, optionally substituted isoquinolinyl, optionally substituted tetrazolyl, optionally substituted thienyl, or optionally substituted triazolyl.

3. The compound of claim 2 wherein -L-X(R²)(R^{2a}) form 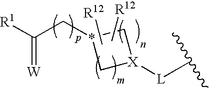
wherein
R¹ is hydrogen, hydroxy, optionally substituted alkyl, optionally substituted alkoxy, haloalkoxy or haloalkyl, —(CH₂)ₓ—O—P(═O)(OR⁷)(OR⁷), —(CH₂)ₓ—P(═O)(OR⁷)(OR⁷), —(CH₂)ₓ—P(═O)(OR⁷)(R⁷), —CH═CH—P—(═O)(OR⁷)(OR⁷);
wherein R⁷ is hydrogen, optionally substituted alkyl or optionally substituted phenyl; and
x is 0 or 1;
R^{a} is hydrogen, optionally substituted alkyl or haloalkyl;
R¹² is independently hydrogen, hydroxy, optionally substituted alkyl, halo, or —(CH₂)ₚC(═W)R¹¹;
m is 1, 2 or 3;
n is 0, 1 or 2 and
p is 0 or 1.

4. The compound of claim 3 wherein the compound is
1-((1-(phenylsulfonyl)-1H-indol-3-yl)methyl)azetidine-3-carboxylic acid;
1-(1-(9H-carbazol-2-yl)ethyl)azetidine-3-carboxylic acid;
1-(dibenzo[b,d]furan-3-ylmethyl)azetidine-3-carboxylic acid;
1-((5-(phenylethynyl)thiophen-2-yl)methyl)azetidine-3-carboxylic acid;
1-((2-(4-methoxybenzoyl)benzofuran-5-yl)methyl)azetidine-3-carboxylic acid;
1-((5-(4-bromophenyl)isoxazol-3-yl)methyl)azetidine-3-carboxylic acid;
1-((6-(3,4-dichlorophenyl)pyridin-3-yl)methyl)azetidine-3-carboxylic acid;
1-((6-(4-(trifluoromethyl)phenyl)pyridin-3-yl)methyl)azetidine-3-carboxylic acid;
1-((6-(benzyloxy)pyridin-3-yl)methyl)azetidine-3-carboxylic acid;
1-((6-(3,4-dichlorobenzyloxy)pyridin-3-yl)methyl)azetidine-3-carboxylic acid;
1-((5-(4-methoxyphenyl)thiophen-2-yl)methyl)azetidine-3-carboxylic acid;
1-((5-(4-chlorophenyl)thiophen-2-yl)methyl)azetidine-3-carboxylic acid;
1-((5-(4-fluorophenyl)thiophen-2-yl)methyl)azetidine-3-carboxylic acid;
1-((5-(4-(trifluoromethyl)phenyl)thiophen-2-yl)methyl)azetidine-3-carboxylic acid;
1-((5-(4-fluorophenyl)thiophen-2-yl)methyl)azetidine-3-carboxylic acid;
1-((5-o-tolylthiophen-2-yl)methyl)azetidine-3-carboxylic acid;
1-((5-m-tolylthiophen-2-yl)methyl)azetidine-3-carboxylic acid;
1-((5-p-tolylthiophen-2-yl)methyl)azetidine-3-carboxylic acid;
1-((5-(3-(trifluoromethyl)phenyl)thiophen-2-yl)methyl)azetidine-3-carboxylic acid;
1-((5-(3,4-dimethoxyphenyl)thiophen-2-yl)methyl)azetidine-3-carboxylic acid;
1-((5-phenylthiophen-2-yl)methyl)azetidine-3-carboxylic acid;
1-((3′,4′-dichlorobiphenyl-4-yl)methyl)azetidine-3-carboxylic acid;
1-((4′-ethylbiphenyl-4-yl)methyl)azetidine-3-carboxylic acid;
1-((2′-methoxybiphenyl-4-yl)methyl)azetidine-3-carboxylic acid;
1-((2′-chlorobiphenyl-4-yl)methyl)azetidine-3-carboxylic acid;
1-((2′-methylbiphenyl-4-yl)methyl)azetidine-3-carboxylic acid;
1-((6-(3-(trifluoromethyl)benzyloxy)pyridin-3-yl)methyl)azetidine-3-carboxylic acid;
2-(2-fluoro-4-(3-(trifluoromethyl)benzyloxy)benzyl)octahydrocyclopenta[c]pyrrole-3a-carboxylic acid; or
ethyl 4-(4-(benzyloxy)phenoxy)cyclohexanecarboxylate.

5. The compound of claim 3 wherein the compound is 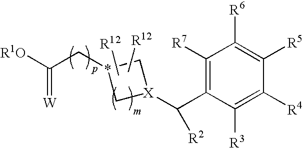
wherein
R³, R⁴, R⁶, and R⁷ are independently selected from the group consisting of optionally substituted alkenyl, optionally substituted alkoxy, optionally substituted alkoxycarbonyl, optionally substituted alkoxysulfonyl, optionally substituted alkyl, optionally substituted alkylcarbonyl, optionally substituted alkylcarbonyloxy, optionally substituted alkylsulfonyl, optionally substituted alkylthio, optionally substituted alkynyl, optionally substituted aryl, optionally substituted aryloxy, amido, optionally substituted amino, carboxy, cyano, formyl, halo, haloalkoxy, haloalkyl, hydrogen, hydroxyl, hydroxyalkyl, mercapto, nitro, silyl and silyloxy;
R⁵ is optionally substituted aryl, optionally substituted arylalkyl, optionally substituted arylalkylcarbonyl, optionally substituted 2-thiazolyl, optionally substituted arylalkoxy, optionally substituted arylalkylthio, optionally substituted arylcarbonyloxy, optionally substituted arylcarbonylalkoxy, optionally substituted aryloxycarbonyl, optionally substituted arylalkenyl, optionally substituted alkyl, optionally substituted alkylcarbonyl, optionally substituted alkenyl, optionally substituted alkynyl, optionally substituted alkenyloxy, optionally substituted aryloxy, optionally substituted alkoxy, optionally substituted alkoxycarbonyl, haloalkoxy, optionally substituted cycloalkoxy, optionally substituted alkenyloxy, optionally substituted arylalkynyl, optionally substituted benzo[d][1,3]dioxolyl, optionally substituted cycloalkyl, optionally substituted cycloalkyloxy, optionally substituted heterarylalkyl, optionally substituted heteroaryl or optionally substituted heteroarylalkyloxy.

6. The compound of claim 5 wherein R⁵ is halogen, optionally substituted alkyl, optionally substituted alkynyl, optionally substituted alkoxy, optionally substituted alkenyloxy, optionally substituted alkyloxycarbonyl, optionally substituted benzo[d][1,3]dioxolyl, optionally substituted benzyl, optionally substituted benzylcarbonyl, optionally substituted benzylthio, optionally substituted benzyloxy, optionally substituted cycloalkyloxy, optionally substituted naphthyl, optionally substituted aryl, optionally substituted arylalkenyl, optionally substituted arylcarbonyloxy, optionally substituted arylalkyl, optionally substituted aryloxy, optionally substituted pyridinyl, optionally substituted thiazolyl, optionally substituted thienyl, or optionally substituted thienylalkoxy.

7. The compound of claim 6 wherein R⁵ is halogen, optionally substituted alkyl, optionally substituted alkynyl, optionally substituted alkoxy, optionally substituted alkenyloxy, optionally substituted alkyloxycarbonyl, optionally substituted benzo[d][1,3]dioxolyl, optionally substituted benzyl, optionally substituted benzylcarbonyl, optionally substituted benzylthio, optionally substituted benzyloxy, optionally substituted cycloalkyloxy, optionally substituted naphthyl, optionally substituted phenyl, optionally substituted phenylalkenyl, optionally substituted phenylcarbonyloxy, optionally substituted phenylethyl, optionally substituted phenyoxy, optionally substituted pyridinyl, optionally substituted thiazolyl, optionally substituted thienyl, or optionally substituted thienylalkoxy.

8. The compound of claim 7 wherein R⁵ is optionally substituted by one or more substituents independently selected from the group consisting of —C(O)-optionally substituted alkyl, —C(O)-optionally substituted alkoxy, —C(O)-optionally substituted phenyl, —O-optionally substituted cycloalkyl, optionally substituted alkoxy, optionally substituted alkyl, halo, CF₃, cyano, nitro, oxo, optionally substituted phenyl, trimethylsilylalkynyl,

9. The compound of claim 8 wherein the compound is
1-((4′-methylbiphenyl-4-yl)methyl)azetidine-3-carboxylic acid;
1-(4-(2-chlorobenzyloxy)-3-methoxybenzyl)azetidine-3-carboxylic acid;
1-(4-(2-chlorobenzyloxy)-3-ethoxybenzyl)azetidine-3-carboxylic acid;
1-(4-(4-chlorobenzyloxy)-3-ethoxybenzyl)azetidine-3-carboxylic acid;
1-(4-(2-methylbenzyloxy)benzyl)azetidine-3-carboxylic acid;
1-(4-(4-fluorobenzyloxy)-3-methoxybenzyl)azetidine-3-carboxylic acid;
1-(4-(benzyloxy)-3-ethoxybenzyl)azetidine-3-carboxylic acid;
1-(1-(biphenyl-4-yl)ethyl)azetidine-3-carboxylic acid;
1-(1-(4′-methylbiphenyl-4-yl)ethyl)azetidine-3-carboxylic acid;
1-(1-(4′-chlorobiphenyl-4-yl)ethyl)azetidine-3-carboxylic acid;
1-(1-(3′-methoxybiphenyl-4-yl)ethyl)azetidine-3-carboxylic acid;
1-(1-(3′-(trifluoromethyl)biphenyl-4-yl)ethyl)azetidine-3-carboxylic acid;
1-(4-(benzylthio)-3-nitrobenzyl)azetidine-3-carboxylic acid;
1-(4-(4-fluorobenzyloxy)benzyl)azetidine-3-carboxylic acid;
1-(4-(hex-1-ynyl)benzyl)azetidine-3-carboxylic acid;
1-(4-pentylbenzyl)azetidine-3-carboxylic acid;
1-(1-(4-(2-chloro-6-fluorobenzyloxy)benzyl)azetidine-3-carboxylic acid;
1-(1-(4-(benzyloxy)-3-chlorobenzyl)azetidine-3-carboxylic acid;
1-(4-(2-chlorobenzyloxy)benzyl)azetidine-3-carboxylic acid;
1-(1-(4-(4-(methoxycarbonyl)benzyloxy)benzyl)azetidine-3-carboxylic acid;
1-(1-(4-(3-fluorobenzyloxy)benzyl)azetidine-3-carboxylic acid;
1-(1-(4-(2,4-dichlorobenzyloxy)benzyl)azetidine-3-carboxylic acid;
1-(1-(4-(2-methylbenzyloxy)benzyl)azetidine-3-carboxylic acid;
1-(4-hexylbenzyl)azetidine-3-carboxylic acid;
1-(4-((trimethylsilyl)ethynyl)benzyl)azetidine-3-carboxylic acid;
1-(4-(benzyloxy)-2-methylbenzyl)azetidine-3-carboxylic acid;
1-(4-(benzyloxy)-3,5-dimethylbenzyl)azetidine-3-carboxylic acid;
1-(4-(4-bromobenzyloxy)benzyl)azetidine-3-carboxylic acid;
1-(4-(2-chloro-6-fluorobenzyloxy)benzyl)azetidine-3-carboxylic acid;
1-(4-(benzyloxy)-3-chlorobenzyl)azetidine-3-carboxylic acid;
1-(4-(3-(methoxycarbonyl)benzyloxy)benzyl)azetidine-3-carboxylic acid;
1-(4-(4-chlorobenzyloxy)-3-methoxybenzyl)azetidine-3-carboxylic acid;
1-(4-(2-chlorobenzyloxy)-3-methoxybenzyl)azetidine-3-carboxylic acid;
1-(3-methoxy-4-(4-methylbenzyloxy)benzyl)azetidine-3-carboxylic acid;
1-(4-(2-chlorobenzyloxy)-3-ethoxybenzyl)azetidine-3-carboxylic acid;
1-(4-(4-chlorobenzyloxy)benzyl)azetidine-3-carboxylic acid;
1-(4-(2-chloro-benzyloxy)benzyl)azetidine-3-carboxylic acid;
1-(4-(2-chloro-4-fluorobenzyloxy)benzyl)azetidine-3-carboxylic acid;
1-(4-(3-methylbenzyloxy)benzyl)azetidine-3-carboxylic acid;
1-(4-(3-(trifluoromethyl)benzyloxy)benzyl)azetidine-3-carboxylic acid;
1-(4-(3-methoxybenzyloxy)benzyl)azetidine-3-carboxylic acid;
1-(4-(3-bromobenzyloxy)benzyl)azetidine-3-carboxylic acid;
1-(4-(4-chlorobenzyloxy)-3-ethoxybenzyl)azetidine-3-carboxylic acid;
1-(4-(4-nitrobenzoyloxy)benzyl)azetidine-3-carboxylic acid;
1-(4-(4-fluorobenzoyloxy)-3-methoxybenzyl)azetidine-3-carboxylic acid;
1-(4-(2,4-dichlorobenzyloxy)-3-methoxybenzyl)azetidine-3-carboxylic acid;
1-(4-(benzyloxy)-3,5-dibromobenzyl)azetidine-3-carboxylic acid;
1-(4-(benzyloxy)-3-bromo-5-methoxybenzyl)azetidine-3-carboxylic acid;
1-(4-(benzyloxy)-3,5-dimethoxybenzyl)azetidine-3-carboxylic acid;
1-(4-(2-fluorobenzyloxy)benzyl)azetidine-3-carboxylic acid;
1-(4-(3-fluorobenzyloxy)benzyl)azetidine-3-carboxylic acid;
1-(4-(2,4-dichlorobenzyloxy)benzyl)azetidine-3-carboxylic acid;
1-(4-(2-methylbenzyloxy)benzyl)azetidine-3-carboxylic acid;
1-(4-(4-fluorobenzyloxy)-3-methoxybenzyl)azetidine-3-carboxylic acid;
1-(4-(2,4,6-trimethylbenzyloxy)benzyl)azetidine-3-carboxylic acid;
1-(4-(2-methoxy-2-oxo-1-phenylethoxy)benzyl)azetidine-3-carboxylic acid;
1-(4-(2-(methoxycarbonyl)-6-nitrobenzyloxy)benzyl)azetidine-3-carboxylic acid;
1-(4-(4-fluorobenzyloxy)-3-nitrobenzyl)azetidine-3-carboxylic acid;
1-(4-(3,4-dichlorobenzyloxy)-3-nitrobenzyl)azetidine-3-carboxylic acid;
1-(4-(benzyloxy)-3-ethoxybenzyl)azetidine-3-carboxylic acid;
1-(4-(3,4,5-trimethoxybenzoyloxy)benzyl)azetidine-3-carboxylic acid;
1-(4-(4-methylbenzyloxy)benzyl)azetidine-3-carboxylic acid;
1-(4-(3-chlorobenzyloxy)benzyl)azetidine-3-carboxylic acid;
1-(4-butoxybenzyl)azetidine-3-carboxylic acid;
1-(4-(pentyloxy)benzyl)azetidine-3-carboxylic acid;
1-(4-(isopentyloxy)benzyl)azetidine-3-carboxylic acid;
1-(4-pentylbenzyl)azetidine-3-carboxylic acid;
1-(4-(4-chlorophenoxy)benzyl)azetidine-3-carboxylic acid;
1-(4-butoxy-3-nitrobenzyl)azetidine-3-carboxylic acid;
1-(4-(2,4-dichlorophenoxy)benzyl)azetidine-3-carboxylic acid;
1-(4-(4-methoxyphenoxy)benzyl)azetidine-3-carboxylic acid;
1-(4-(4-bromophenoxy)benzyl)azetidine-3-carboxylic acid;
1-(4-(3-chlorophenoxy)benzyl)azetidine-3-carboxylic acid;
1-(4-(3,4-dimethylphenoxy)benzyl)azetidine-3-carboxylic acid;
1-(4-(4-tert-butylphenoxy)-3-nitrobenzyl)azetidine-3-carboxylic acid;
1-(4-(4-chloro-2-nitrophenoxy)benzyl)azetidine-3-carboxylic acid;
1-(4-(4-fluorophenoxy)-3-nitrobenzyl)azetidine-3-carboxylic acid;
1-(3-nitro-4-(3-(trifluoromethyl)phenoxy)benzyl)azetidine-3-carboxylic acid;
1-(3-nitro-4-(p-tolyloxy)benzyl)azetidine-3-carboxylic acid;
1-(4-(2,4-difluorophenoxy)-3-nitrobenzyl)azetidine-3-carboxylic acid;
1-(4-cyclopentyloxy)-3-methoxybenzyl)azetidine-3-carboxylic acid;
1-(4-(cyclopentyloxy)benzyl)azetidine-3-carboxylic acid;
1-(4-butoxy-3-methoxybenzyl)azetidine-3-carboxylic acid;
1-(4-(hexyloxy)benzyl)piperidine-4-carboxylic acid;
(S)-2-(1-(4-(hexyloxy)benzyl)pyrrolidin-2-yl)acetic acid;
(R)-1-(4-(hexyloxy)benzyl)pyrrolidine-3-carboxylic acid;
(R)-1-(4-(hexyloxy)benzyl)piperidine-3-carboxylic acid;
(S)-1-(4-(hexyloxy)benzyl)piperidine-3-carboxylic acid;
1-(4-(hexyloxy)benzyl)-3-methylpiperidine-4-carboxylic acid;
1-(4-(hexyloxy)benzyl)pyrrolidine-3-carboxylic acid;
(3R,4S)-1-(4-(hexyloxy)benzyl)pyrrolidine-3,4-dicarboxylic acid;
1-(4-phenoxybenzyl)azetidine-3-carboxylic acid;
1-(4-(benzyloxy)benzyl)azetidine-3-carboxylic acid;
1-(4-propoxybenzyl)azetidine-3-carboxylic acid;
1-(4-butoxybenzyl)azetidine-3-carboxylic acid;
1-(4-(4-tent-butylthiazol-2-yl)benzyl)azetidine-3-carboxylic acid;
1-(4-(benzyloxy)-3-methoxybenzyl)azetidine-3-carboxylic acid;
(E)-1-(4-styrylbenzyl)azetidine-3-carboxylic acid;
1-(4-(hexyloxy)benzyl)azetidine-3-carboxylic acid;
1-(4-butylbenzyl)azetidine-3-carboxylic acid;
1-(4-(allyloxy)benzyl)azetidine-3-carboxylic acid;
1-((2-fluorobiphenyl-4-yl)methyl)azetidine-3-carboxylic acid;
1-(4-(thiophen-2-yl)benzyl)azetidine-3-carboxylic acid;
1-((biphenyl-4-yl)methyl)azetidine-3-carboxylic acid;
1-(3,4-bis(benzyloxy)benzyl)azetidine-3-carboxylic acid;
1-(4-(benzyloxy)-2-methoxybenzyl)azetidine-3-carboxylic acid;
1-(4-isobutylbenzyl)azetidine-3-carboxylic acid;
1-((3′,4′-dichlorobiphenyl-4-yl)methyl)azetidine-3-carboxylic acid;
1-(4-(pentyloxy)benzyl)azetidine-3-carboxylic acid;
1-(3-ethoxy-4-(heptyloxy)benzyl)azetidine-3-carboxylic acid;
1-(4-(isopentyloxy)benzyl)azetidine-3-carboxylic acid;
1-(4-(2-(3,4-dimethylphenyl)-2-oxoethoxy)benzyl)azetidine-3-carboxylic acid;
1-(3-methoxy-4-(pentyloxy)benzyl)azetidine-3-carboxylic acid;
1-(4-butoxy-3-ethoxybenzyl)azetidine-3-carboxylic acid;
1-(3-bromo-5-methoxy-4-propoxybenzyl)azetidine-3-carboxylic acid;
1-(3-chloro-5-methoxy-4-propoxybenzyl)azetidine-3-carboxylic acid;
1-(4-isobutoxy-3-ethoxybenzyl)azetidine-3-carboxylic acid;
1-(4-(isopentyloxy)-3-methoxybenzyl)azetidine-3-carboxylic acid;
1-(4-(3-fluoropropoxy)benzyl)azetidine-3-carboxylic acid;
1-(4-((2-cyanothiophen-3-yl)methoxy)benzyl)azetidine-3-carboxylic acid;
1-((4′-ethylbiphenyl-4-yl)methyl)azetidine-3-carboxylic acid;
1-((2′-methoxybiphenyl-4-yl)methyl)azetidine-3-carboxylic acid;
1-((3′,5′-dichlorobiphenyl-4-yl)methyl)azetidine-3-carboxylic acid;
1-((3′-chlorobiphenyl-4-yl)methyl)azetidine-3-carboxylic acid;
1-((3′,4′-dimethylbiphenyl-4-yl)methyl)azetidine-3-carboxylic acid;
1-((3′-methylbiphenyl-4-yl)methyl)azetidine-3-carboxylic acid;
1-(4-(3,4-dichlorobenzyloxy)benzyl)azetidine-3-carboxylic acid;
1-(4-(4-chlorophenoxy)benzyl)azetidine-3-carboxylic acid;
1-((3′-(trifluoromethyl)biphenyl-4-yl)methyl)azetidine-3-carboxylic acid;
1-(4-(naphthalen-1-yl)benzyl)azetidine-3-carboxylic acid;
1-(4-(3,4-dichlorobenzyloxy)benzyl)-3-methylpiperidine-4-carboxylic acid;
1-(4-(3,4-dichlorobenzyloxy)benzyl)azetidine-3-carboxylic acid;
1-(4-(hexyloxycarbonyl)benzyl)azetidine-3-carboxylic acid;
1-(4-(hexyloxy)benzyl)-4-methylpyrrolidine-3-carboxylic acid;
1-(4-(3,4-dichlorobenzyloxy)-3-nitrobenzyl)azetidine-3-carboxylic acid;
1-(4-(hexyloxy)-3-methoxybenzyl)azetidine-3-carboxylic acid;
1-(4-(2-phenylacetyl)benzyl)azetidine-3-carboxylic acid;
1-(4-phenethylbenzyl)azetidine-3-carboxylic acid;
1-(4-(2-(3-(trifluoromethyl)phenyl)acetyl)benzyl)azetidine-3-carboxylic acid;
1-(4-(benzyloxy)-3-fluorobenzyl)azetidine-3-carboxylic acid;
1-(4-(benzyloxy)-2-chlorobenzyl)azetidine-3-carboxylic acid;
1-(4-(benzyloxy)-2-fluorobenzyl)azetidine-3-carboxylic acid;
1-(4-(benzyloxy)-3-chlorobenzyl)azetidine-3-carboxylic acid;
1-(3-fluoro-4-(3-(trifluoromethyl)benzyloxy)benzyl)azetidine-3-carboxylic acid;
1-(2-chloro-4-(3-(trifluoromethyl)benzyloxy)benzyl)azetidine-3-carboxylic acid;
1-(2-fluoro-4-(3-(trifluoromethyl)benzyloxy)benzyl)azetidine-3-carboxylic acid;
1-(3-chloro-4-(3-(trifluoromethyl)benzyloxy)benzyl)azetidine-3-carboxylic acid;
1-(4-(3,4-dichlorobenzyloxy)-3-fluorobenzyl)azetidine-3-carboxylic acid;
1-(4-(3,4-dichlorobenzyloxy)-2-fluorobenzyl)azetidine-3-carboxylic acid;
1-(4-(3,4-dichlorobenzyloxy)-2-methylbenzyl)azetidine-3-carboxylic acid;
1-(4-(benzyloxy)-2-methylbenzyl)azetidine-3-carboxylic acid;
1-(2-methyl-4-(3-(trifluoromethyl)benzyloxy)benzyl)azetidine-3-carboxylic acid;
1-(4-(1-phenylethoxy)benzyl)azetidine-3-carboxylic acid;
(R)-1-(4-(1-phenylethoxy)benzyl)azetidine-3-carboxylic acid;
(S)-1-(4-(1-phenylethoxy)benzyl)azetidine-3-carboxylic acid;
1-(4-(2-phenylacetyl)benzyl)pyrrolidine-3-carboxylic acid;
1-(4-(2-phenylacetyl)benzyl)pyrrolidine-3-carboxylic acid;
1-(4-(2-(3,4-dichlorophenyl)acetyl)benzyl)azetidine-3-carboxylic acid;
1-(4-(2-(3,4-dichlorophenyl)acetyl)phenyl)pyrrolidine-3-carboxylic acid;
1-(4-hexanoylbenzyl)azetidine-3-carboxylic acid;
1-(4-hexanoylbenzyl)pyrrolidine-3-carboxylic acid;
(1R,3S)-3-((6-hexanoylpyridin-3-yl)methylamino)cyclopentanecarboxylic acid;
1-(4-heptanoylbenzyl)azetidine-3-carboxylic acid;
1-(4-heptanoylbenzyl)pyrrolidine-3-carboxylic acid;
3-(4-heptanoylbenzyl)cyclopentanecarboxylic acid;
1-(4-(3,3-dimethylbut-1-ynyl)benzyl)azetidine-3-carboxylic acid;
1-(2-fluoro-4-(3-(trifluoromethyl)benzyloxy)benzyl)piperidine-4-carboxylic acid;
1-(2-fluoro-4-(3-(trifluoromethyl)benzyloxy)benzyl)piperidine-3-carboxylic acid;
3-(4-(benzyloxy)phenylamino)cyclopentanecarboxylic acid;
1-(1-(4-(benzyloxy)phenyl)ethyl)azetidine-3-carboxylic acid;
1-(4-((trimethylsilyl)ethynyl)benzyl)piperidine-4-carboxylic acid;
1-(4-((trimethylsilyl)ethynyl)benzyl)pyrrolidine-3-carboxylic acid;
1-(4-((trimethylsilyl)ethynyl)benzyl)piperidine-3-carboxylic acid;
4,4-dimethyl-1-(4-((trimethylsilyl)ethynyl)benzyl)pyrrolidine-3-carboxylic acid;
4-methyl-1-(4-((trimethylsilyl)ethynyl)benzyl)pyrrolidine-3-carboxylic acid;
1-((3′,5′-bis(trifluoromethyl)biphenyl-4-yl)methyl)azetidine-3-carboxylic acid;
1-(4-(5-(trifluoromethyl)pyridin-2-yl)benzyl)azetidine-3-carboxylic acid;
1-(4-(5-cyanopyridin-2-yl)benzyl)azetidine-3-carboxylic acid;
1-(4-(4-cyanopyridin-2-yl)benzyl)azetidine-3-carboxylic acid;
1-(4-(3-nitropyridin-2-yl)benzyl)azetidine-3-carboxylic acid;
1-(4-(benzo[d][1,3]dioxol-5-yl)benzyl)azetidine-3-carboxylic acid;
1-(4-chloro-3-fluorobenzyl)azetidine-3-carboxylic acid;
1-((9-methyl-9H-carbazol-2-yl)methyl)azetidine-3-carboxylic acid;
1-((3′-methoxybiphenyl-4-yl)methyl)azetidine-3-carboxylic acid;
1-(4′-(trifluoromethyl)biphenyl-4-yl)methyl)azetidine-3-carboxylic acid;
1-((9H-fluoren-3-yl)methyl)azetidine-3-carboxylic acid;
1-((2-fluorobiphenyl-4-yl)methyl)azetidine-3-carboxylic acid; or
1-(4-(phenylethynyl)benzyl)azetidine-3-carboxylic acid.

10. The compound of claim 2 wherein the compound is 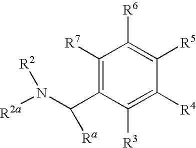
wherein
R³, R⁴, R⁶, and R⁷ are independently selected from the group consisting of optionally substituted alkenyl, optionally substituted alkoxy, optionally substituted alkoxycarbonyl, optionally substituted alkoxysulfonyl, optionally substituted alkyl, optionally substituted alkylcarbonyl, optionally substituted alkylcarbonyloxy, optionally substituted alkylsulfonyl, optionally substituted alkylthio, optionally substituted alkynyl, optionally substituted aryl, optionally substituted aryloxy, amido, optionally substituted amino, carboxy, cyano, formyl, halo, haloalkoxy, haloalkyl, hydrogen, hydroxyl, hydroxyalkyl, mercapto, nitro, silyl and silyloxy;
R⁵ is optionally substituted aryl, optionally substituted arylalkyl, optionally substituted arylalkylcarbonyl, optionally substituted 2-thiazolyl, optionally substituted arylalkoxy, optionally substituted arylalkylthio, optionally substituted arylcarbonyloxy, optionally substituted arylcarbonylalkoxy, optionally substituted aryloxycarbonyl, optionally substituted arylalkenyl, optionally substituted arylalkyl, optionally substituted alkyl, optionally substituted alkylcarbonyl, optionally substituted alkenyl, optionally substituted alkynyl, optionally substituted alkenyloxy, optionally substituted aryloxy, optionally substituted aryloxycarbonyl, optionally substituted alkoxy, optionally substituted alkoxycarbonyl, haloalkoxy, optionally substituted cycloalkoxy, optionally substituted alkenyloxy, optionally substituted arylalkynyl, optionally substituted cycloalkyl, optionally substituted cycloalkyloxy, optionally substituted heterarylalkyl or optionally substituted heteroaryl.

11. The compound of claim 10 wherein R² and R^{2a} are independently hydrogen, optionally substituted alkyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹.

12. The compound of claim 11 wherein R^{a} is hydrogen or optionally substituted alkyl.

13. The compound of claim 12 wherein R^{2a} is hydrogen, optionally substituted alkyl, optionally substituted alkoxyalkyl, optionally substituted cycloalkyl, optionally substituted cyclohexenyl, optionally substituted bridged cycloalkyl, or tetrahydrofuranyl.

14. The compound of claim 13 wherein X is N.

15. The compound of claim 14 wherein the compound is
1-(3-(4-(hexyloxy)benzylamino)propyl)pyrrolidin-2-one;
(S)-2-(4-(hexyloxy)benzylamino)-3-methylbutan-1-ol;
(R)-2-(4-(hexyloxy)benzylamino)-3-methylbutan-1-ol;
(S)-1-(4-(hexyloxy)benzylamino)propan-2-ol;
(R)-2-(4-(hexyloxy)benzylamino)-3-methylbutan-1-ol;
(2R,3S)-3-(4-(hexyloxy)benzylamino)bicyclo[2.2.1]hept-5-ene-2-carboxylic acid;
(2S,3R)-3-(4-(hexyloxy)benzylamino)bicyclo[2.2.1]heptane-2-carboxylic acid;
(1R,6S)-6-(4-(hexyloxy)benzylamino)cyclohex-3-enecarboxylic acid;
(R)—N-(4-(hexyloxy)benzyl)-1-methoxypropan-2-amine;
3-((4-(hexyloxy)benzyl)(isopropyl)amino)propanoic acid;
(S)—N-(4-(hexyloxy)benzyl)tetrahydrofuran-3-amine;
N-(4-(hexyloxy)benzyl)-1-methoxybutan-2-amine;
2-(4-(hexyloxy)benzylamino)cycloheptanecarboxylic acid;
1-(4-(hexyloxy)benzylamino)-2-methylpropan-2-ol;
2-(4-(hexyloxy)benzylamino)cyclopentanecarboxylic acid;
(S)-2-(2-fluoro-4-(3-(trifluoromethyl)benzyloxy)benzylamino)-3-methylbutan-1-ol;
(R)—N-(2-fluoro-4-(3-(trifluoromethyl)benzyloxy)benzyl)tetrahydrofuran-3-amine;
(S)—N-(2-fluoro-4-(3-(trifluoromethyl)benzyloxy)benzyl)tetrahydrofuran-3-amine;
1-(2-fluoro-4-(3-(trifluoromethyl)benzyloxy)benzylamino)-2-methylpropan-2-ol;
(1-(2-fluoro-4-(3-(trifluoromethyl)benzyloxy)benzylamino)cyclopropyl)methanol;
1-(2-fluoro-4-(3-(trifluoromethyl)benzyloxy)benzylamino)cyclopropane carboxylic acid;
2-(2-fluoro-4-(3-(trifluoromethyl)benzyloxy)benzylamino)-2-methylpropanoic acid;
3-(2-fluoro-4-(3-(trifluoromethyl)benzyloxy)benzylamino)-2-methylpropanoic acid;
2-(2-fluoro-4-(3-(trifluoromethyl)benzyloxy)benzylamino)methyl)butan-1-ol;
N-(2-fluoro-4-(3-(trifluoromethyl)benzyloxy)benzyl)-3-methoxy-2-methylpropan-1-amine;
(R)-2-(2-fluoro-4-(3-(trifluoromethyl)benzyloxy)benzylamino)-3-methylbutan-1-ol;
(S)-1-(2-fluoro-4-(3-(trifluoromethyl)benzyloxy)benzylamino)propan-2-ol;
(R)-3-(2-fluoro-4-(3-(trifluoromethyl)benzyloxy)benzylamino)propane-1,2-diol;
(S)-3-(2-fluoro-4-(3-(trifluoromethyl)benzyloxy)benzylamino)propane-1,2-diol;
2-(2-fluoro-4-(3-(trifluoromethyl)benzyloxy)benzylamino)propane-1,3-diol;
2-(2-fluoro-4-(3-(trifluoromethyl)benzyloxy)benzylamino)-2-methylpropan-1-ol;
3-(2-fluoro-4-(3-(trifluoromethyl)benzyloxy)benzylamino)propane-1,2-diol;
(S)-1-(2-fluoro-4-(3-(trifluoromethyl)benzyloxy)benzylamino)propan-2-ol;
(S)-2-(2-fluoro-4-(3-(trifluoromethyl)benzyloxy)benzylamino)butan-1-ol;
1-(2-fluoro-4-(3-(trifluoromethyl)benzyloxy)benzyl)-4-methylpyrrolidine-3-carboxylic acid;
(R)-3-(4-((trimethylsilyl)ethynyl)benzylamino)propane-1,2-diol;
4-(4-((trimethylsilyl)ethynyl)benzylamino)butanoic acid;
(R)-2-(4-((trimethylsilyl)ethynyl)benzylamino)butanoic acid;
2-methyl-2-(4-((trimethylsilyl)ethynyl)benzylamino)propanoic acid;
2-methyl-3-(4-((trimethylsilyl)ethynyl)benzylamino)propanoic acid;
2-(4-((trimethylsilyl)ethynyl)benzylamino)propane-1,3-diol;
(S)-3-(4-((trimethylsilyl)ethynyl)benzylamino)propane-1,2-diol;
(R)-2-(4-((trimethylsilyl)ethynyl)benzylamino)propanoic acid;
(S)-2-hydroxy-3-(4-((trimethylsilyl)ethynyl)benzylamino)propanoic acid;
(S)-2-(4-((trimethylsilyl)ethynyl)benzylamino)butanoic acid;
2-(4-((trimethylsilyl)ethynyl)benzylamino)acetic acid;
3-(ethyl(4-((trimethylsilyl)ethynyl)benzyl)amino)propanoic acid;
(S)-2-(4-((trimethylsilyl)ethynyl)benzylamino)propanoic acid; or
(1R,3S)-3-(5-pentylpyrimidin-2-ylamino)cyclopentanecarboxylic acid.

16. The compound according to claim 2 wherein the compound is
2-(2-fluoro-4-(3-(trifluoromethyl)benzyloxy)benzyl)octahydro cyclopenta[c]pyrrole-3a-carboxylic acid.

17. The compound according to claim 1 wherein the compound is selective for the S1P₅ receptor and does not cause lymphopenia or immunosuppression at therapeutically relevant amounts of drug.

18. A method for treating or preventing conditions, disorders or deficits modulated by S1P₅ in treating or preventing a condition or disorder selected from a neurodegenerative disorder, attention deficit disorder, attention deficit hyperactivity disorder (ADHD), substance abuse including alcohol abuse, bipolar disorder, mild cognitive impairment, age-associated memory impairment (AAMI), senile dementia, AIDS dementia, Pick's Disease, dementia associated with Lewy bodies, dementia associated with Down's syndrome, schizophrenia, schizoaffective disorder, smoking cessation, diminished CNS function associated with traumatic brain injury, infertility, lack of circulation, need for new blood vessel growth associated with wound healing, ischemia, sepsis, neurodegeneration, neuropathic pain, inflammation and inflammatory disorders comprising administering a therapeutically effective amount of S1P₅ receptor ligand to the patient; and a method for use of treating or preventing a condition or disorder characterized by attention or cognitive dysfunction comprising administering a therapeutically effective amount of a S1P₅ ligands to a subject in need thereof in combination with a nicotinic acetylcholine receptor ligand or an acetylcholinesterase inhibitor comprising the step of administering to a subject in need thereof a therapeutically effective amount of one or more compounds of Formula (I), 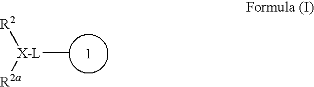
or a pharmaceutically acceptable salt, biologically active metabolite, solvate, hydrate, prodrug, enantiomer or stereoisomer thereof wherein
Ring 1 is optionally substituted aryl or optionally substituted heteroaryl;
L is —N(R^{a})—, —O— or C(R^{a})₂; wherein
R^{a} is independently H or optionally substituted alkyl;
X is N when L is C(R^{a})₂ or
X is CR^{a}; when L is —N— or —O—;
R² and R^{2a} are independently hydrogen, optionally substituted alkyl, optionally substituted alkenyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹; wherein
W is —O— or —S—; and
R¹¹ is —OR, —N(R)₂ or —SR; wherein
R is hydrogen, optionally substituted alkyl or haloalkyl; or
when X is N or C, R² and R^{2a} together with the carbon or nitrogen atom to which they are attached form an optionally substituted cycloalkyl, optionally substituted azetidine, optionally substituted pyrrolidine, optionally substituted piperidine or optionally substituted octahydrocyclopenta[c]pyrrolyl ring, provided that the azetidine ring formed by R² and R^{2a} together with the carbon or nitrogen atom to which they are attached is not substituted by
one or more phenyl;
phenyl and OH;
phenyl and —N(H)C(CH₃)₃;
—CH₂—O-optionally substituted pyridinyl;
—NH-optionally substituted quinazolinyl;
—O-optionally substituted pyridinyl;
—O—Si(CH₃)₂—C(CH₃)₃;
—C(OH)(4-(trifluoromethoxy)phenyl)(4-methoxyphenyl);
—C(OH)(4-(trifluoromethoxy)phenyl)(4-methoxyphenyl) and oxo;
—NH-isoquinolinyl;
optionally substituted alkyl and optionally substituted dioxolanyl;
oxo and —O-alkenyl;
oxo, two F and optionally substituted phenyl;
optionally substituted alkenyl and —O—C(O)-optionally substituted phenyl;
provided that when Ring 1 is optionally substituted phenyl, L is CH₂ X is N or C, and R² and R^{2a} together with the carbon or nitrogen atom to which they are attached form an optionally substituted cycloalkyl, or optionally substituted azetidine, Ring 1 is not substituted by
—CH═N—OCH₂CH₃;
—Cl and —NH₂;
—C(═O)CH₂CH₂-optionally substituted oxazolyl;
—NH—C(O)-alkenyl-optionally substituted pyridinyl;
—NO₂ and COOH—O-alkyl-optionally substituted oxazolyl;
O—CH₂-optionally substituted benzofuranyl;
—O—CH₂-optionally substituted phenyl;
—O—CH₂-optionally substituted pyrazolyl;
—O—CH₂-optionally substituted thienyl;
—O-optionally substituted (C₈)alkyl;
—O-optionally substituted (C₈)alkyl and halo;
—(C₆-C₁₂)alkyl wherein one or more carbons is optionally replaced by a nonperoxide oxygen;
—(C₆-C₁₂)alkenyl wherein one or more carbons is optionally replaced by a nonperoxide oxygen;
-pyrimidinyl substituted with oxo and —CF₂CF₃;
-optionally substituted 1,2,4 oxadiazole;
-optionally substituted thiazolo[5,4-b]pyridine;
-optionally substituted phenyl-CH₂—C(O)-optionally substituted pyrazolyl;
-optionally substituted phenyl-CH₂—C(O)-optionally substituted thiazolyl;
-optionally substituted phenyl-NH—C(O)-optionally substituted pyrazolyl;
-optionally substituted phenyl-NH—C(O)-optionally substituted tetrazolyl;
-optionally substituted phenyl-NH—C(O)-optionally substituted triazolyl;
-optionally substituted pyridinyl-CH₂—C(O)-optionally substituted pyrazolyl;
-optionally substituted pyridinyl-CH₂—C(O)-optionally substituted thiazolyl;
-optionally substituted pyridinyl-NH—C(O)-optionally substituted pyrazolyl;
-optionally substituted pyridinyl-NH—C(O)-optionally substituted tetrazoyl;
-optionally substituted pyridinyl-NH—C(O)-optionally substituted triazolyl;
-optionally substituted pyrimidinyl-CH₂—C(O)-optionally substituted pyrazolyl;
-optionally substituted pyrimidinyl-NH—C(O)-optionally substituted pyrazolyl;
-optionally substituted pyrimidinyl-NH—C(O)-optionally substituted triazolyl;
-optionally substituted phenyl-CH₂—C(O)-optionally substituted triazolyl;
provided that when Ring 1 is optionally substituted isoxazolyl or optionally substituted oxazolyl, Ring 1 is not substituted by
-optionally substituted phenyl-optionally substituted bicycle[2.2.1]heptanyl;
-optionally substituted phenyl-optionally substituted alkyl-optionally substituted phenyl;
provided that when Ring 1 is optionally substituted pyridinyl, Ring 1 is not substituted by
—C(O)—NH-optionally substituted phenyl;
—O-optionally substituted phenyl; and
provided that when Ring 1 is optionally substituted phenyl or naphthyl, L is CH₂ and NR² and NR^{2a} form an optionally substituted pyrrolidine ring, the pyrrolidine ring is not substituted by
—C(═O)(OH);
—F and —C(═O)(OH);
—OH and —C(═O)(OH);
—P(═O)(OH)(OH);
—OH and —P(═O)(OH)(OH);
—CH₂C(═O)(OH); or
tetrazolyl.

19. The method of claim 18, wherein said neuropathic pain is caused by peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain, pain associated with chronic alcoholism, hypothyroidism, uremia, multiple sclerosis, spinal cord injury, Parkinson's disease, epilepsy, vitamin deficiency, back pain, chronic low back pain, post-operative pain, injury-related pain, pain from spinal cord injury, eye pain, inflammatory pain, bone cancer pain, osteoarthritic pain, neuropathic pain, nociceptive pain, multiple sclerosis pain, post-stroke pain, diabetic neuropathic pain, neuropathic cancer pain, trigeminal neuralgia HIV-related neuropathic pain, phantom limb pain, fibromyalgia, or migraine.

20. The method of claim 18, wherein said neurodegenerative disorder is selected from the group consisting of neurodegenerative diseases selected from Alzheimer's disease, age-associated memory impairment, senile dementia, AIDS dementia, Pick's disease, dementia associated with Lewy bodies, dementia associated with Down's syndrome, Huntington's disease, Parkinson's disease, Amyotrophic Lateral Sclerosis, mild cognitive disorders, asphyxia, acute thromboembolic stroke, diminished CNS function associated with traumatic brain injury, focal and global ischemia, and transient cerebral ischemic attacks.

21. The method of claim 18, further comprising administering at least one additional therapeutic agent.

22. A method for inhibiting lysophosphatidic acid receptors 1, 2 or 3 comprising the step of administering to a subject in need thereof a therapeutically effective amount of one or more compounds of Formula (I), 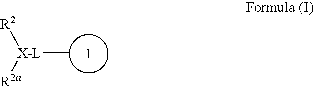
or a pharmaceutically acceptable salt, biologically active metabolite, solvate, hydrate, prodrug, enantiomer or stereoisomer thereof wherein
Ring 1 is optionally substituted aryl or optionally substituted heteroaryl;
L is —N(R^{a})—, —O— or C(R^{a})₂; wherein
R^{a} is independently H or optionally substituted alkyl;
X is N when L is C(R^{a})₂ or
X is CR^{a}; when L is —N— or —O—;
R² and R^{2a} are independently hydrogen, optionally substituted alkyl, optionally substituted alkenyl, optionally substituted alkoxyalkyl, optionally substituted cycloalkyl, optionally substituted cycloalkenyl, optionally substituted bridged cycloalkyl, optionally substituted heterocyclyl or —(CH₂)ₚC(═W)R¹¹; wherein
W is —O— or —S—; and
R¹¹ is —OR, —N(R)₂ or —SR; wherein
R is hydrogen, optionally substituted alkyl or haloalkyl; or
when X is N or C, R² and R^{2a} together with the carbon or nitrogen atom to which they are attached form an optionally substituted cycloalkyl, optionally substituted azetidine, optionally substituted pyrrolidine, optionally substituted piperidine or optionally substituted octahydrocyclopenta[c]pyrrolyl ring, provided that the azetidine ring formed by R² and R^{2a} together with the carbon or nitrogen atom to which they are attached is not substituted by
one or more phenyl;
phenyl and OH;
phenyl and —N(H)C(CH₃)₃;
—CH₂—O-optionally substituted pyridinyl;
—NH-optionally substituted quinazolinyl;
—O-optionally substituted pyridinyl;
—O—Si(CH₃)₂—C(CH₃)₃;
—C(OH)(4-(trifluoromethoxy)phenyl)(4-methoxyphenyl);
—C(OH)(4-(trifluoromethoxy)phenyl)(4-methoxyphenyl) and oxo;
—NH-isoquinolinyl;
optionally substituted alkyl and optionally substituted dioxolanyl;
oxo and —O-alkenyl;
oxo, two F and optionally substituted phenyl;
optionally substituted alkenyl and —O—C(O)-optionally substituted phenyl;
provided that when Ring 1 is optionally substituted phenyl, L is CH₂ X is N or C, and R² and R^{2a} together with the carbon or nitrogen atom to which they are attached form an optionally substituted cycloalkyl, or optionally substituted azetidine, Ring 1 is not substituted by
—CH═N—OCH₂CH₃;
—Cl and —NH₂;
—C(═O)CH₂CH₂-optionally substituted oxazolyl;
—NH—C(O)-alkenyl-optionally substituted pyridinyl;
—NO₂ and COOH—O-alkyl-optionally substituted oxazolyl;
—O—CH₂-optionally substituted benzofuranyl;
—O—CH₂-optionally substituted phenyl;
—O—CH₂-optionally substituted pyrazolyl;
—O—CH₂-optionally substituted thienyl;
—O-optionally substituted (C₈)alkyl;
—O-optionally substituted (C₈)alkyl and halo;
—(C₆-C₁₂)alkyl wherein one or more carbons is optionally replaced by a nonperoxide oxygen;
—(C₆-C₁₂)alkenyl wherein one or more carbons is optionally replaced by a nonperoxide oxygen;
-pyrimidinyl substituted with oxo and —CF₂CF₃;
-optionally substituted 1,2,4 oxadiazole;
-optionally substituted thiazolo[5,4-b]pyridine;
-optionally substituted phenyl-CH₂—C(O)-optionally substituted pyrazolyl;
-optionally substituted phenyl-CH₂—C(O)-optionally substituted thiazolyl;
-optionally substituted phenyl-NH—C(O)-optionally substituted pyrazolyl;
-optionally substituted phenyl-NH—C(O)-optionally substituted tetrazolyl;
-optionally substituted phenyl-NH—C(O)-optionally substituted triazolyl;
-optionally substituted pyridinyl-CH₂—C(O)-optionally substituted pyrazolyl;
-optionally substituted pyridinyl-CH₂—C(O)-optionally substituted thiazolyl;
-optionally substituted pyridinyl-NH—C(O)-optionally substituted pyrazolyl;
-optionally substituted pyridinyl-NH—C(O)-optionally substituted tetrazoyl;
-optionally substituted pyridinyl-NH—C(O)-optionally substituted triazolyl;
-optionally substituted pyrimidinyl-CH₂—C(O)-optionally substituted pyrazolyl;
-optionally substituted pyrimidinyl-NH—C(O)-optionally substituted pyrazolyl;
-optionally substituted pyrimidinyl-NH—C(O)-optionally substituted triazolyl;
-optionally substituted phenyl-CH₂—C(O)-optionally substituted triazolyl;
provided that when Ring 1 is optionally substituted isoxazolyl or optionally substituted oxazolyl, Ring 1 is not substituted by
-optionally substituted phenyl-optionally substituted bicycle[2.2.1]heptanyl;
-optionally substituted phenyl-optionally substituted alkyl-optionally substituted phenyl;
provided that when Ring 1 is optionally substituted pyridinyl, Ring 1 is not substituted by
—C(O)—NH-optionally substituted phenyl;
—O-optionally substituted phenyl; and
provided that when Ring 1 is optionally substituted phenyl or naphthyl, L is CH₂ and NR² and NR^{2a} form an optionally substituted pyrrolidine ring, the pyrrolidine ring is not substituted by
—C(═O)(OH);
—F and —C(═O)(OH);
—OH and —C(═O)(OH);
—P(═O)(OH)(OH);
—OH and —P(═O)(OH)(OH);
—CH₂C(═O)(OH); or
tetrazolyl.
